(19) United States
(12) Patent Application Publication
(10) Pub. No.: US 2015/0079045 A1
(43) Pub. Date: Mar. 19, 2015

(54) NPRCPS, PFDNCS AND USES THEREOF

(71) Applicant: Wuyi Kong, Sunnyvale, CA (US)

(72) Inventor: Wuyi Kong, Sunnyvale, CA (US)

(21) Appl. No.: 14/378,627

(22) PCT Filed: Feb. 12, 2013
(86) PCT No.: PCT/US2013/025767

§ 371 Date: Aug. 13, 2014

Related U.S. Application Data
(60) Provisional application No. 61/598,069, filed on Feb. 13, 2012.

(51) Int. Cl. A61K 35/12

(52) U.S. Cl. CPC A61K 35/12
USPC 424/93.7; 435/325

(57) ABSTRACT

Identification of a group of novel particle termed “non-platelet RNA-containing particles (NPRCP)” provides novel compositions, downstream products and therapeutic tools. In addition a group of mixed NPRCPs were identified that contain RNAs and proteins. NPRCPs do not have a nucleus and their membrane is not the typical eukaryotic cell membrane. Methods for isolation and enrichment are also provided.

### CROSS-REFERENCE TO RELATED APPLICATIONS

This application claims priority to International PCT Application PCT/US13/25767, filed on Feb. 12, 2013, which claims priority to U.S. Provisional Application No. 61/598,069, filed Feb. 13, 2012, the entire contents of which are incorporated herein by reference.

### FIELD OF INVENTION

Embodiments of the invention are directed to compositions of highly regenerative mammalian particles and their assembled products.

### BACKGROUND

A central challenge for research in regenerative medicine is to develop cell compositions that can help reconstitute damaged tissues and organs. Methods for regenerating or repairing damaged tissues may be used to address a variety of diseases, disorders, and injuries characterized by a loss or a deficiency in a particular cell type or a tissue that have more cell types. Such cell loss may be associated with trauma, ischemic injury, metabolic disease, or a degenerative disorder. Organ transplantation has conventionally been used to replace damaged or diseased tissues. Unfortunately, the supply of donor organs is limited. Even when donor organs are, available, rejection of the transplanted biological material can occur. Stem cells used for cell therapy also induce rejection, and therefore, blood from both donor and recipient has to be tested for tissue matching.

### SUMMARY

This Summary is provided to present a summary of the invention to briefly indicate the nature and substance of the invention. It is submitted with the understanding that it will not be used to interpret or limit the scope or meaning of the claims.

Embodiments of the invention are directed, inter alia, to the isolation, culturing and use of mammalian non-platelet RNA-containing particles (NPRCPs) and particle-fusion-derived non-nucleated cells (PFDNCs) assembled by NPRCPs (ATCC Patent Deposit Designation: PTA-13396).

NPRCPs comprise small RNAs and proteins and have a diameter of about 0.1 μm to about 10 μm, and surrounded by a thin membrane. NPRCPs are small vesicles containing proteins, small RNAs and microRNAs, and grow from less than about 1 μm up to about 7 μm. The size increase is possibly by taking the nutrients and particles from outside of the thin membrane.

In preferred embodiments, at least 5 types of different NPRCPs are identified (FIG. 2-6). They were listed as 1) Containing large core-like granulose type particles (FIG. 2). These type particles contain large nuclear granules (arrows). These nuclear granules have dense core-like structure and vary in sizes (0.1-0.5 μm). This type NPRCPs' sizes arrange from 2 μm to 5 μm. 2) The loose membrane type. These types of particles have extremely thin membrane outside and contain scattered dense nuclear granules inside the membrane. They also have a layered structure inside. Due to the flexibility of membrane structures, their shape can be very different. 3) The solid particle type. These types of particles do not have loose membrane. They contain small granules, dense nuclear materials and less dense areas. They are round shapes. Most nuclear granules locate at one side of the particles (see the 4-5 μm) or beneath the membrane. 4) The condensed material type. These types of particles contain dense materials that distribute evenly in the components. Some nuclear granules can be seen beneath the membrane. In addition, their membrane is thick and has irregular protrusions. 5) The uniform and not condensed type. These types of particles contain small nuclear granules and fiber-like structure that distribute evenly inside the components. These particles are in round shape with thin membranes.

In preferred embodiments, NPRCPs express surface markers comprising CD29 (integrin β1), CXCR4, c-kit, CD45, CD34, actin or combinations thereof (FIGS. 8A-8D to 10). They also contain DDX4/VASA, Oct4, sox-2 and tubulin in their components (FIG. 8A-8D), except small RNA and microRNA (FIG. 7).

In another preferred embodiment, less than 5% of isolated NPRCPs express E-cadherin on the surface.

In embodiments, NPRCP are released from larger sized Oct4-expressing, non-nucleated pre-cells (FIG. 13, 15, 16), however, these pre-cells can differentiate to nucleated cells, which is depended on their differentiation stages (FIG. 15 left, 17). At least two different NPRCP-releasing pre-stem cells were identified (FIG. 13). These pre-stem cells do not have distinct cytoplasmic components, and do not have distinct nucleus, either. They can release hundreds of small sized NPRCPs that further grow bigger in the extracellular environments by collecting circulating microRNAs, small RNAs, and proteins. These NPRCP-releasing pre-stem cells, in cultural condition, are characterized by a small cellular structure surrounded by numerous NPRCPs that are indifferent sizes (FIG. 17). However, in the blood, the NPRCP-releasing pre-cells are characterized as either spindle shape or round shape cellular structures that do not have nucleus (arrows in FIG. 13).

In embodiments, NPRCPs comprise at least three different shapes: spindle shape, round shape and short-rod shapes (FIG. 14).

In other embodiments, NPRCPs fuse together and become a cellular structure (FIGS. 19-20) having sizes from about 7 μm to about 15 μm, depending on the numbers of fused NPRCPs (FIGS. 31A, 31B). These cellular structures have never been described elsewhere and therefore, have been termed “particle-fusion-derived non-nucleated cells” (PFDNCs). PFDNCs exhibit amoebic-like motion (FIGS. 21A-21F). PFDNCs express Oct4, SOX2, integrin β1, tubulin, actin and DDX4 (FIG. 22). PFDNCs can twist their bodies and use the loose thin membrane to capture large or small particles to increase their body size. In addition, PFDNCs squeeze themselves to get inside eukaryotic cells to collect the genetic material for their lineage development (FIG. 27).

In other embodiments, PFDNCs do not have a cytoplasmic membrane and nucleus. PFDNCs are derived from collection of cytoplasmic material by NPRCPs. Electron microscopy images of 2 pre-cellular structures, each about 5 μm (A) and 8 μm (B) with an NPRCP in the center (arrows), which evidences that the core of PFDNCs are derived from NPRCPs (FIGS. 23A, 23B).

In other embodiments, PFDNCs can be released by some large cellular structures that are fused from multiple NPRCPs, and expressing Oct4, sox2 and tubulin (FIG. 32, 33A-33C).

In embodiments, after collecting materials from the eukaryotic cells (FIGS. 23A, 23B), the size of PFDNCs increase to a diameter of about 10 μm to about 20 μm and become more differentiated PFDNCs (FIG. 21G-21I). These more differentiated PFDNCs then fuse to each other and acquire a cytoplasmic membrane, which can be derived from damaged cells (FIG. 28), fusion of circulating membrane vesicles or other differentiated PFDNCs, they transform into mesenchymal-like pre-cells (FIG. 28-30). The genetic material of the cell that the PFDNC squeezed into and collected from determines the lineage type of PFDNC transformed cells (FIGS. 23A, 23B). The PFDNC-derived new cells are stem cells that transiently express Oct4 (FIG. 26).

Without wishing to be bound by theory, the cellular formation of NPRCPs and PFDNCs is via at least three ways. One is direct transdifferentiation that is by PFDNCs transforming into nucleated cells (FIGS. 25-26), represented by interstitial cells, endothelial cells, and others. Another way is by fusion-differentiation by multiple, or by hundreds of NPRCPs fusing into large patch-like structures (FIGS. 31A, 31B, 33A-33C) that undergo nuclear programming and become a tissues, represented by renal ducts, pancreatic g cells and acina ducts, intestine crypts, neurons, smooth muscle fibers, cardiac muscle fibers, and others. The third way is via NPRCP or PFDNC fusion with nucleated cells and release oct4, sox2, DDX4 and other proteins and RNAs to induce these nucleated cells to become stem cells or healthy cells (FIG. 92).

In other embodiments, in acute ischemic conditions, such as during large tissue damage, multiple NPRCPs can also fuse into large patch-like structures that further undergo nuclear programming and differentiate into large structures having multiple cells, such as renal ducts (FIGS. 42A-42J, 43A-43B, 44A-44F), neurons (FIGS. 57-60), cardiac myoblasts (FIGS. 77-79), hair follicles (FIG. 69-70) and others.

In toxic chemical-induced cellular damages, i.e., cellular damages are not restricted in certain areas, scattered NPRCPs can migrate to the damaged cells and fuse with these cells. NPRCPs change their shape into a short-rode with a long tail (FIG. 92) that can release oct4, sox2, DDX4 and other unidentified proteins. Released oct4, sox2 and DDX4 can induce toxic or stressed cells into healthy or stem cells by function on the downstream proteins to reprogramming these cells. Without wishing to be bound by theory, it is thought that the functions of NPRCP are by NPRCP's natural released RNA and proteins and not by gene transfection.

In embodiments, NPRCPs and PFDNCs regenerate ischemic damaged kidneys (FIGS. 39A-39G, 40-50). NPRCPs migrate to the ischemic damaged kidneys by circulation. NPRCPs can extravasate to the damaged areas and regenerate interstitial cells, glomeruli and renal ducts. NPRCPs can fuse and directly transdifferentiate into interstitial cells. They can form into new endothelial cells in the glomeruli. Multiple NPRCPs aggregate and fuse to become a larger mass that further differentiates into renal ducts with multiple cells.

In other embodiments, NPRCPs and PFDNCs regenerate ischemic damaged brain neurons (FIG. 51-65). NPRCPs migrate to the ischemic damaged brain by circulation (FIG. 51-52). In the brain, multiple NPRCPs extravasated and aggregate (FIG. 53-54). Aggregated NPRCPs start to protrude the dendrites on the 7 days after the transplantation. Meanwhile, dots-shaped nestin has linked together and become fiber-like structures (FIG. 55-59). Except forming dendrites, NPRCPs also aggregate to form axons. However, axon formation appears slower and also in different patterns. The aggregated NPRCPs surrounded by a layer of nucleated cells are identified 12 days after transplantation (FIG. 60-61). Further, aggregated NPRCPs fuse into cellular structures (FIG. 62). Axon formation appeared from 10 weeks after NPRCP transplantation. Images show GFP and nestin are co-expressed on an axon-like structure 10 weeks after NPRCP transplantation (FIG. 64-65). This data indicate that NPRCPs can regenerate axon, however, in a slower procedure.

In other embodiments, NPRCPs and PFDNCs regenerate wounded skin (FIGS. 66-72). NPRCPs form into hair bulbs have migrating to the wounds. Hair bulb-shaped, non-nucleated cells that are wrapped in hyaline layer are embedded in the plasma at the wound edge, indicating that these hair bulbs are extravasated (FIGS. 66-67). NPRCPs regenerate epithelial layer. NPRCPs appear at the epithelial layer, not only at the basal layer and also at the surface layer (FIG. 68). NPRCPs regenerate hair follicles (FIGS. 69-70) and skin connective tissues or dermis (FIGS. 71-72).

In other embodiments, NPRCPs and PFDNCs regenerate smooth muscle (FIGS. 73-74).

In other embodiments, NPRCPs and PFDNCs regenerate ischemic damaged hearts (FIGS. 75-80). Intravenous injected NPRCPs appear in the ischemic damaged hears in either aggregated forms or inside the blood vessels at 2 weeks after the transplantation (FIG. 75-76). Aggregated NPRCPs fuse into cardioblasts that also express smooth muscle actin, the stem cell marker. Smooth muscle actin stains as particle-shape, not fiber-like, indicating the fiber-shape is expressed on the differentiated cells. DAPI stain indicates that the newly fused NPRCPs are forming nuclei (arrows in FIG. 76). Aggregated NPRCPs form into cardiac myoblasts. By 4 weeks after the transplantation, no aggregated NPRCPs were detected, instead, GFP-expressing cardioblasts are found in 4-week hearts and 8 weeks hearts (FIG. 77-80).

In other embodiments, NPRCPs and PFDNCs regenerate intestine (FIGS. 81-85). NPRCPs were transplanted via tail-vein injection. Small intestine was collected 7 days after NPRCP transplantation. Under lower magnification, aggregated GFP-expressing NPRCPs were found only at the crypt areas (FIG. 81). Higher magnified images (FIG. 82) show that Oct4 does not colocalize to NPRCP (arrows in the upper image), while sox2 (arrows in the lower image) co-localizes to NPRCP only at the aggregation stage, but not at the fusion stage. Aggregated NPRCPs in the tiny dots-shapes enter the crypts through the spaces between the cells (FIG. 83). They remain in their aggregated shape after arriving at the crypts where they initiate their fusion and further differentiation (FIG. 84). NPRCP-derived cells have a high proliferating rate (FIG. 85).

In other embodiments, NPRCPs and PFDNCs regenerate toxic damaged liver (FIG. 86A-86D). Aggregated NPRCPs appear in the toxic chemical damaged liver 4 days after transplantation. NPRCPs regenerate hepatocytes by either aggregation-fusion pattern (FIG. 86 A-B) or by releasing regulatory factors to the cells as described (FIG. 86C-86D).

In other embodiments, NPRCPs and PFDNCs regenerate toxic damaged pancreases (FIGS. 87-91). GFP-expressing NPRCPs were found as grouped in the pancreatic tissues 2 days after transplantation. Insulin stained as fine particles in the GFP-expressing NPRCPs. A hollow structure can be seen in the center of aggregated NPRCPs, indicating that the nucleus of this cell has not formed yet (FIG. 87-88). Scattered GFP-expressing NPRCPs were found in the pancreas 2 days after transplantation. These NPRCPs locate closely with Oct4-expressing particles, evidencing that NPRCPs are regenerate pancreatic acina cells (FIG. 89). Because the expression GFP and insulin on the cells has a time gap, Oct4 expression was further studied on the pancreatic island cells. Again, Oct4 is expressed on the cells at the edge of the island, while the insulin-expressing cells are not co-localizing with the oct4-expressing cells. These data evidence that pancreatic B cells are not stem cells. These cells are fully differentiated and do not have the co-expression with the stem cell markers (FIG. 91).

Other aspects of the invention are described infra.

### BRIEF DESCRIPTION OF THE DRAWINGS

Section 1 (FIGS. 1A, 1B-12) describes the characteristics of NPRCPs, including their morphologies, observed by electron microscopy and standard light microscopy, their specific marker expression and morphology in cultural conditions. Section 2 (FIGS. 13-18) describe the origin of NPRCPs. NPRCPs are released by oct4-expressing pre-stem cells. Section 3 (FIGS. 19-24A-24C) describes PFDNCs formation and characterization, including their morphologies and surface markers. Section 4 (FIGS. 25-30) describes the single nucleate cell formation by PFDNC's direct transdifferentiation. Section 5 (FIGS. 31A, 31B-34) describes the multiple nucleated cell formation by PFDNC's fusion-differentiation. Section 6 (FIG. 35) describes the other types of blood NPRCPs that regenerate other blood type cells. Section 7 (FIGS. 36A, 36B) proves the NPRCP purification techniques in human umbilical cord blood. Section 8 (FIGS. 37A-37J, 38) shows that mouse NPRCPs can be purified and have similar expression as that in human. Section 9 (FIG. 39A-39G, 40-50) proves NPRCPs' in vivo regeneration in ischemic damaged kidneys. Section 10 (FIGS. 51-65) evidences the in vivo neuron regeneration by transplanted NPRCPs in ischemic brain damaged mice. Section 11 (FIGS. 66-72) evidences the in vivo skin and hair follicle regeneration by transplanted NPRCPs in mouse skin wounds. Section 12 (FIGS. 73-74) evidences the in vivo smooth muscle regeneration by transplanted NPRCPs in mouse skin wounds. Section 13 (FIGS. 75-80) evidences the in vivo cardiac cell regeneration by transplanted NPRCPs in ischemic damaged mouse hearts. Section 14 (FIGS. 81-85) evidences in vivo intestinal cell regeneration by transplanted NPRCPs. Section 15 (FIGS. 86A-86D) evidences the hepatocytes regeneration of transplanted NPRCPs in toxic chemicals damaged mouse liver. Section 16 (FIGS. 87-91) evidences the in vivo pancreatic regeneration by transplanted NPRCPs in mouse models. Section 17 (FIG. 92) evidences that under healthy condition, PFDNCs may regulate cell reprogramming by releasing the transcription factors or proteins, such as oct4 and sox2, the well-known factors for cellular reprogramming. PFDNCs can release regulatory factors by budding off them through their long-tail-like structures.

FIGS. 1A, 1B: NPRCPs contain both proteins and RNA. FIGS. 1A and 1B show that cultural enriched NPRCP stained with haematoxylin and eosin (H&E) and haematoxylin only. All NPRCPs are smaller than 5 μm. Hematoxylin stains for nuclear materials and eosin stains mostly for proteins. Small NPRCPs stain positive for H&E, with more haematoxylin stains at one side of the membrane (arrows) and eosin in the rest areas. Scale bars=5 μm.

FIGS. 2-6 show that NPRCPs are a mixed population. To identify the detailed structure of these NPRCPs, electron microscopy was used for examination. EM images revealed more than 5 types of different particles. They were listed as 1) Containing large core-like granulose type particles (FIG. 2). These type particles contain large nuclear granules (arrows). These nuclear granules have dense core-like structure and vary in sizes (0.1-0.5 μm). This type of NPRCPs range from about 2 μm to about 5 μm in size.

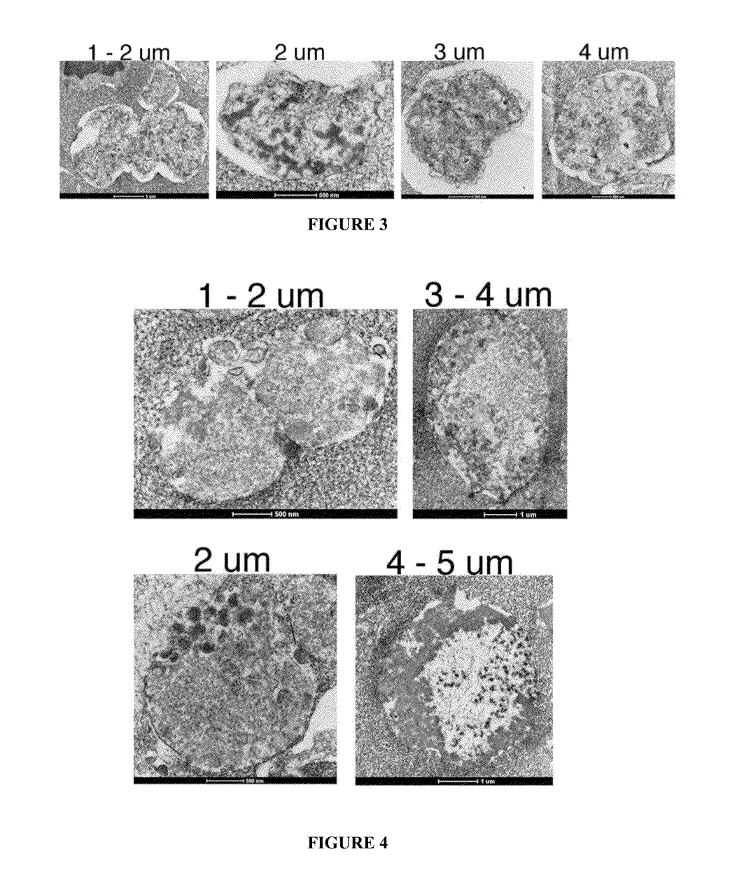

2) The loose membrane type (FIG. 3). This type of NPRCPs has an extremely thin outer membrane and contains scattered dense nuclear granules inside the membrane. They also have layered structures inside. Due to the flexibility of membrane structures, their shape can vary.

3) The solid particle type (FIG. 4). This type of NPRCPs do not have loose membranes. They contain small alveoli small granules, dense nuclear materials and less dense areas. They are mostly rounded in shape. Most nuclear granules locate at one side of the particles (see the 3-4 um and 4-5 μm panels) or beneath the membrane.

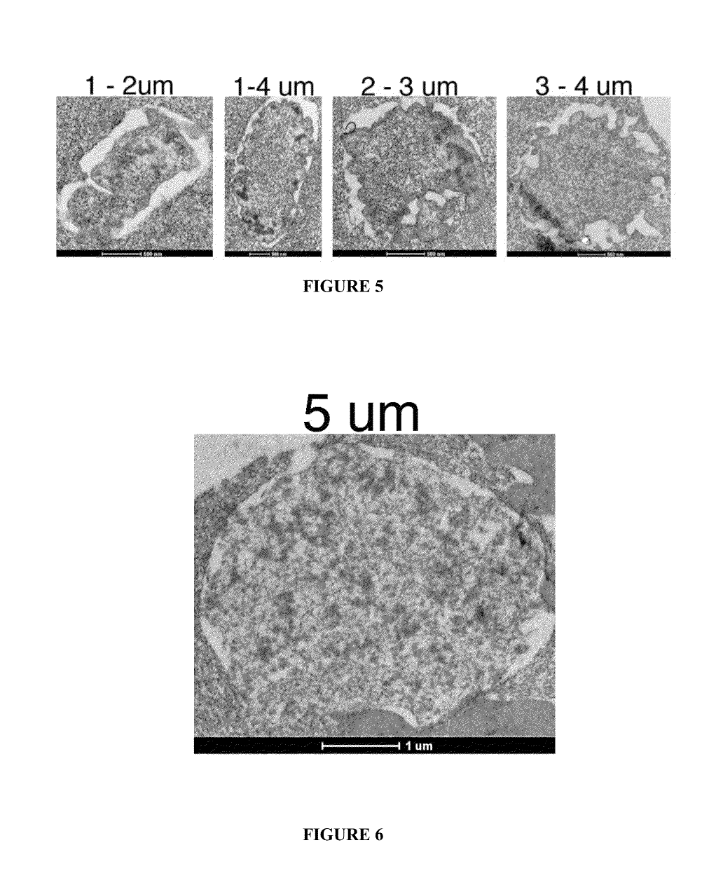

4) The condensed material type (FIG. 5). This type of NPRCPs contains dense materials that distribute evenly in the body of NPRCPs. Some larger dense nuclear granules can be seen close to the membrane. In addition, their membrane is thick and has irregular protrusions.

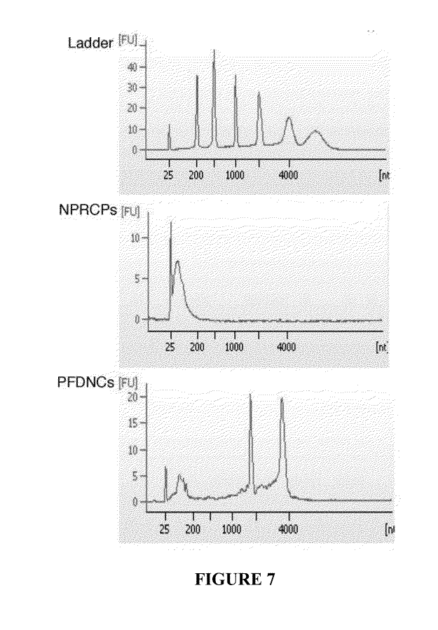

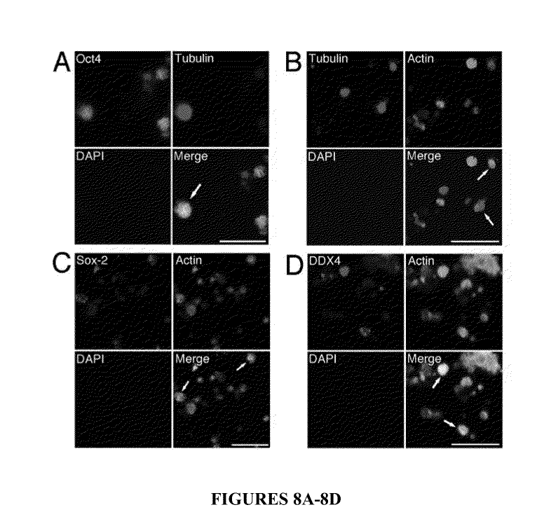

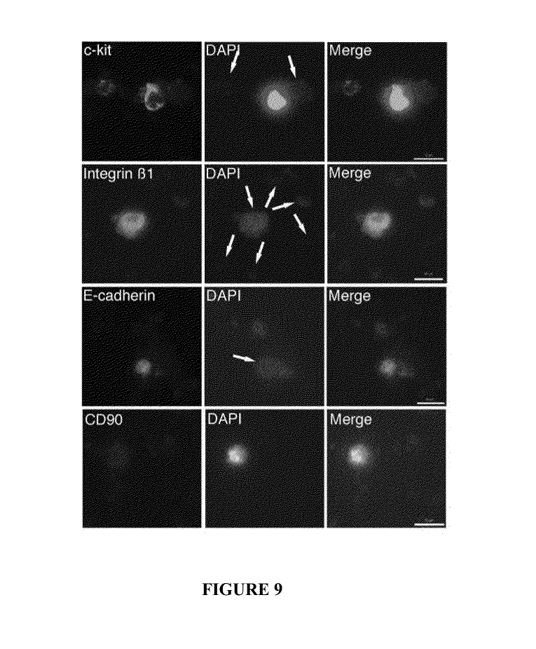

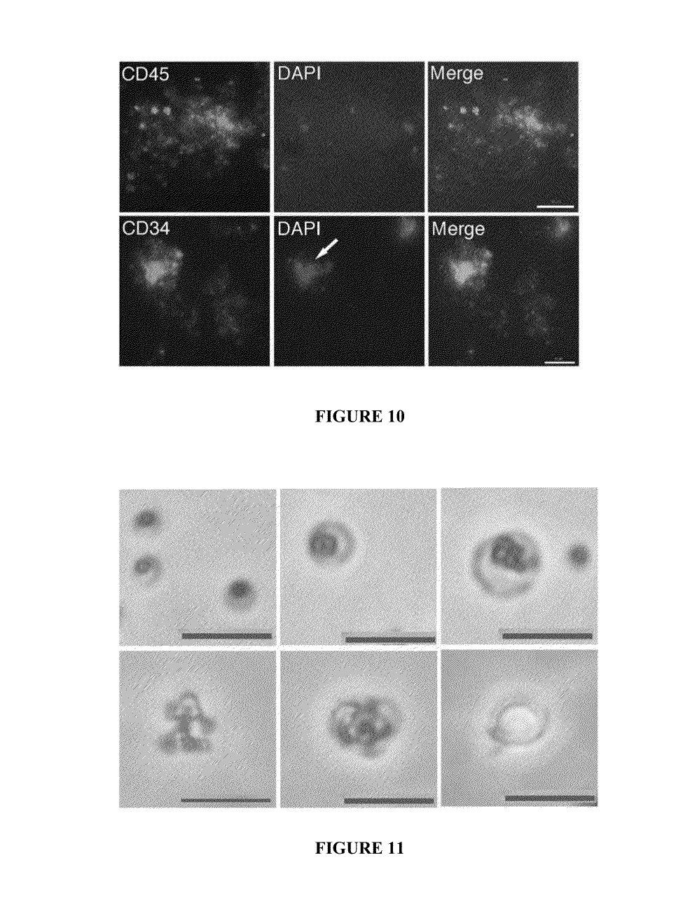

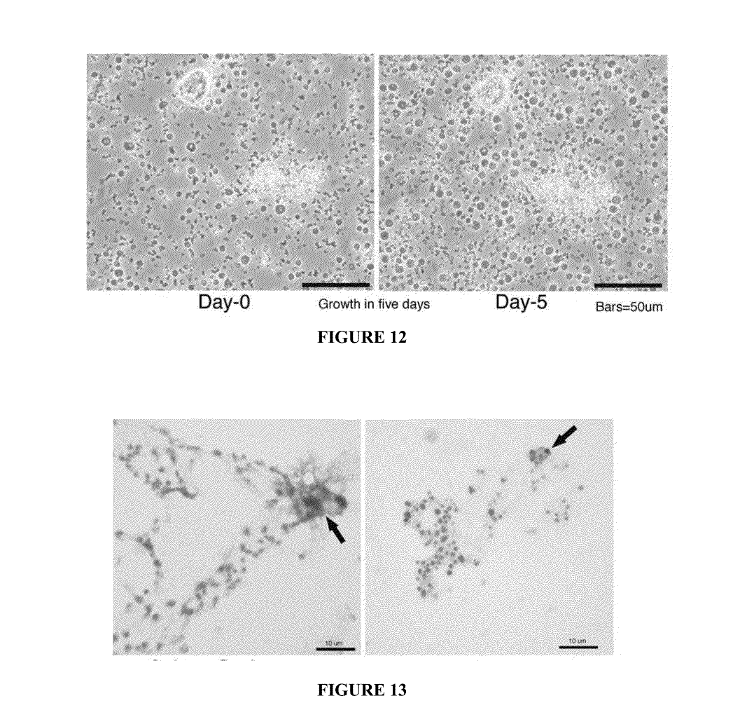

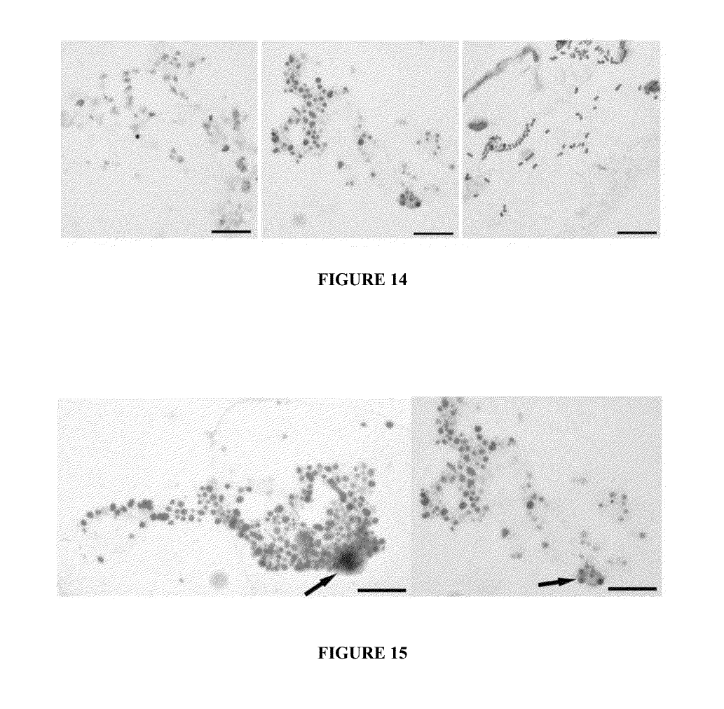

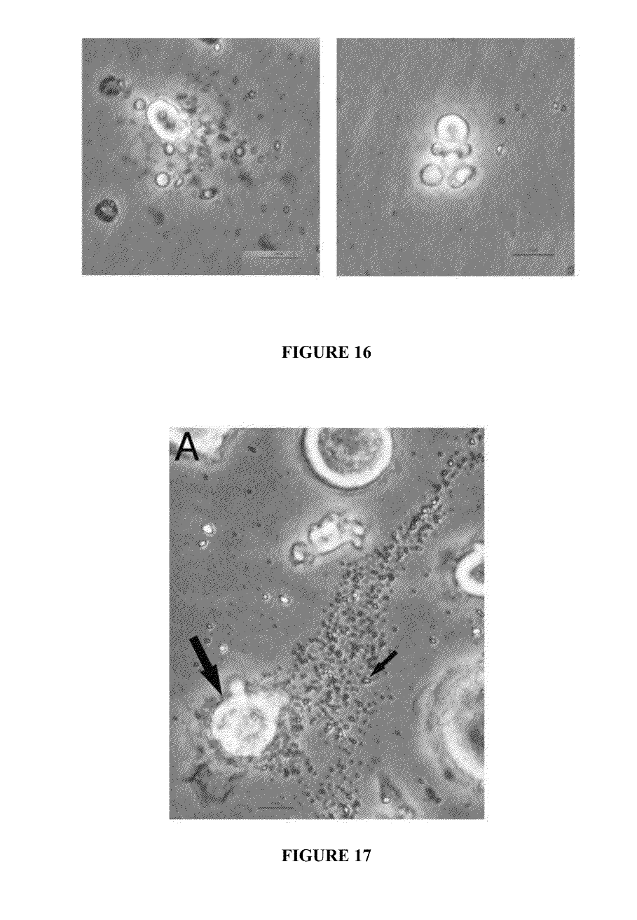

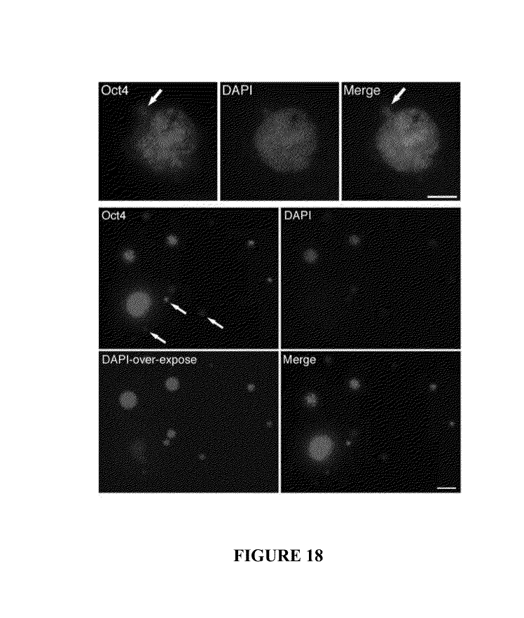

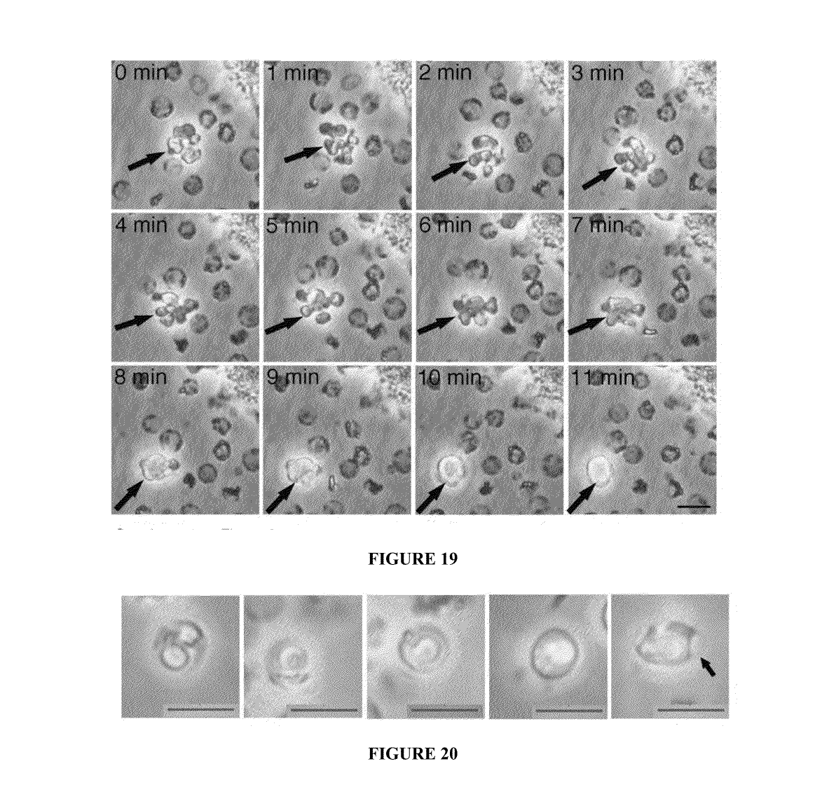

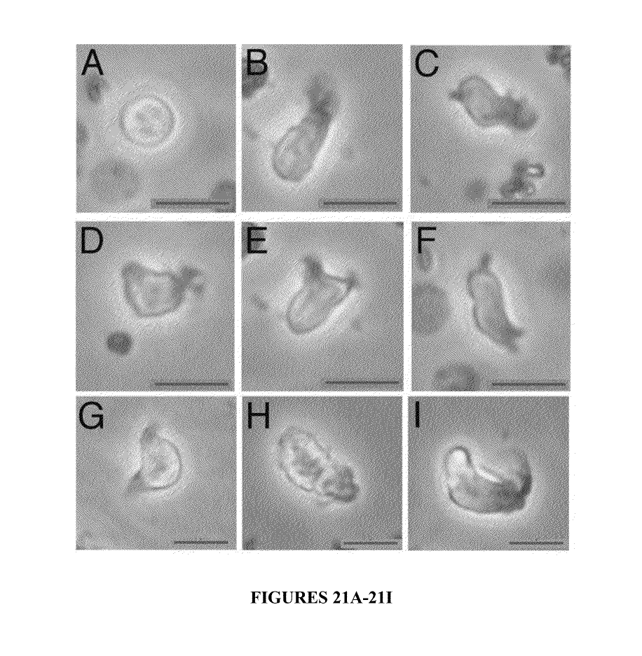

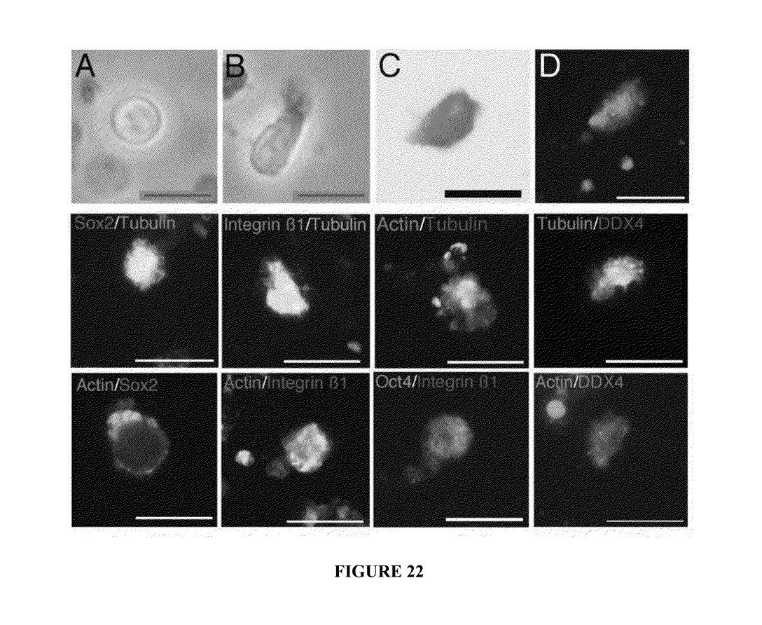

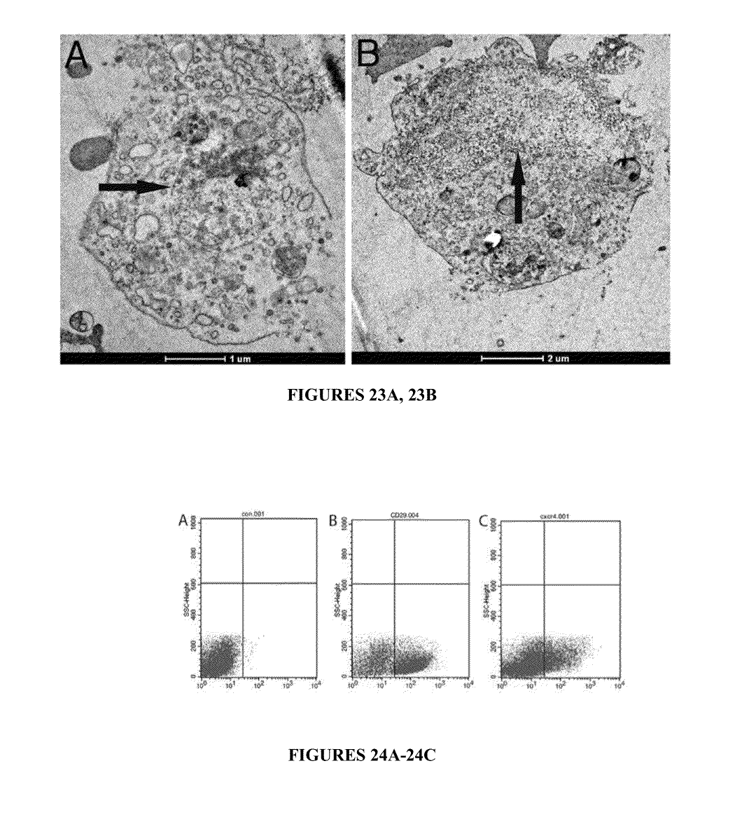

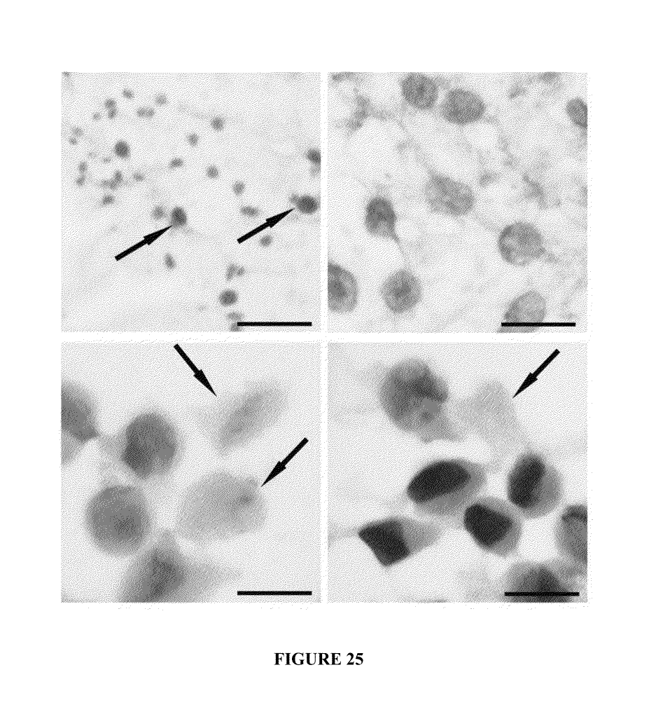

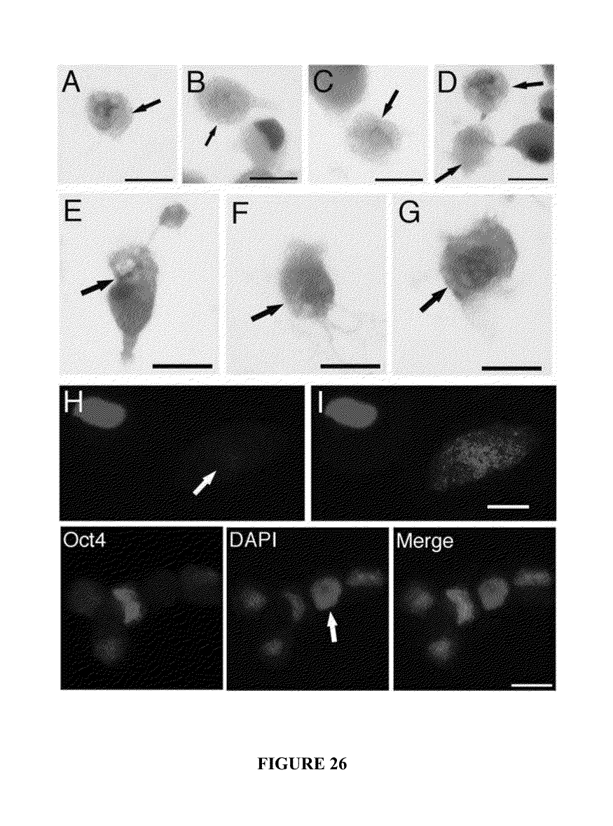

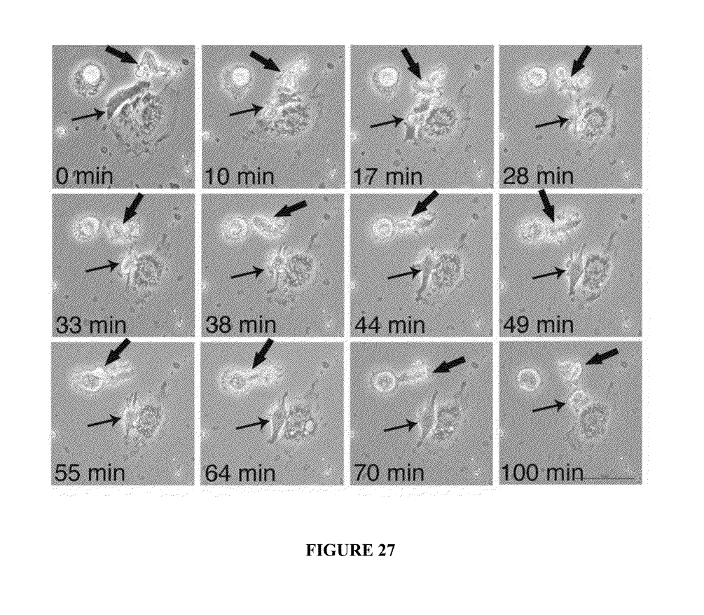

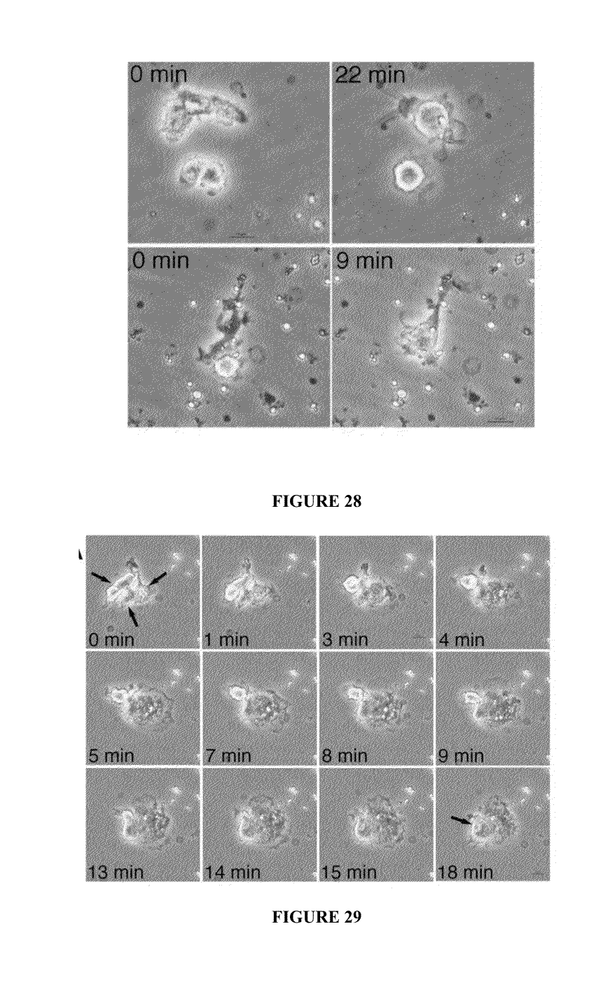

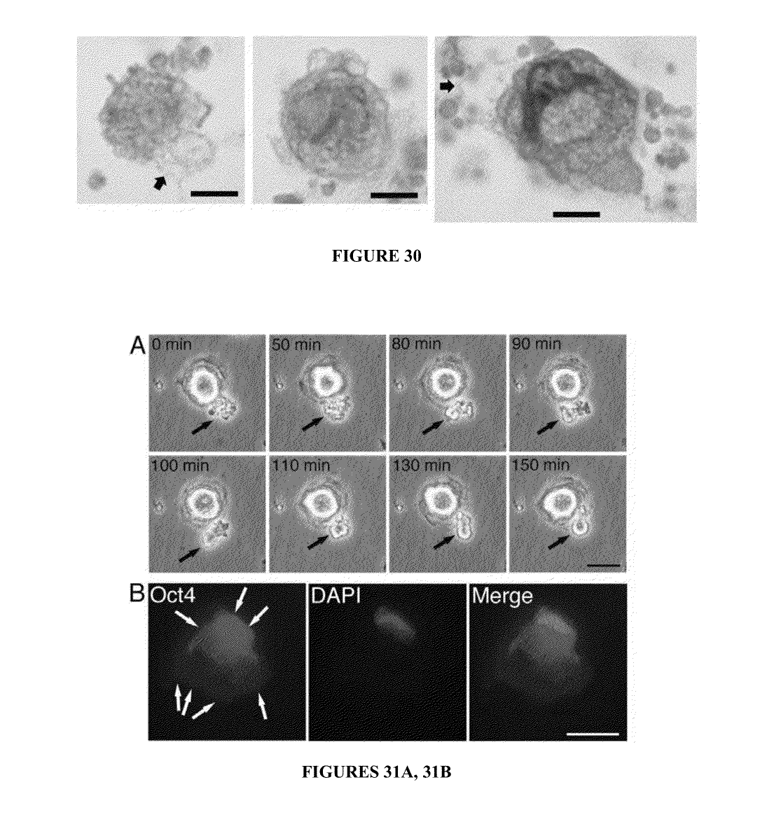

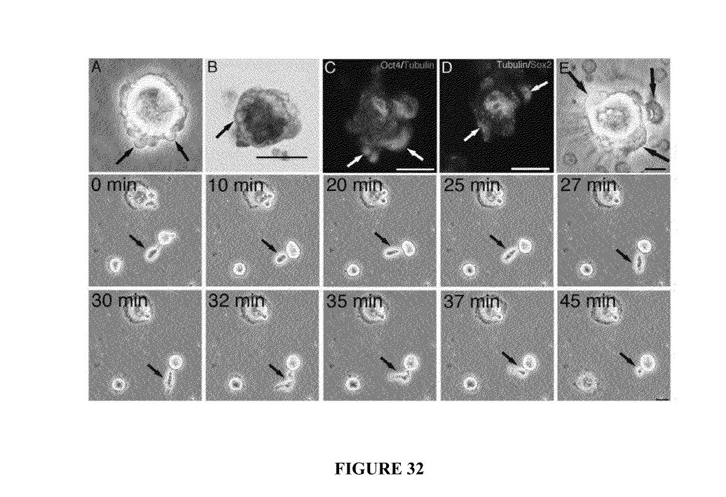

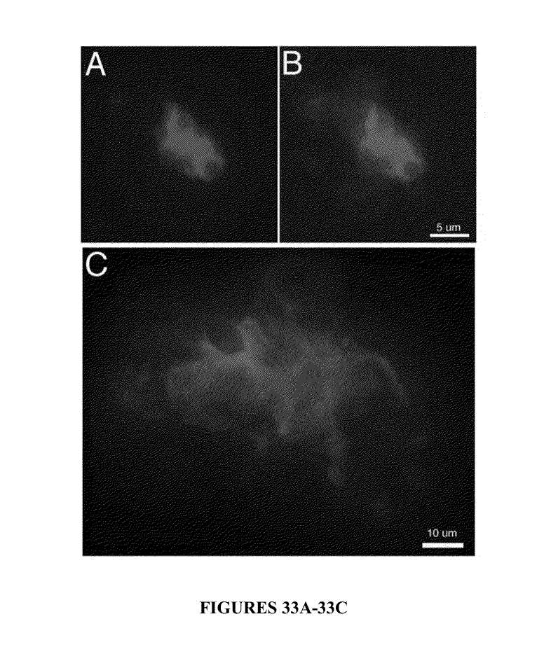

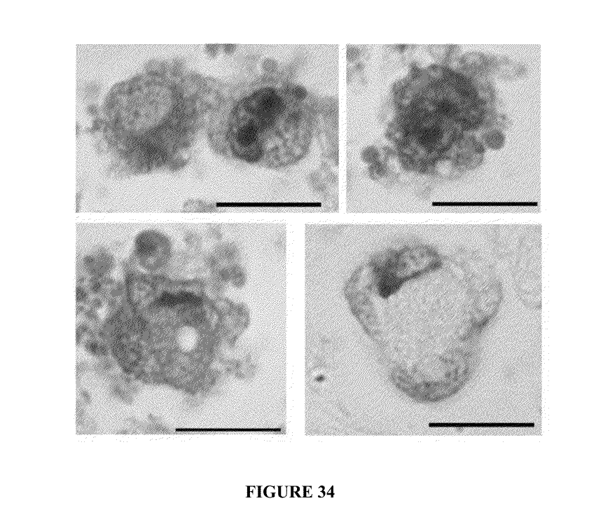

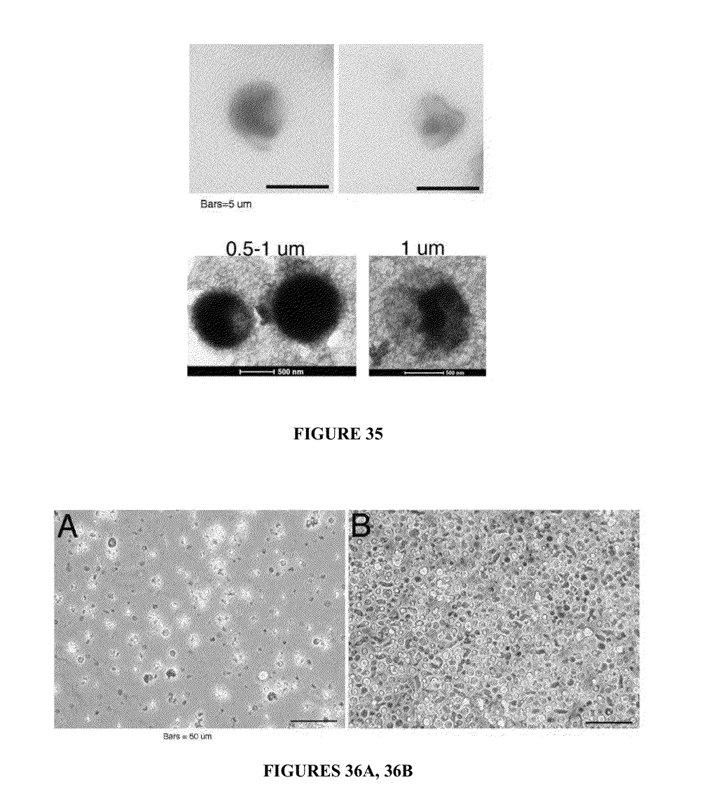

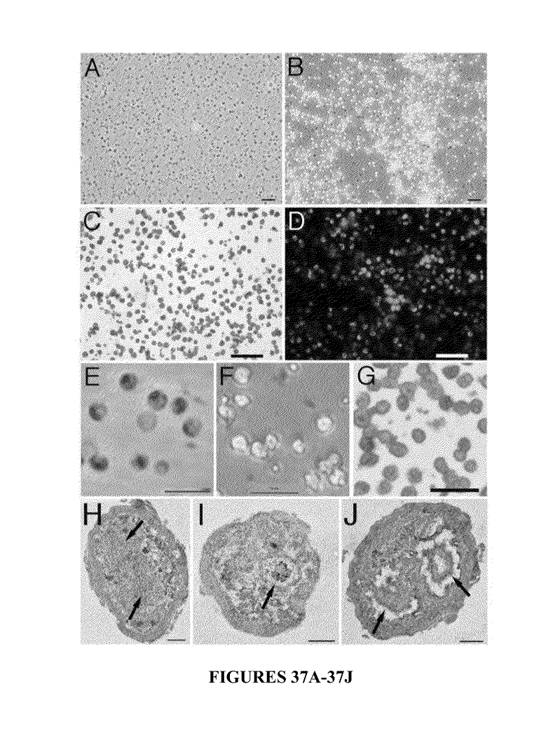

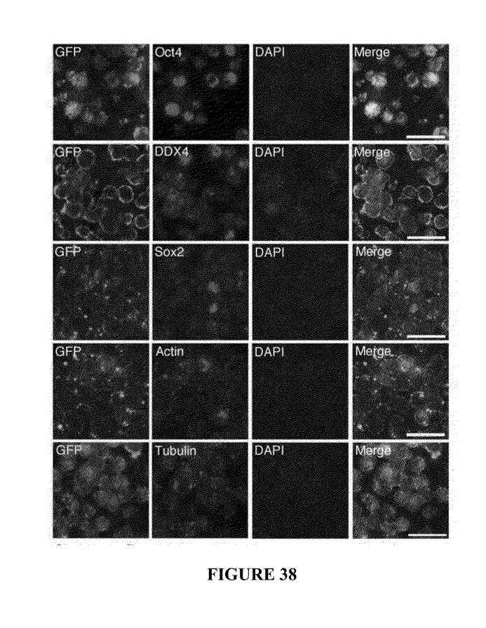

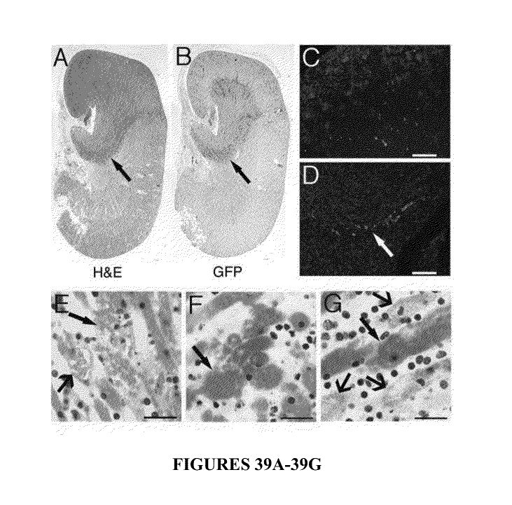

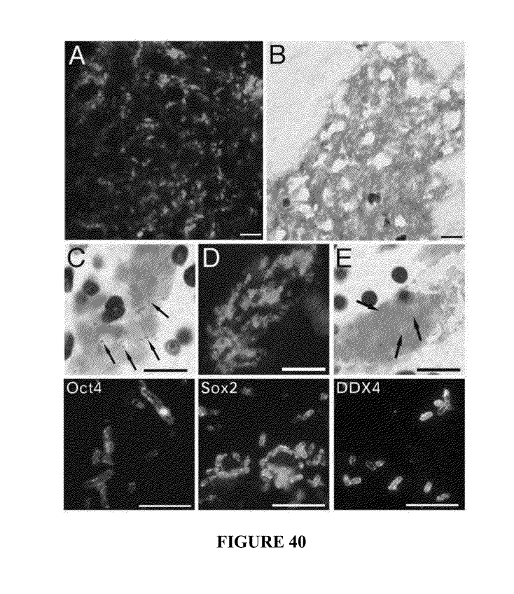

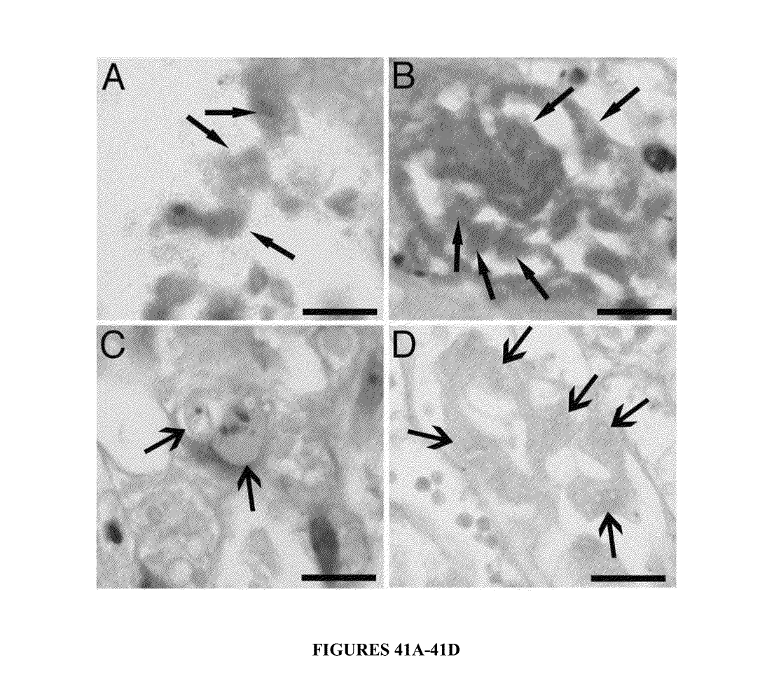

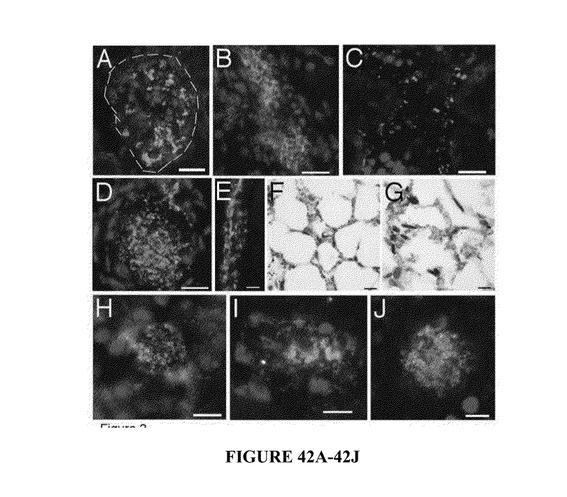

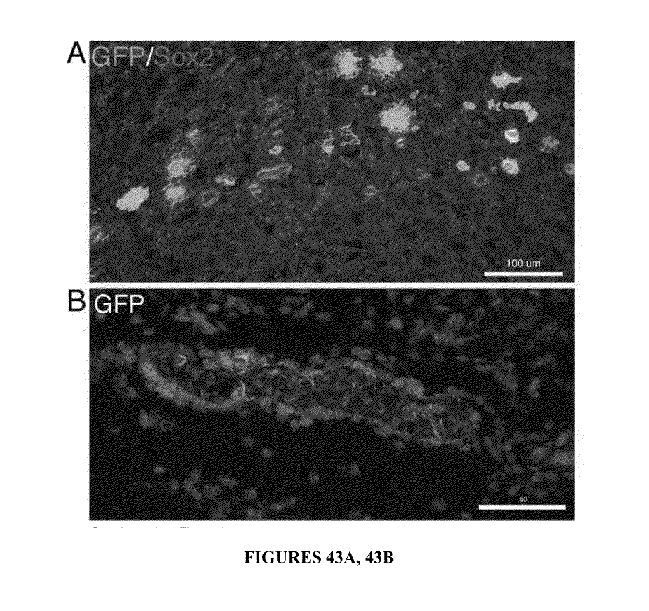

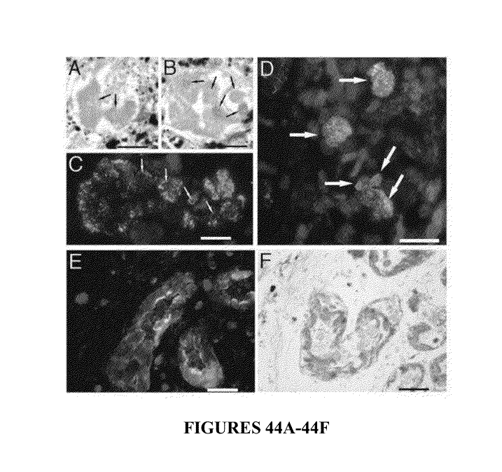

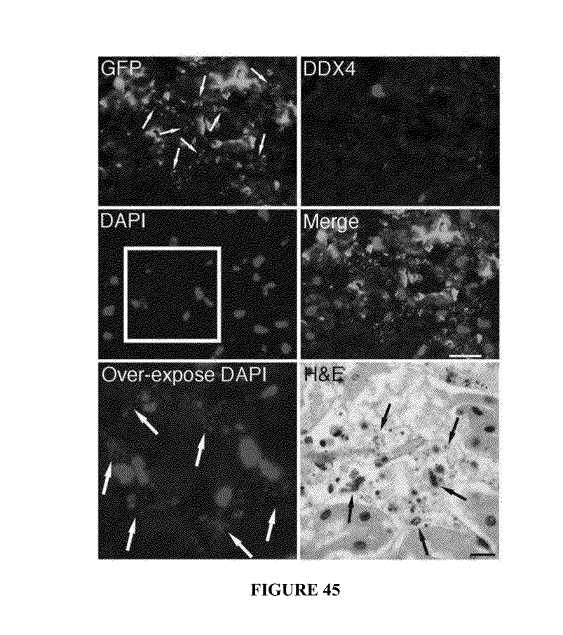

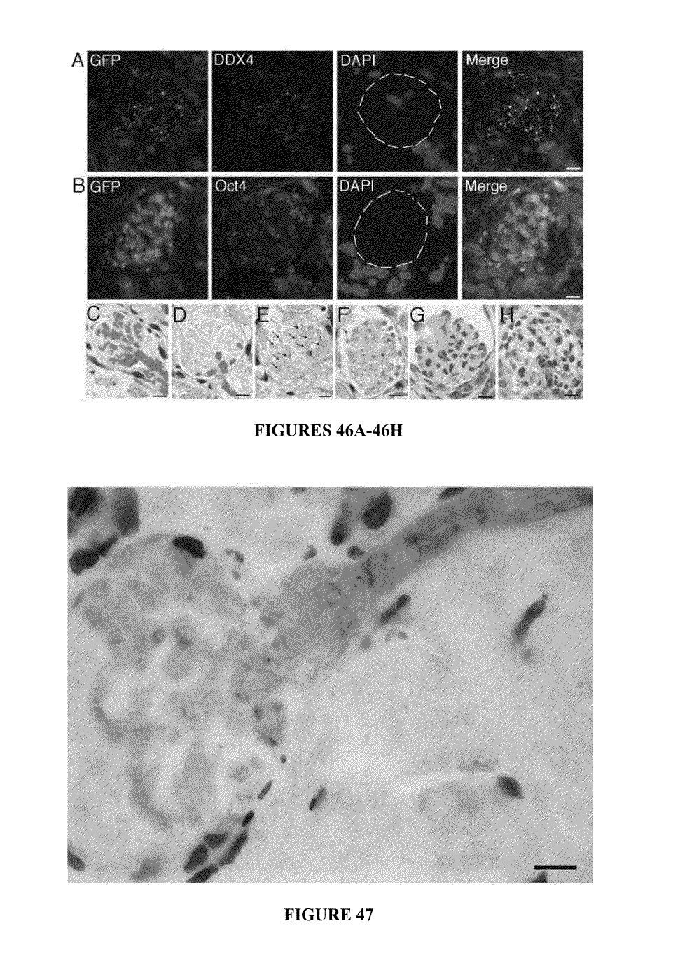

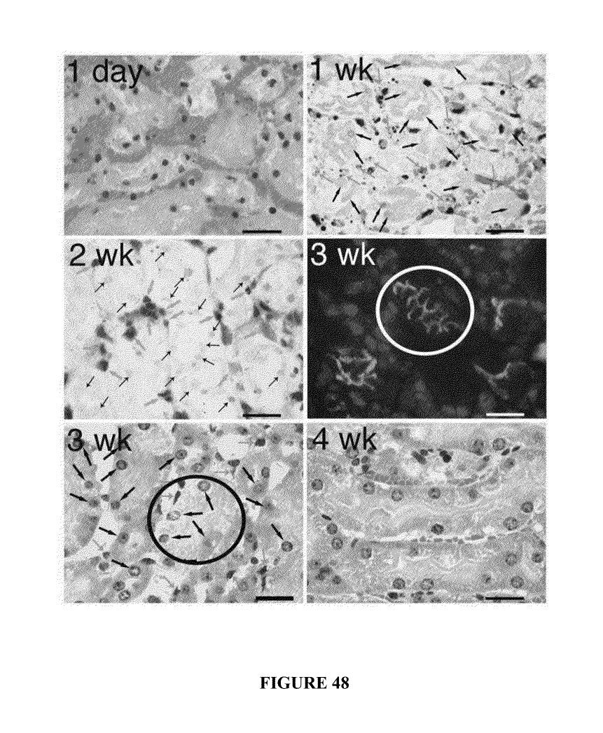

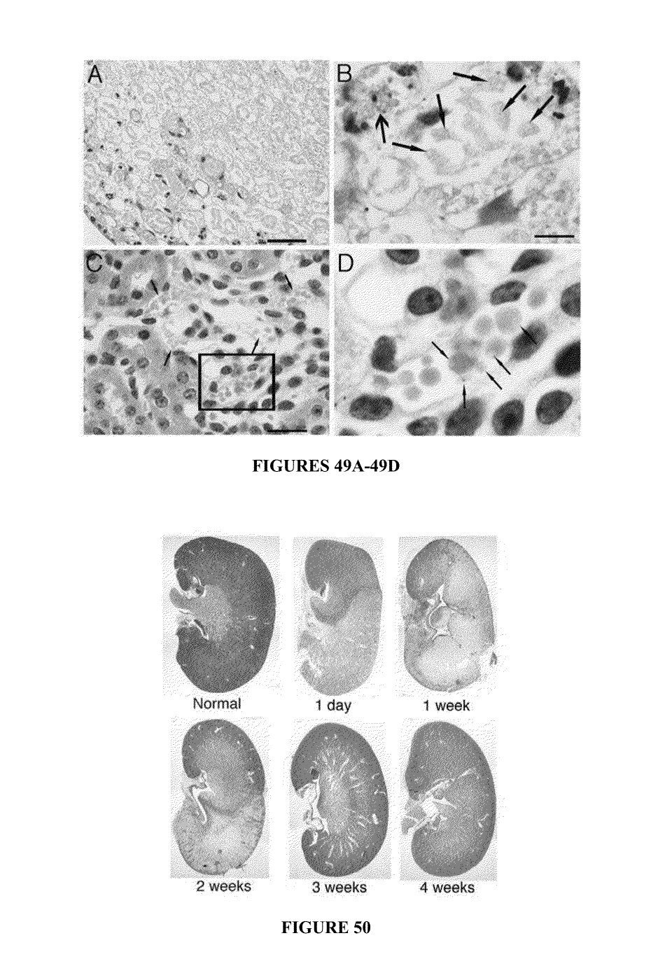

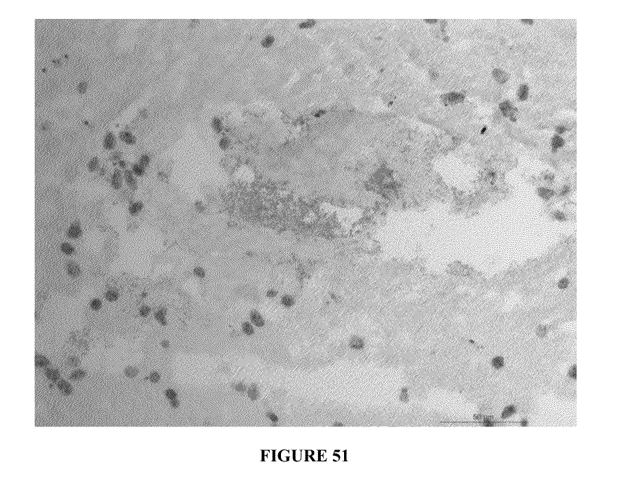

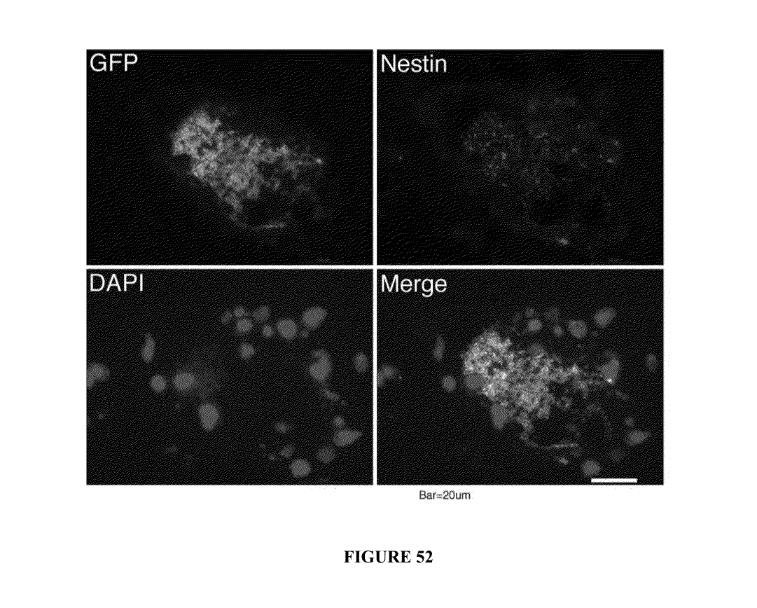

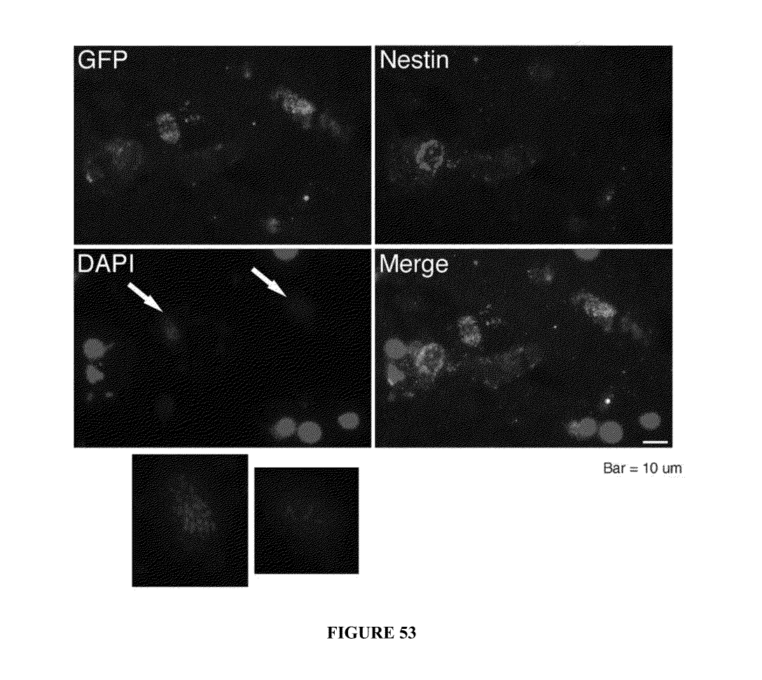

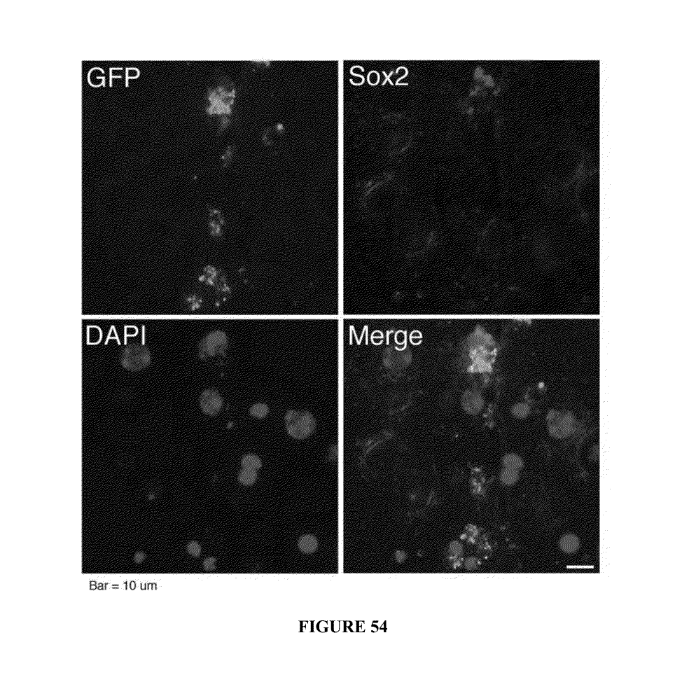

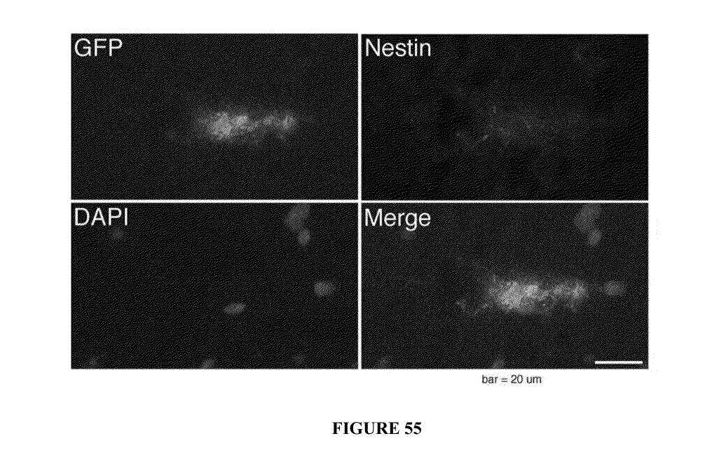

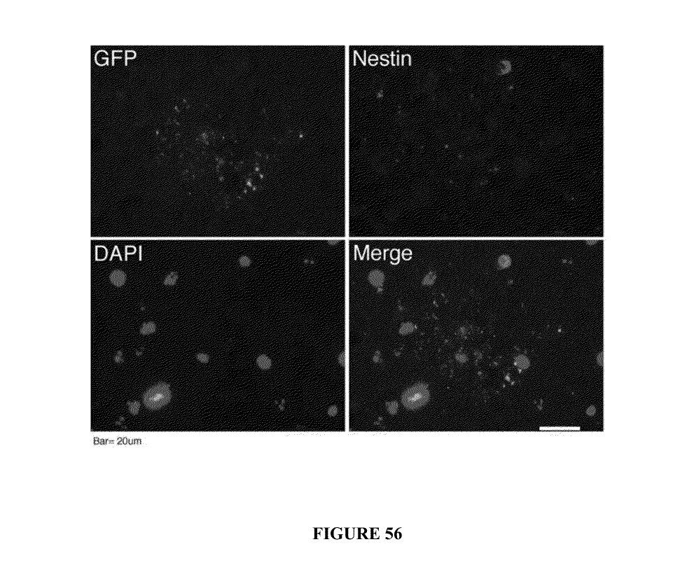

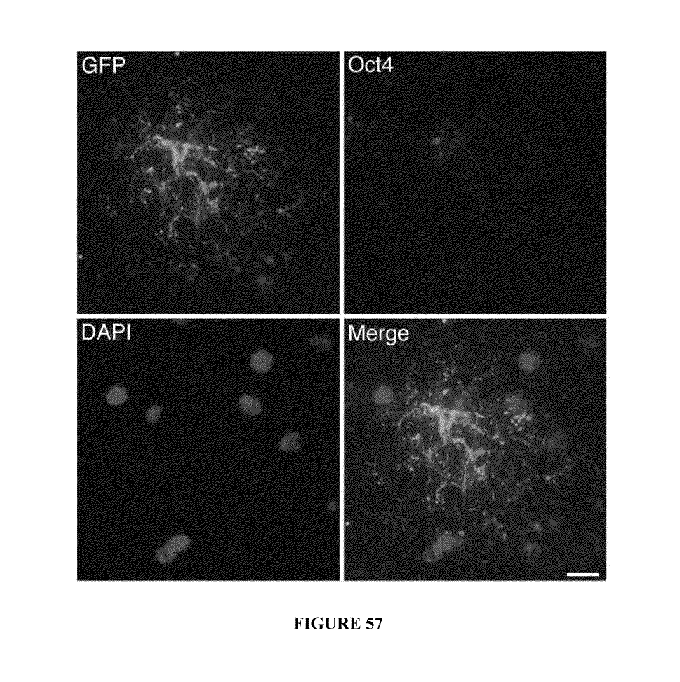

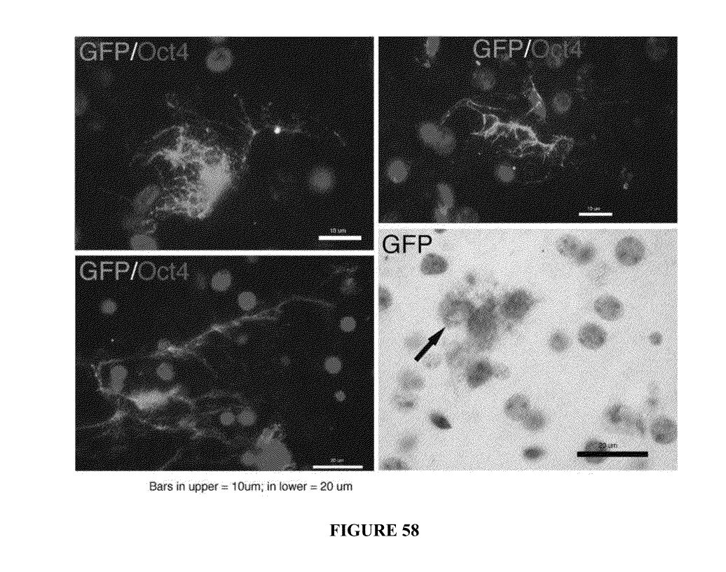

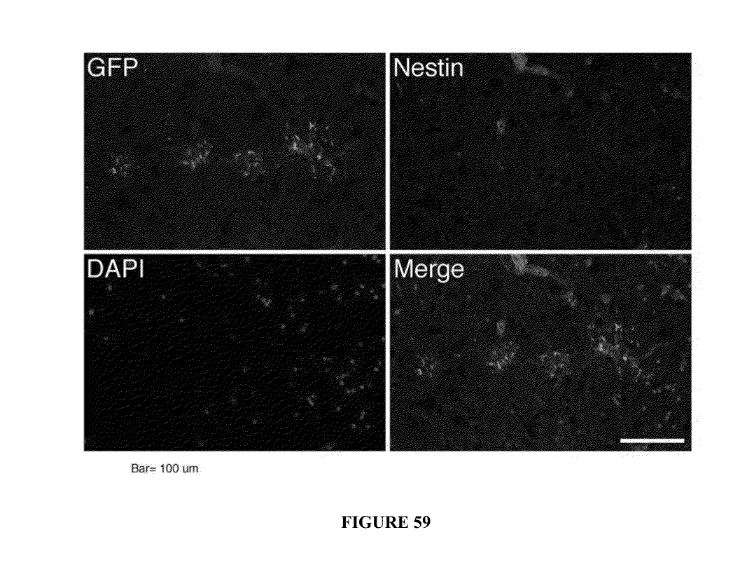

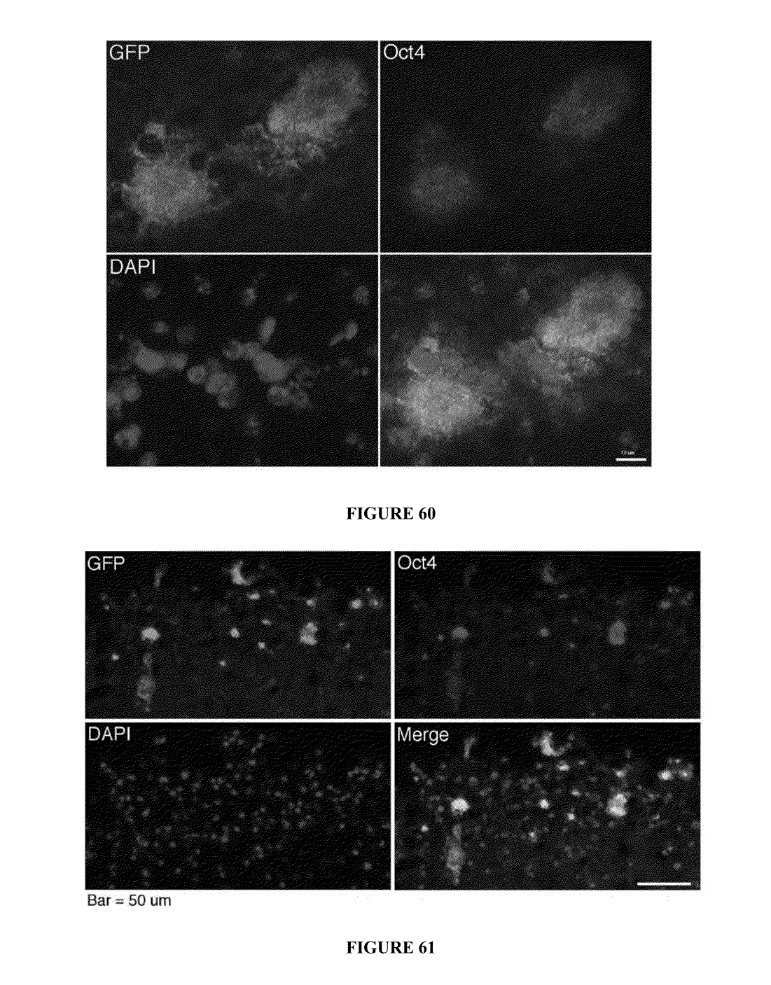

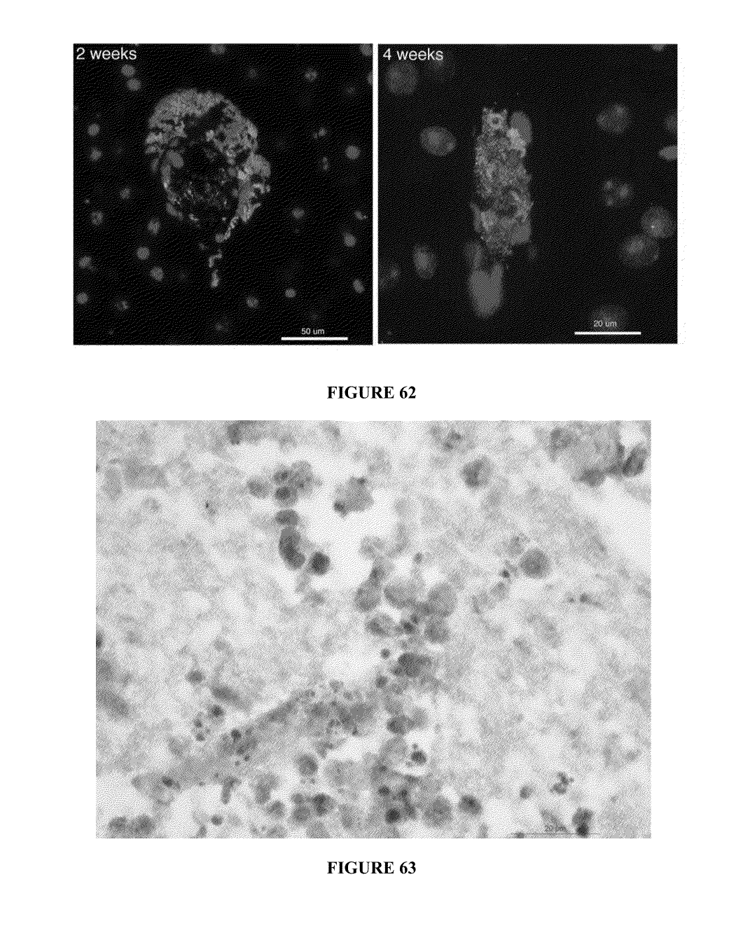

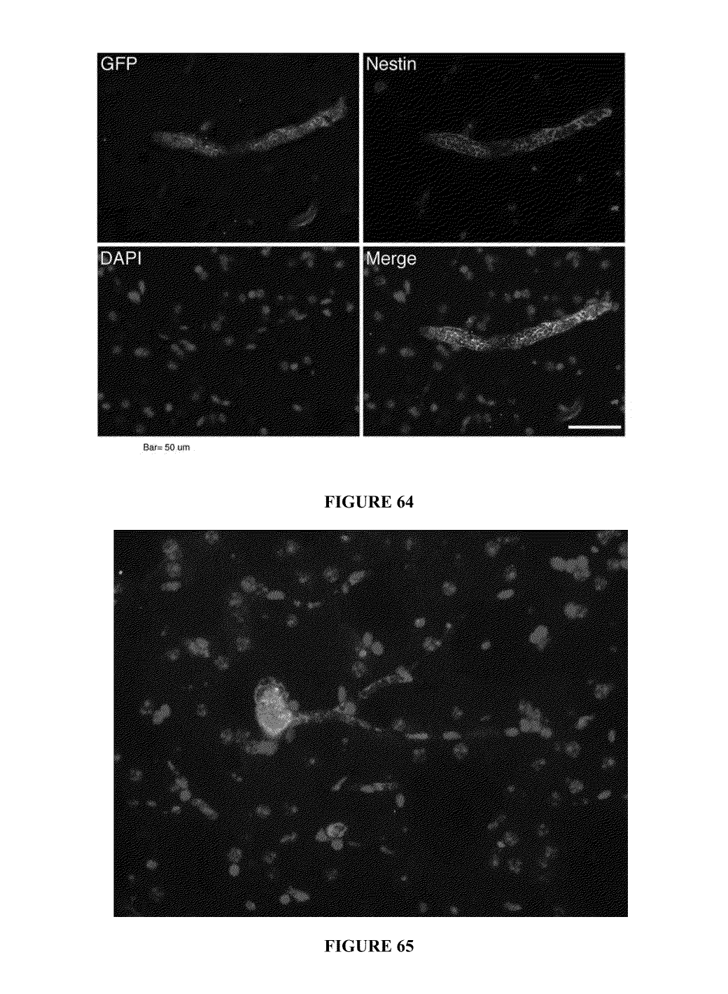

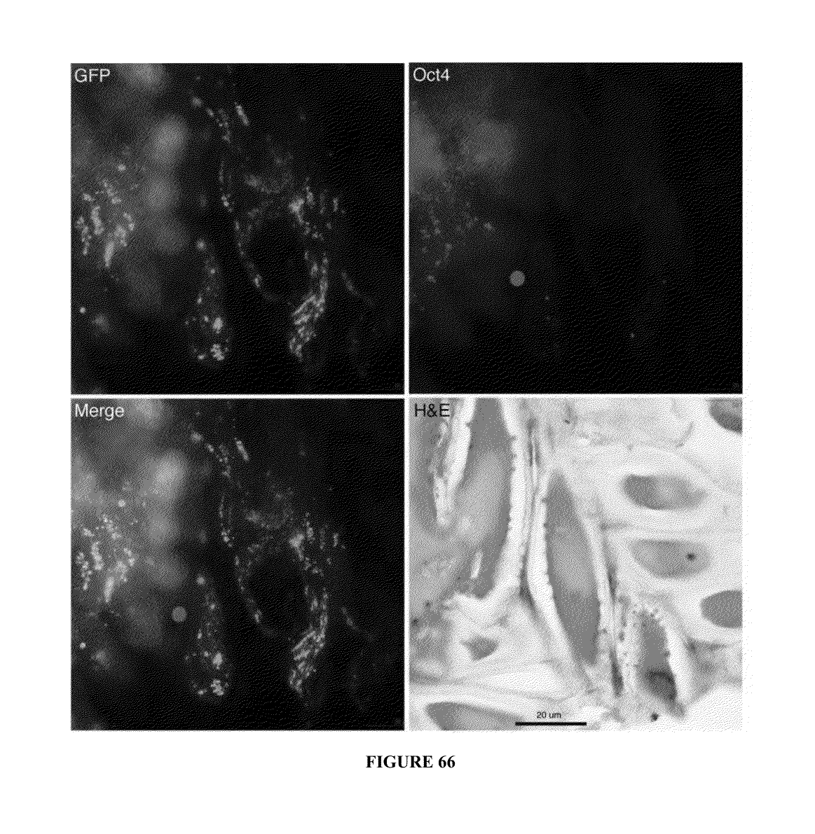

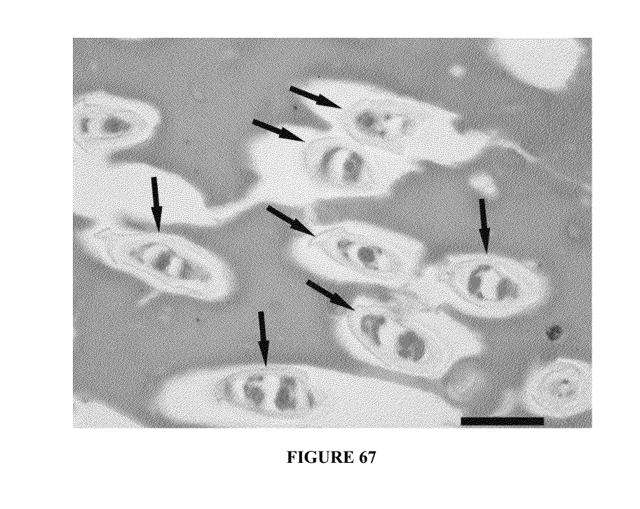

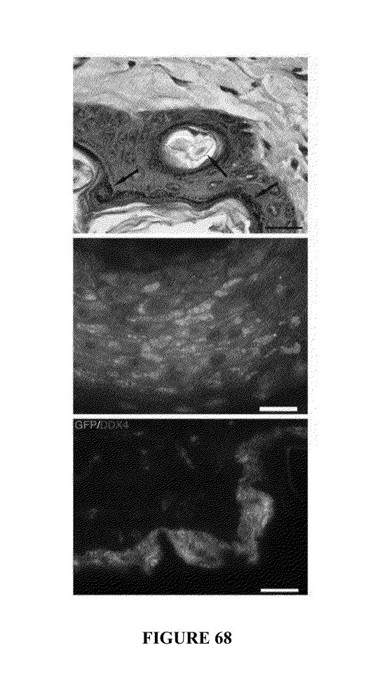

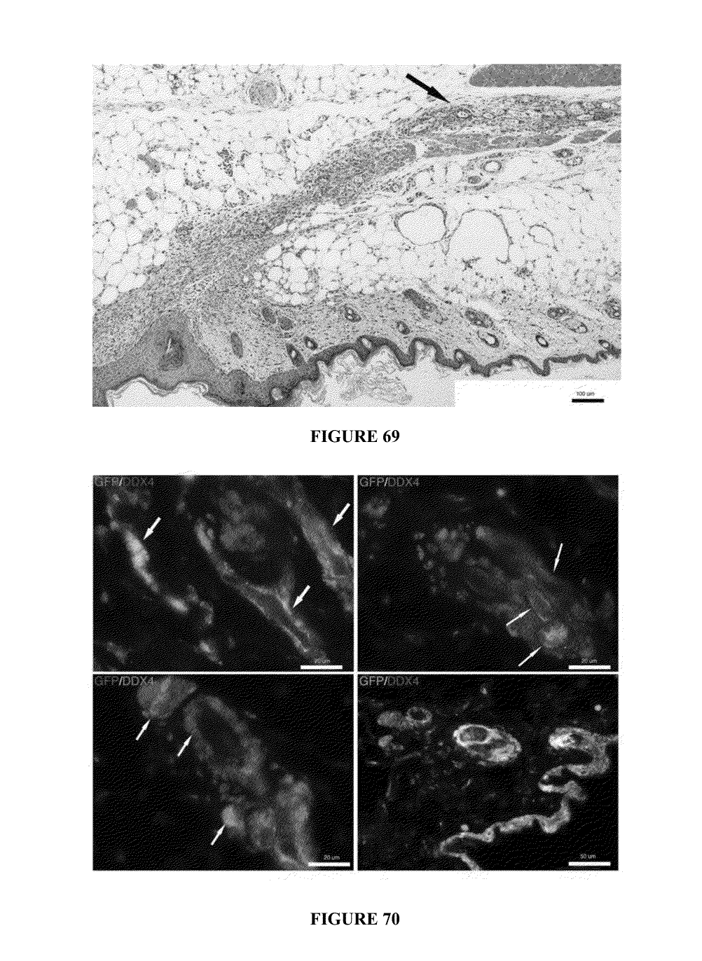

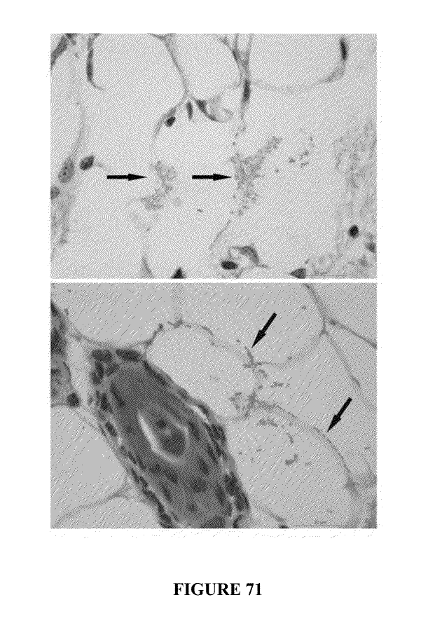

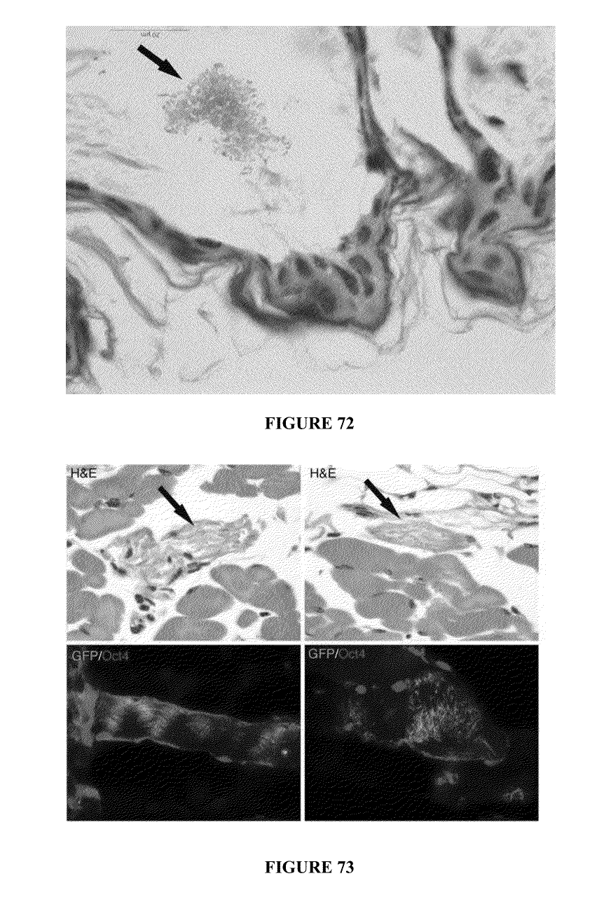

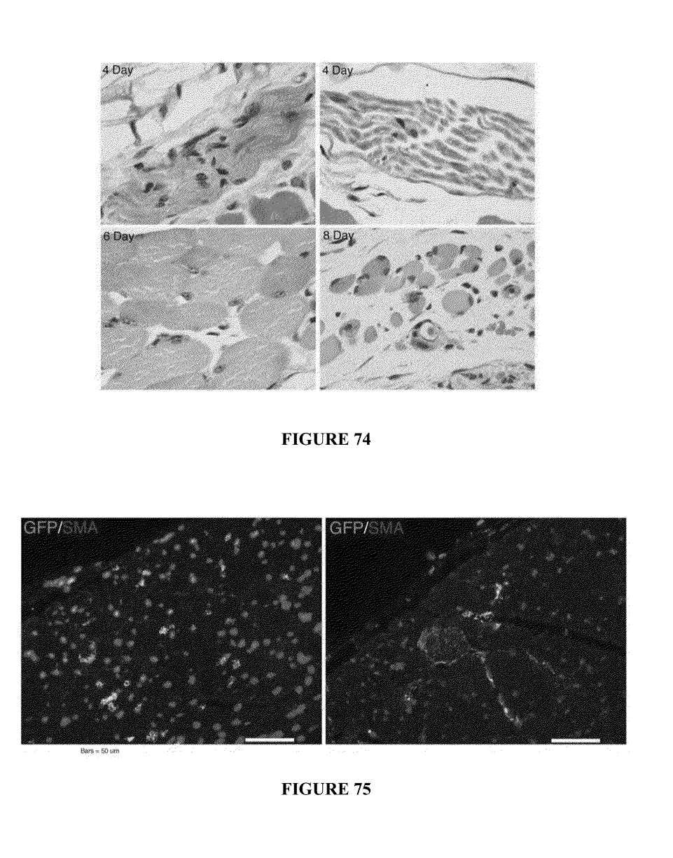

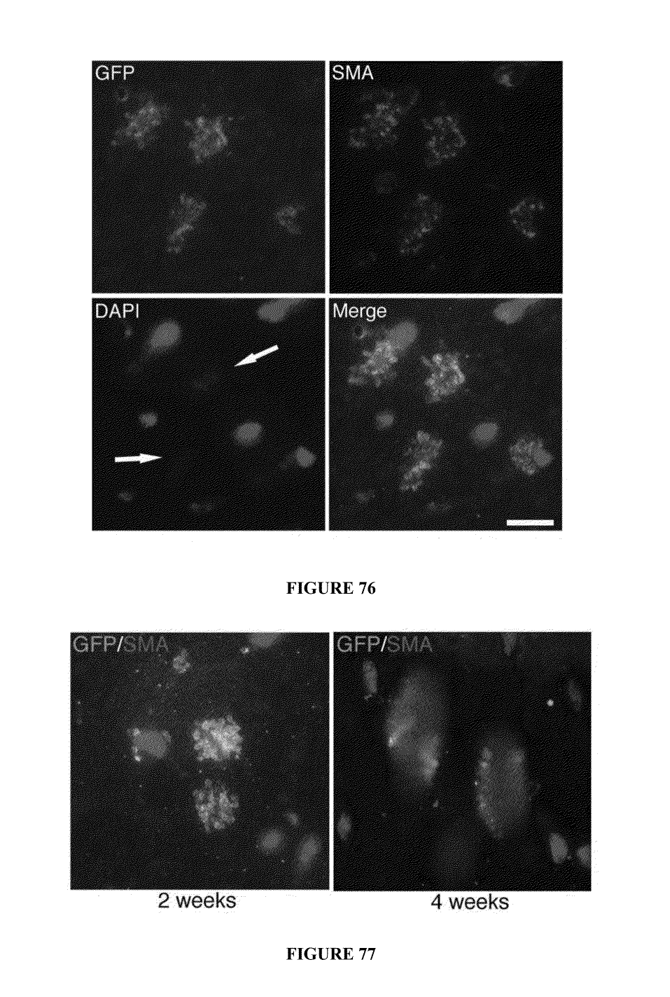

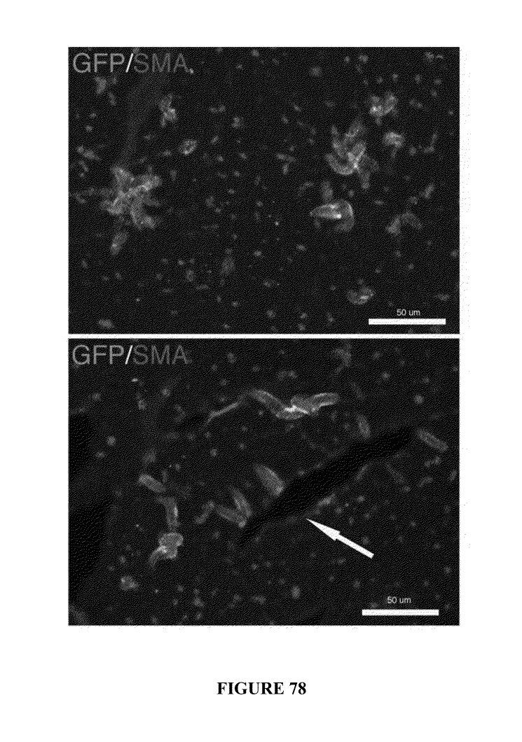

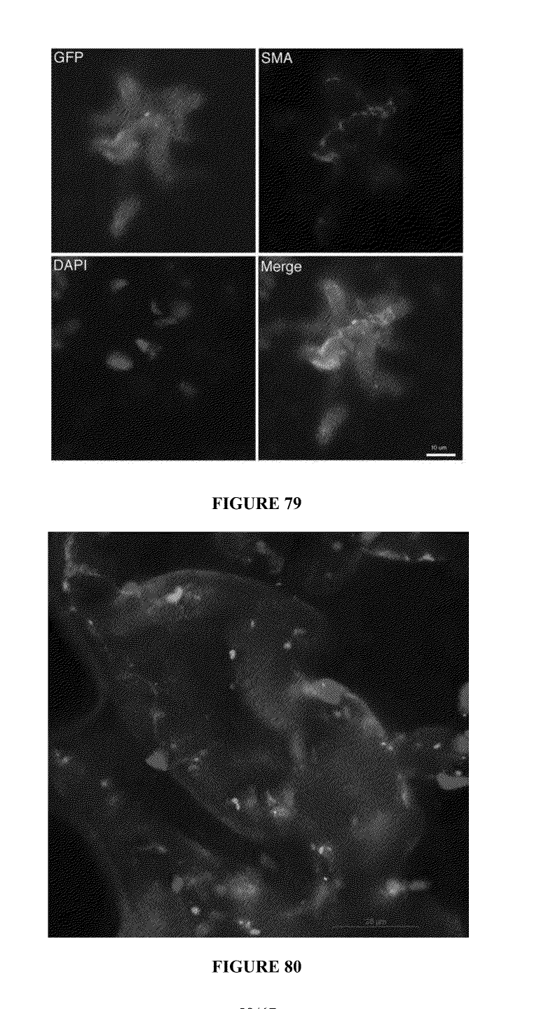

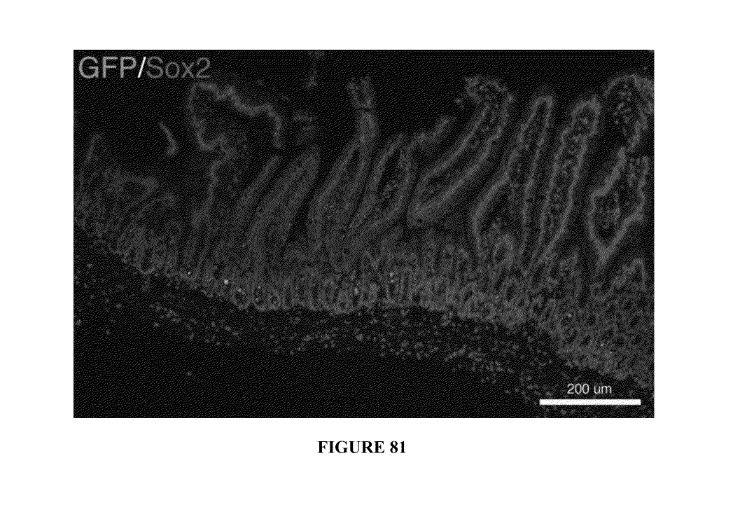

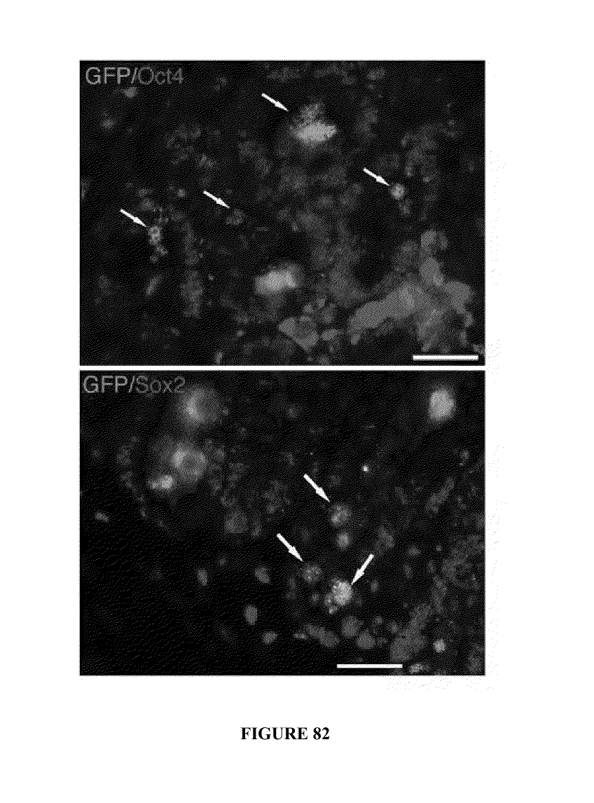

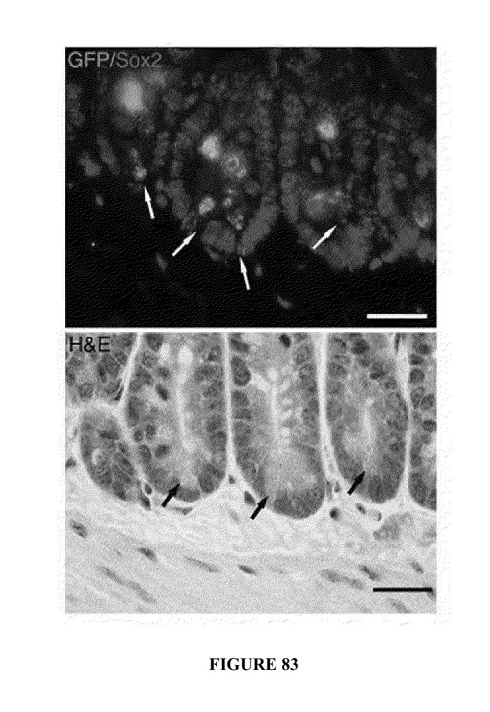

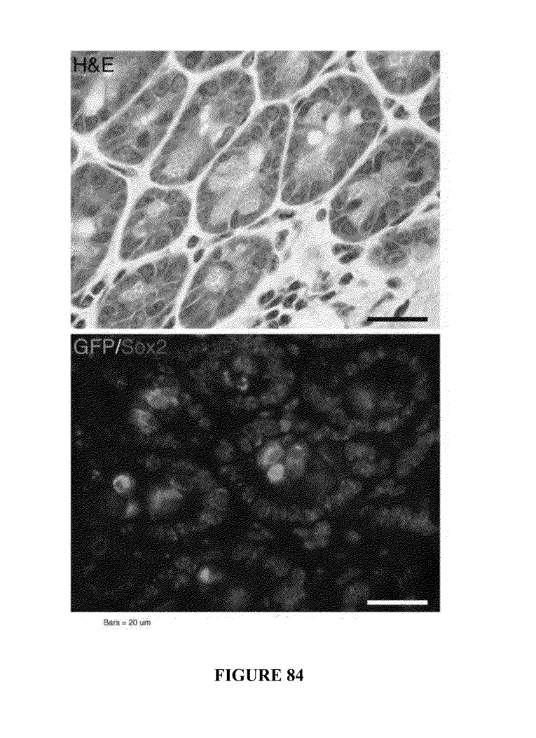

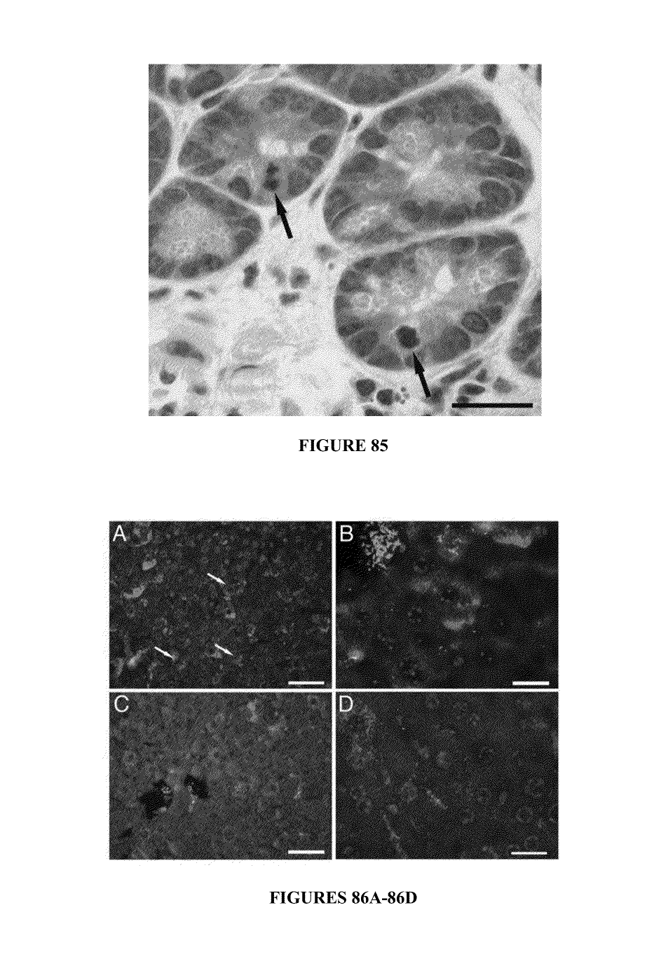

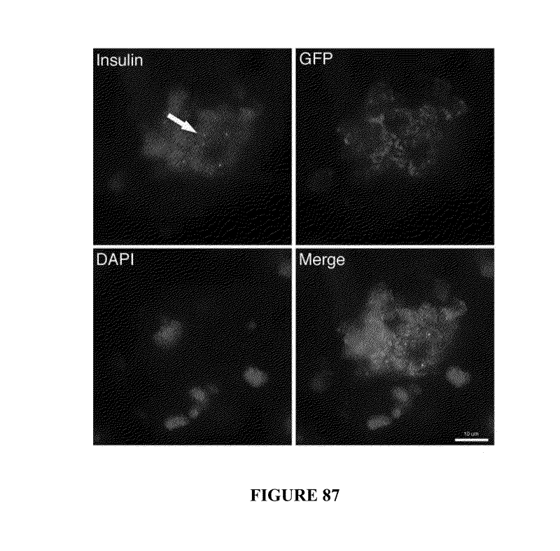

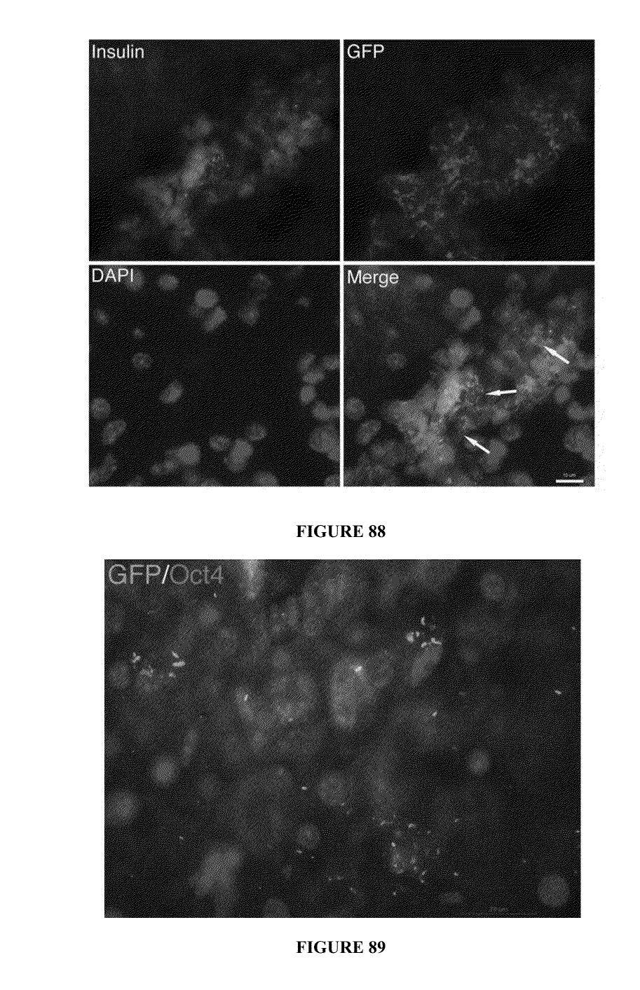

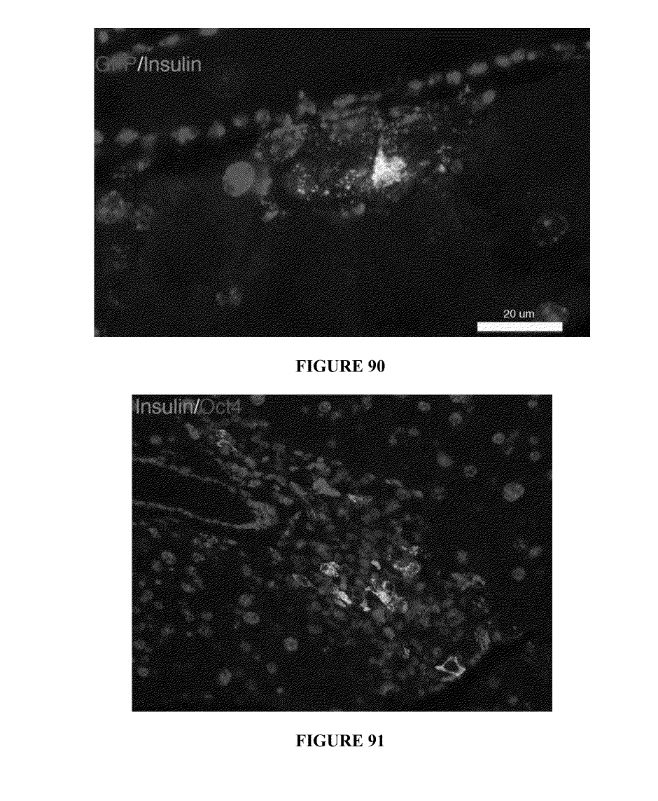

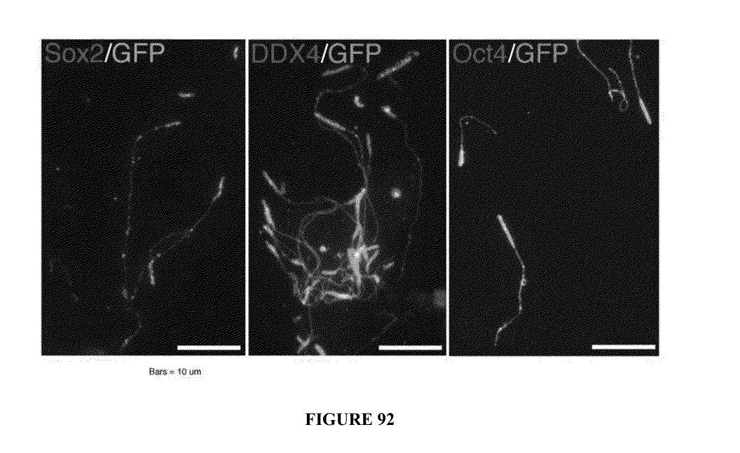

5) The uniform and not condensed type (FIG. 6). This type of NPRCPs contains small nuclear granules and fiber-like structures that distribute evenly inside the components. These NPRCPs are in round shape with thin membranes.

FIG. 7 shows that the NPRCPs contain only less than 200 nt RNA fragments. RNA from NPRCPs and pre-cells were collected and run on an Agilent 2100 Bio-analyzer. The RNA bands were analyzed and the results indicate that NPRCPs have only RNAs that are less than 200 nt. About 30% of these small RNAs are microRNAs. Ribosomal RNAs can be seen in the pre-cell samples. The data was repeatable in three separated samples.

FIGS. 8A-8D show the specific marker expression in the components of the NPRCPs. Oct4 and Sox-2, the embryonic stem cell markers are expressed on about 80% of NPRCPs. In comparison, DDX4/VASA, the germ cell marker is expressed on about 60% of NPRCPs. Sox2 and DDX4/VASA are expressed as particle shapes on NPRCPs, evidencing that their expression is specific. About 40% of them co-express tubulin and all of them express actin. NPRCPs do not stain for DAPI, evidencing that they do not have nucleus, but have small amount nuclear materials. Scale bars=10 μm.

FIG. 9 shows an immunofluorescent stain of the surface markers on NPRCPs. NPRCPs stain positive for c-kit (about 30%), integrin β1 (more than 80%) and E-cadherin (less than 5%). They do not express CD90. Scale bars=10 μm.

FIG. 10 shows an immunofluorescent study of CD45 and CD34 expression. It was found that CD45, the blood stem cell marker is expressed on most small round-shaped particles. In the lower panel, a group of DNA stains for DAPI, although has not become the nucleus, and strongly for CD34, indicating the CD34 expressing group is a pre-cell. Scale bars=10 μm.

FIG. 11 shows cultured human umbilical cord blood-derived NPRCPs which were imaged by a 40× lens using a Nikon microscopy. Images show that NPRCPs increase their sizes by fusion with multiple small particles. Scale bars=10 μm.

FIG. 12 shows in vitro expansion of NPRCPs. Images show that NPRCPs can be enriched during culture. Images were under the same field at day 0 (left) and 5 days later (right). The number of NPRCPs is significantly increased, evidencing that NPP can be enriched in the medium.

FIG. 13 shows that NPRCPs are produced from non-nucleated cells. NPRCPs in human umbilical cord blood cells were isolated and dropped on cover glass and stained for haematoxylin and eosin. Numerous NPRCPs having spindle shapes (left) and round shapes (right) were identified. Both types of NPRCPs locate closely to a larger cellular structure that do not have nucleus (arrows).

FIG. 14 shows that three different types of NPRCPs are identified. The spindle shape (left) and round shape (middle) NPRCPs were identified in human umbilical cord blood. The short-rod shaped NPRCPs (right) were identified in vivo in mouse tissues after transplantation of mouse NPRCPs. All three types of NPRCPs were stained for haematoxylin and eosin. No specific eosin staining was observed, indicating these NPRCPs contain more nuclear materials than proteins. Scale bars=10 μm.

FIG. 15 shows that the NPRCP-producing cells are nucleated after differentiation. NPRCPs were collected from human umbilical cord blood and dropped on cover glass and stained with H&E. A dense haematoxylin stained nucleus (arrow on left) is seen on the left image compare to the non-nucleated cell (arrow on right). These data indicate that these NPRCP-producing cells can be nucleated when they are differentiated. Scale bars=10 μm.

FIG. 16 shows the live images of NPRCP-producing cells. Images were taken in cultured human umbilical cord blood. Numerous small NPRCPs (left) locate around a small cellular structure. Relative larger sized NPRCPs locate near a small cellular structure (right). These data evidence that these cellular structures produce small NPRCPs that further grow extracellularly. Scale bars=10 μm.

FIG. 17 shows that NPRCP-producing cells become nucleated after differentiation. Human umbilical cord blood cells were cultured. Image shows a nucleated cell that is releasing numerous small particles. This cell attaches to the bottom of culture plate and has a clear nucleus. This data indicate that these NPRCP-producing small cellular structures in FIG. 15 can be differentiated, which is characterized by appearing nucleus.

FIG. 18 shows that NPRCP-producing cellular structures express Oct4. Human umbilical cord blood-derived cells were dropped on cover glass and stained for Oct4. The upper panel shows a cellular structure containing numerous small DAPI-dense particles. A small particle is budding off this cellular structure (arrows). The lower four images show a strong oct4-expressing structure that has weak DAPI staining. It shows weak DAPI stain only when light is over exposed. Numerous small oct4- and DAPI-positive particles locate around this structure. These images indicate that NPRCP-producing cellular structures do not have a nucleus at early stages. Scale bars=10 μm.

FIG. 19 shows that NPRCPs fuse to form PFDNCs. Time-lapse images show a group of NPRCPs that fuse into a cellular structure in 11 min. Numerous small vesicles surround the structure. Scale bar=10 μm.

FIG. 20 shows that non-nucleated PFDNCs are developed from at least 2 NPRCPs having the size about 3-10 μm. One particle has a thin membrane which can be seen when they reach maturation (arrow). Scale bars=10 μm.

FIGS. 21A-21F shows the different morphologies of PFDNCs when they are in the culture plates. PFDNCs are characterized by having amoebic-like motion. They twist their bodies in the cultural medium (FIGS. 21A-21F). Images in FIGS. 21G-21I show the differentiated PFDNCs. They have the sizes from 10 to 20 μm. They change their motion to crawling on the plates. Scale bars=10 μn.

FIG. 22 shows the expression and morphology of active integrating DNA and RNA (previously termed Aidars) PFDNCs. Cropped images of inactive (A) and active (B) PFDNCs. H&E (C) and tubulin immunofluorescent staining (D) of PFDNCs for Oct4, SOX2, integrin β1, tubulin, actin and DDX4. The middle 2 panels show immunofluorescent staining of PFDNCs. All merged images were stained with DAPI (blue). Images were taken by upright conventional fluorescent microscopy. Scale bars=10 μm.

FIGS. 23A-23B show that PFDNCs are derived from collection of cytoplasmic material by NPRCPs. Electron microscopy images of 2 pre-cellular structures, each about 5 μm (A) and 8 μm (B) with an NPRCP in the center (arrows), which evidences that the core of PFDNCs are derived from NPRCPs.

FIGS. 24A-24C show the FACS results of PFDNCs. FIGS. 24A, 24B and 24C are the results of the detection of surface markers on PFDNCs by fluorescence activated cell sorter (FACS). FIG. 24A=control group; FIG. 24B=CD29 and FIG. 24C=CXCR4 expression. FACS analyses demonstrate that PFDNCs express more that 60% PFDNC express CD29. About 40% of them express CXCR4. They do not express E-cadherin. The figures in the following are the results from FACS experiment. Results were repeatable after performing other experiments.

FIG. 25 shows that NPRCPs and PFDNCs become nucleated cells. This figure describes the transformation of NPRCPs (upper left image) that are about 1 to 2 μm to the larger sized NPRCPs (upper right image). NPRCPs further fuse with proteins (arrows in lower left image) and become differentiated. The differentiated PFDNCs still do not have typical nucleus compared to the nucleated cells (lower right image). Scale bars=10 μm.

FIG. 26 shows that newly PFDNC-derived cells are stem cells. H&E stains (panels A-D) indicate that PFDNCs do not have strong eosin stains as that of nucleated cells. During PFDNC's differentiation, their protein contents increase while the nucleus is still not formed yet. Immunofluorescent stains indicate that PFDNCs and differentiated PFDNCs stain for Oct4 (Arrow in panel H), however the fully nucleated cells have no Oct4 stains (arrows in lower panel). Scale bars=10 μm.

FIG. 27 shows images of mature PFDNCs that actively get inside other cells to take materials from these cells. Two mature PFDNCs (arrows) are inside the eukaryotic cells at 28 min (thin arrow) and 55 min (thick arrow). After they come out from the nucleated cells, they become larger sized differentiated PFDNCs. Scale bar=50 μm.

FIG. 28 shows images showing that differentiated PFDNCs can transform into eukaryotic cells by fusion to each other and entering the cytoplasmic membrane. This process can occur in a few min. The upper panel shows fusion of two pairs differentiated PFDNCs become two pre-cells. The lower panel shows one pre-cell fuse into a cytoplasmic membrane and become a cell in 9 min. Scale bars=10 μm.

FIG. 29 shows fusion-differentiation of cells by PFDNCs. Time-lapse images show fusion of PFDNCs. Three PFDNCs (arrows) fuse to become a cell within 18 min. Bar=10 μm.

FIG. 30 shows the PFDNC-derived pre-cells have DNA (dense blue in the large one) in the nuclear areas. The dense blue stain in the large pre-cell is not round, suggesting this pre-cell is forming a nucleus. Thin and loose membrane can be seen in all three images, suggesting that they are from the same type particles. Scale bars=5 μm.

FIGS. 31A, 31B show NPRCP fusion-differentiation. Co-cultured NPRCPs were examined by microscopy and video recorded. The time-lapse images were cropped from snapshots of a video record to show a group of small particles fusing into a cellular structure in 150 min (FIG. 31A). Staining for OCT4 expression in co-cultured cells (FIG. 31B). Scale bar in A=20 μm; B=10 μm.

FIG. 32 shows NPRCP fusion-derived large cells release small non-nucleated cells. A live cell (panel A) in the culture plate protrudes at least 2 cellular portions (arrows in A). H&E staining of a large cell (panel B) shows irregular haematoxylin staining. The protruding cytoplasmic portions show slight haematoxylin staining at the outer edges (arrow in B). Immunofluorescence staining of protruding portions shows a large amount of tubulin and OCT4 and SOX2 expression (arrows in C and D). Live cell releases a small non-nucleated call and shows 2 more protrusions (arrows in E). Images in the lower panel show time-lapse of a cellular protrusion (arrows) with extremely active motion. Scale bars in A-E=10 μm, lower panel=20 μm.

FIGS. 33A-33C show that multiple NPRCPs are fused to form nucleus (FIGS. 33A, 33B). DAPI stains on irregular shaped nucleus. The images indicate that DNA appears on multiple NPRCPs, which locate close to each other. DNA becomes a nucleus after rearrange or nuclear programming. Bar in B=5 μm. Large fused cellular structures differentiate into multiple cells (FIG. 33C). These cells are located in the large structure that express strongly to oct4 (red). No clear cytoplasmic components were observed. Bar in C=10 μm.

FIG. 34 shows evidence of nuclear programming in PFDNC-derived pre-cells. NPRCPs and their aggregated products, PFDNCs, do not have DNA or nucleus. They collect DNA from the nucleus of the eukaryotic cells or from circulating DNA fragments and then undergo nuclear programming. Images show the pre-cell with no DNA (left in upper panel), scattered DNA (left in lower panel) or circle-like form DNA (lower right), indicating that they are undergoing nuclear programming to become eukaryotic cells. Scale bars=10 μm.

FIG. 35 shows evidence that other types of cells can be formed by NPRCP assembly. Cultural Enriched NPRCPs are stained for H&E. Upper images show two H&E stain cellular structure that do not have aggregated particles, suggesting they can transform into other type of cells, but not mesenchymal-like cells. Scale bars=5 μm. Lower images show the electron microscopy of the NPRCPs that are direct transform into one type of blood cells. These particles exhibit a small round shape and the dense center fads as they become larger. No nucleus is seen in these particles, indicating that they are not cells.

FIGS. 36A, 36B show the isolation of purified NPRCPs. NPRCPs are different from PFDNCs by their sizes and their motions. The purified NPRCPs (FIG. 36A) and the mixture of PFDNCs, differentiated PFDNC and pre-cells (FIG. 36B) are shown. These evidence the successful implementation of the methods embodied herein for isolating and purifying NPRCPs.

FIGS. 37A-37J shows the morphology and expression of mouse non-platelet RNA-containing particles (NPRCPs) by inverted microscopy at day 2 (FIG. 37A) and week 2 (FIG. 37B) after culture. NPRCPs cultured on cover slides for 20 days were fixed and stained with haematoxylin and eosin (H&E) (FIG. 37C) and green fluorescent protein (GFP) (FIG. 37D). High magnification of NPRCPs after day 2 (FIG. 37E) and week 2 (FIG. 37F) of culture and H&E staining (FIG. 37G). Electron microscope images (H-J) show different morphologies with dense structured in the center (arrows). Scale bars in A-D=20 μm; E-G=10 μm; H-J=500 nm.

FIG. 38 shows NPRCPs derived from GFP-transgenic mice cultured for 3 weeks. Immunofluorescent staining of NPRCPs expressing GFP, Oct4, sox2, DDX4, actin and tubulin. GFP was expressed on the surface and in the center of NPRCPs. Oct4, sox2, and DDX4 were expressed in the center of NPRCPs, and actin and tubulin were expressed mainly on the surface. Scale bars=10 μm.

FIGS. 39A-39G are images showing that NPRCPs migrate to the mouse ischemic kidney at day 1 after transplantation. Kidney staining by H&E (FIG. 39A), GFP-immunohistochemistry (FIG. 39B) and immunofluorescence (FIGS. 39C and 39D). High-magnification H&E (FIG. 39E) and GFP-immunohistochemistry (FIGS. 39F and 39G) images of extravasated material with GFP-positive staining (narrow-head arrows). Tiny particles and extravasated materials are grouped together (wide-head arrows). Scale bars in C and D=200 μm and E-G=20 μm.

FIG. 40 shows the trafficking of NPRCPs in vivo. GFP-stained NPRCPs were detected by immunofluorescence (A) and immunohistochemical staining (B) in renal calyces areas at day 1 after transplantation into mice with ischemia-damaged kidneys. High magnification of aggregated GFP-positive NPRCPs by immunohistochemical (C) immunofluorescence (D) and H&E staining (E). Tiny dots were detected inside NPRCPs (arrows in C and E). High magnification of NPRCPs show short rod-shaped GFP-positive NPRCPs co-expressing Oct4, sox2, and DDX4 (lower panel). Scale bars=10 μm.

FIGS. 41A-41D show that extravasated NPRCPs aggregate (arrows in FIGS. 41A and 41B) and fuse into non-nucleated cellular structure (arrows in FIGS. 41C and 41D) on the first day after the transplantation. Scale bars=20 μm.

FIGS. 42A-42J show three regenerative patterns. NPRCPs were found in small groups of aggregates in a glomerulus (broken line, FIG. 42A), large aggregations in the tubule area (FIG. 42B), or lined up in the interstitial areas (FIG. 42C) in the kidneys, or in the large tubule (FIG. 42D) and in a blood vessel in the renal capsule (FIG. 42E) at day 1 after transplantation in mice with ischemia-damaged kidneys. Haematoxylin-stained (FIG. 42F) and GFP-positive (FIG. 42G) tiny nucleated particles were found in connective tissues. Aggregated NPRCPs co-expressed DDX4 (FIG. 42H), Oct4 (FIG. 42I), and sox2 (FIG. 42J). Scale bars in A-D=20 μm and in E-J=10 μm.

FIGS. 43A, 43B show that GFP-positive NPRCPs fuse into large patch-like structures at weeks 1 (FIG. 43A) and 2 (FIG. 43B) after transplantation into mice with ischemia-damaged kidneys. The fused patches also co-express sox2. Scale bars in upper image=100 μm and lower image=50 μm.

FIGS. 44A-44F show renal tubule regeneration at early stages. H&E staining of large cellular structures containing central nuclear materials (arrows in FIGS. 44A and 44B). GFP fluorescent further confirmed that these large cellular structures were formed by the fusion of NPRCPs (arrows in FIGS. 44C and 44D). GFP-positive large tubules were co-expressed with Oct4 (FIG. 44E). No nuclei detected by DAPI (FIG. 44E) or H&E staining (FIG. 44F). Scale bars in A-C=10 μm and in D-F=20 μm.

FIG. 45 shows interstitial tissue regeneration. Tiny cells co-expressed GFP (arrows) and DDX4 in the interstitial areas at 1 week after transplantation in mice with ischemia-damaged kidneys. High magnification of DAPI (box in DAPI frame) and H&E staining showing tiny nuclei in GFP-positive cells (arrows). Scale bars in merge=20 μm and in H&E=10 μm.

FIGS. 46A-46H show glomerulus regeneration. (FIG. 46A) Week 1 after transplantation in mice with ischemia-damaged kidneys. GFP and DDX4 are co-expressed as small particles in a non-nuclear area (circle in DAPI frame). (FIG. 46B) Week 2 after transplantation. GFP-positive glomerulus co-expressed Oct4. DAPI staining shows no nuclei (circle in DAPI frame). H&E staining shows possible regenerative stages of glomeruli from day 1 to week 4 (FIGS. 46C-46H; tiny nuclei with arrows). Scale bars=10 μm.

FIG. 47 shows glomeruli are regenerated by NPRCP. A glomeruli capillary structure is formed by the connection of GFP-positive (use ABC stain) NPRCPs. No nucleus is seen. A few GFP-positive small cells are seen nearby. Scale bar=10 μm.

FIG. 48 shows the regeneration of interstitial cells (green arrows) and renal tubule cells (black arrows) from day 1 to week 4 after NPRCP transplantation. The regenerated tubules show brush border structures positive for GFP (circles). Scale bars=20 μm.

FIGS. 49A-49D show that tubule regeneration occurs later in medulla than in cortex. At week 1 after transplantation, distal tubules are filled with numerous extravasated NPRCPs (FIG. 49A) that fuse into large cellular structures with a tiny dot (arrows in FIG. 49B). At week 4, the duct tubules in the renal medulla are regenerated by extravasated structures (FIG. 49C), which have different morphologic features and contain a tiny dot under high magnification (arrows in FIG. 49D) and suggest endogenous NPRCPs. Bars in A=50 μm; in B=10 μm; in C=20 μm.

FIG. 50 shows kidney regeneration during 4 weeks after NPRCP transplantation in mice with ischemia-damaged kidneys. At day 1 and week 1 after transplantation, kidneys show more than two-thirds necrotic areas. At week 2, kidneys show about one-third necrotic areas. At week 3, kidneys show regenerated cortex but not medulla area. At week 4, kidneys show regeneration in most areas. These images were merged from images taken by a 2.5× lens.

FIG. 51 shows the appearances of GFP-expressing NPRCPs in the ischemic damages brains 4 days after transplantation. GFP was stained using DAB methods. The tiny brown color particles appeared in the brain 4 days later.

FIG. 52 shows an image from immunofluorescent microscopy indicating that, similar to the kidneys, numerous NPRCPs are aggregated in the damaged areas in the brain. Small NPRCPs express both GFP and nestin, the neuron stem cell marker. However, GFP is stained as vesicle, while nestin is expressed as small dots.

FIG. 53 shows that aggregated NPRCPs form into new neurons. The nuclear materials in the aggregated GFP-expressing NPRCPs fuse, undergo nuclear programming, and become the nucleus of the new neuron. Two nuclei (arrows in DAPI) were enlarged (the lower images). It clear shows that these two nuclei are formed by small dots-shaped DAPI-positive particles. In addition, GFP-expressing NPRCPs are express nestin, indicating they form into neuron stem cells.

FIG. 54 shows that 6 days after transplantation, most NPRCPs have lost their small vesicles morphology and have fused into larger grouped structures in brain.

FIG. 55 shows the appearance of dendrites 7 days after transplantation. The aggregated NPRCPs start to protrude the dendrites on the 7 days after the transplantation. Meanwhile, dots-shaped nestin has linked together and become fiber-like structures.

FIG. 56 shows the mechanism of dendrite formation (early stage). Images show NPRCPs (GFP) scattered in a brain area 5 days after transplantation. At early stage, NPRCPs are scattered in certain areas and form into a round shape. Nestin is expressed as tiny dots and also scattered in the same area.

FIG. 57 shows the mechanism of dendrite formation (middle stage). NPRCPs protrude fine fibers that connect to each other to form dendrites. Images show GFP-expressing NPRCPs that have lost the small vesicle shape and become either dot or line shapes. Oct4 is expressed in the core area of this GFP-expressing structure, suggesting which is going to be the newly formed nucleus. These data also indicate the newly formed cell is a stem cell. Scale is =10 μm.

FIG. 58 shows the mechanism of dendrite formation (end stage). Dendrites are formed before the nucleus is fully formed. Immunofluorescent and immunohistochemistry images further confirm that the dendrites of the new neurons are formed by the connection of NPRCPs and further differentiation. A nucleus can be seen in the DAB stained image (arrow, lower right), which stains weak to haematoxylin compared to other nuclei.

FIG. 59 shows newly formed neurons with dendrites in the damaged brain areas. The lower magnified images indicate that three dendrite-expressing neurons that were lined up, locate in the areas having fewer brain cells, or fewer nuclei. This data indicates that NPRCPs can form into dendrite-expressing cells that line up in the damaged brain areas.

FIG. 60 shows the mechanism of forming axons. Besides forming dendrites, NPRCPs also aggregate to form axons. However, axon formation appears slower and also in different patterns. The aggregated NPRCPs are surrounded by a layer of nucleated cells. Images show aggregated NPRCPs that also express oct4 on 12 days after transplantation.

FIG. 61 shows axon formation (early stage). Aggregated NPRCPs that are surrounded by nuclei are shown under lower magnification. These aggregates express oct4.

FIG. 62 shows axon-formation (early stage). Aggregated NPRCPs have fused into cellular structures, however, without nuclei formation. They are surrounded by a layer of nucleated cells. Image on the left were taken from 2 weeks after transplantation. Images on the right were taken in brain at 4 weeks after transplantation.

FIG. 63 shows that GFP-expressing cellular structures appear in the damaged brains. Except for small haematoxylin-stained small nuclei, most of these GFP-expressing cellular structures do not have a nucleus, indicating that these are from NPRCPs and undergoing nuclear formation.

FIG. 64 shows that axon formation appeared by 10 weeks after NPRCP transplantation. Images show GFP and nestin are co-expressed on an axon-like structure 10 weeks after NPRCP transplantation. This data indicate that NPRCPs can regenerate axon, however, in a slower procedure.

FIG. 65 shows that the axon-expressing neuron is GFP-positive. Immunofluorescent images show that the oct4 (red)-expressing cells have long axons and also GFP positive at 10 weeks after transplantation.

FIG. 66 show that NPRCPs form into hair bulbs. Images show 2-day wound stained to GFP Oct4 and H&E. NPRCPs were extravasated at the wound edge with the shape of hair bulbs.

FIG. 67 show that hair bulbs have formed before NPRCPs migrate to the wounds. Hair bulb-shaped non-nucleated cells that are wrapped in hyaline layer are embedded in the plasma at the wound edge, indicating that these hair bulbs are extravasated. Bar=20 μm.

FIG. 68 shows NPRCPs regenerate epithelial. Upper and middle images shows the H&E stain, Oct4 and GFP expression of 4 days wound. 4 days after wounds, NPRCPs appear at the epithelial layer, not at the basal layer but at the surface layer (arrows). A non-nucleated cell that wrapped in hyaline layer is surrounded by NPRCPs. Immunofluorescent data indicate that these small NPRCPs in the epithelial express Oct4. They locate at the areas where do not have nuclei. Lower image shows a 6-day wound. Newly re-epithelialized skin layer was stained to GFP and DDX4. DDX4 stained mainly on the peripheral areas of the nuclei, indicating these nuclei are undergoing nuclear programming. Bars=20 um.

FIG. 69 shows that NPRCPs form hair bulbs before reaching the epithelial layer. Image show a merged 6 day wound sections. Skin wound has re-epithelialized, however hair follicles have not fully regenerated. New hair bulbs (arrow) are migrating to the epithelia area, suggesting the hair follicles are undergoing regeneration.

FIG. 70 show that the epithelial layer and hair follicles are regenerated by NPRCPs. New hair follicles express GFP and DDX4 in 4 day wound sections, supporting these hair follicles are derived from NPRCPs.

FIG. 71 shows that skin connective tissues are regenerated by NPRCPs. Grouped NPRCPs locate in the connective network (arrows in upper image) in 8 day wounds, indicating that they aggregate the fuse into the new nuclei. Small NPRCPs that stain weak to hematoxylin is line up (arrows in lower image) to form the connective network structures.

FIG. 72 shows that NPRCPs regenerate connective tissues. Aggregated NPRCPs (arrow) locate in the damaged dermal area. This image shows that NPRCPs regenerate the dermis.

FIG. 73 shows that NPRCPs regenerate smooth muscle. Images show 2-day wound smooth muscle areas. Small non-nucleated cells that line up to form a larger structure (arrows in upper images). Lower images indicate that newly formed muscle fibers express both oct4 and GFP, supporting that these muscle fibers are derived from NPRCPs.

FIG. 74 shows images of the process of smooth muscle formation. At 4 days, non-nucleated cells line up to form the pre-muscle masses. By day 6, the newly formed smooth muscle fibers are loose with large nuclei at the outer edges of the muscle fibers. By day 8, some myoblasts show large nuclei and irregular shapes.

FIG. 75 show that NPRCPs migrate into heart tissues after transplantation. NPRCPs migrate into heart tissues after transplantation. Images show 2 weeks heart sections. Small grouped GFP-expressing NPRCPs appeared in the heart tissue (left image) and in blood vessel (right image).

FIG. 76 shows that NPRCPs aggregate into groups after migrating into heart tissues. 2 week heart sections. Grouped NPRCPs fuse into cardioblasts that also express smooth muscle actin, the stem cell marker. Smooth muscle actin stains as particle-shape, not fiber-like, indicating the fiber-shape is expressed on the differentiated cells. DAPI stain indicates that the newly fused NPRCPs are forming nuclei (arrows). Bar=10 μm.

FIG. 77 shows that aggregated NPRCPs form into cardiac myoblasts. GFP-expressing NPRCPs aggregate at 2-week ischemic heart. No aggregated NPRCPs were detected, instead, only GFP-expressing cardioblasts are found in 4-week hearts. Smooth muscle actin can be detected.

FIG. 78 shows circulation-derived NPRCPs form into cardioblasts. Images show newly formed cardioblasts in hearts 8 weeks after transplantation. They form into grouped or scattered myoblasts. Some of them locate right adjacent the blood vessel (arrow in the lower image).

FIG. 79 show that NPRCPs form into cardiac myoblasts. Higher magnified images show a group of newly formed GFP-expressing cardiac myoblastes at 8 weeks after NPRCP transplantation. A couple of GFP-expressing NPRCPs can still be seen in the newly formed myoblasts.

FIG. 80 show that NPRCPs form into cardiac myoblasts. Higher magnified mages show a group of newly formed GFP-expressing cardiac myoblastes at 8 weeks after NPRCP transplantation. GFP-expressing NPRCPs can still be seen in the newly formed myoblasts.

FIG. 81 shows that NPRCPs regenerate intestinal epithelial cells. It is well known that the intestinal epithelial cells at the tip of villa are shed off, while the cells at the bottom, or crypts areas migrate up to fill the spaces. NPRCPs were transplanted via tail-vein injection. Small intestine was collected 7 days after NPRCP transplantation. Under lower magnification, aggregated GFP-expressing NPRCPs were found only at the crypt areas.

FIG. 82 show that NPRCPs migrate to the intestinal crypt and fuse into the cells. Higher magnified images show that Oct4 does not colocalize to NPRCP (arrows in the upper image), while sox2 (arrows in the lower image) co-localizes to NPRCP only at the aggregation stage, but not at the fusion stage.

FIG. 83 shows that NPRCPs migrate to intestinal crypt and fuse into the cells. Higher magnified images show that Oct4 does not colocalize to NPRCP (arrows in the upper image), while sox2 (arrows in the lower image) co-localizes to NPRCP only at the aggregation stage, but not at the fusion stage.

FIG. 84 show that NPRCPs form into crypt cells. Images show the horizontal cross section of the intestinal crypts under higher magnification by H&E stains (upper image) and immunofluorescent stains (lower image). All GFP-positive fused structures are the aggregated NPRCPs, indicating that crypts cells are derived from blood-derived NPRCPs.

FIG. 85 shows the high proliferative rate of newly NPRCP-formed crypt cells. Image shows two newly formed cells with strong haematoxylin stains undergoing mitotic division. These two cells locate at the areas where the NPRCP aggregates are, indicating that they are derived from NPRCP fusion. Bar=20 μm.

FIGS. 86A-86D show regeneration of hepatocytes by NPRCPs. Grouped GFP-expressing NPRCPs (arrows) were found in the liver 4 days after transplantation (FIG. 86A). Some of these grouped NPRCPs are also express Oct4 (red). Under higher magnification (FIG. 86B), scattered GFP-expressing NPRCPs were also identified. Image in FIG. 86C shows scattered GFP-expressing NPRCPs. On day 7 after transplantation (FIG. 86D), higher magnified image shows that GFP-expressing NPRCPs are grouped together or surround to the nucleus. This data does not exclude that NPRCP have the function of induce the differentiated cells to undergo the nuclear programming and become stem cells. However, the mechanism of induced stem cells by NPRCPs is not by modify gene but may be via regulating the gene expression. Bars in A and C=50 um; in B and D=20 um.

FIG. 87 shows that insulin-producing cells are derived from blood. NPRCPs were transplanted into diabetic mice. 2 days after transplantation, GFP-expressing NPRCPs were found as grouped in the pancreatic tissues. Insulin stained as fine particles in the GFP-expressing NPRCPs. A hollow structure can be seen in the center of aggregated NPRCPs, indicating that the nucleus of this cell has not formed yet. This data also suggest that insulin is expressed in the fully differentiated cells, while GFP is expressed before the cells are differentiated. There is a time gap between insulin and GFP expression.

FIG. 88 shows NPRCPs transplanted into diabetic mice. 2 days after transplantation, GFP-expressing NPRCPs were found as grouped in the pancreatic tissues. Insulin stained as fine particles in the GFP-expressing NPRCPs. The hollow structure can be seen in the center of aggregated NPRCPs, indicating that the nucleus of this cell has not formed yet. This data also suggest that insulin is expressed in the fully differentiated cells, while GFP is expressed before the cells are differentiated. This data also support that there is a time gap between insulin and GFP expression.

FIG. 89 shows that scattered GFP-expressing NPRCPs were found in the pancreas 2 days after transplantation. These NPRCPs locate closely with Oct4-expressing particles, suggesting NPRCPs regenerate pancreatic acina cells.

FIG. 90 shows NPRCPs forming new pancreatic islands. A small pancreatic island was detected in the pancreas two month after twice NPRCP transplantation. GFP (red)-expressing NPRCPs locate at the outer edge of the grouped cells, suggesting GFP is fading. 2 cells in the center is expressing insulin that is expressed as particles, indicating they are new insulin-producing cells.

FIG. 91 shows that since the expression GFP and insulin on the cells has a time gap, Oct4 expression was further studied on the pancreatic island cells. Again, Oct4 is expressed on the cells at the edge of the island, while the insulin-expressing cells are not co-localizing with the oct4-expressing cells. These data indicate that pancreatic B cells are not stem cells. These cells are fully differentiated and do not have co-expression of stem cell markers.

FIG. 92 shows that NPRCPs or PFDNCs may release the transcription factors to regulate cell reprogramming in normal conditions. Long-tailed NPRCPs or PFDNCs were found in vivo. They express sox2, Oct4 and DDX4, however with a long tail. This morphology evidences that NPRCPs or PFDNCs release the transcription factors or other regulatory factor that reprogram the nucleated cells. Since PFDNCs can easily get inside the nucleated cells, they can release these regulatory factors directly into the nucleus.

### DETAILED DESCRIPTION

Stem cells induce rejection when used for cell therapy, and therefore blood from both donor and recipient has to be tested for tissue matching, which makes it difficult to find the right donor that have the matched tissue type. In embodiments of this invention, a group of mixed particles termed “non-platelet RNA-containing particles” (NPRCPs) were isolated and cultural enriched. NPRCPs do not have a nucleus and cytoplasmic membranes. Therefore, NPRCPs do not express the antigenic proteins that induce rejection. In addition, NPRCPs can produce a group of cellular structures (PFDNCs) that collect the genetic materials from eukaryotic cells and further transform into this type of eukaryotic cells or mesenchymal stem cells (MSCs). Besides these types, NPRCPs also produce other type of pre-cells that further transform into other blood stem cells or hematopoietic stem cells (HSCs). Because of these benefits, NPRCPs can be used more safely and efficiently in therapeutic treatment than that of MSCs and HSCs. In further embodiments, techniques to purify and further enrich NPRCPs are provided.

The present invention is described with reference to the attached figures, wherein like reference numerals are used throughout the figures to designate similar or equivalent elements. The figures are not drawn to scale and they are provided merely to illustrate the instant invention. Several aspects of the invention are described below with reference to example applications for illustration. It should be understood that numerous specific details, relationships, and methods are set forth to provide a full understanding of the invention. One having ordinary skill in the relevant art, however, will readily recognize that the invention can be practiced without one or more of the specific details or with other methods. The present invention is not limited by the illustrated ordering of acts or events, as some acts may occur in different orders and/or concurrently with other acts or events. Furthermore, not all illustrated acts or events are required to implement a methodology in accordance with the present invention.

Embodiments of the invention may be practiced without the theoretical aspects presented. Moreover, the theoretical aspects are presented with the understanding that Applicants do not seek to be bound by the theory presented.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

### DEFINITIONS

The terminology used herein is for the purpose of describing particular embodiments only and is not intended to be limiting of the invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

The terms “patient” or “individual” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters; and primates.

“Diagnostic” or “diagnosed” means identifying the presence or nature of a pathologic condition. Diagnostic methods differ in their sensitivity and specificity. The “sensitivity” of a diagnostic assay is the percentage of diseased individuals who test positive (percent of “true positives”). Diseased individuals not detected by the assay are “false negatives.” Subjects who are not diseased and who test negative in the assay, are termed “true negatives.” The “specificity” of a diagnostic assay is 1 minus the false positive rate, where the “false positive” rate is defined as the proportion of those without the disease who test positive. While a particular diagnostic method may not provide a definitive diagnosis of a condition, it suffices if the method provides a positive indication that aids in diagnosis.

“Treatment” is an intervention performed with the intention of preventing the development or altering the pathology or symptoms of a disorder. Accordingly, “treatment” refers to both therapeutic treatment and prophylactic or preventative measures. Those in need of treatment include those already with the disorder as well as those in which the disorder is to be prevented. As used herein, “ameliorated” or “treatment” refers to a symptom which is approaches a normalized value (for example a value obtained in a healthy patient or individual), e.g., is less than 50% different from a normalized value, preferably is less than about 25% different from a normalized value, more preferably, is less than 10% different from a normalized value, and still more preferably, is not significantly different from a normalized value as determined using routine statistical tests.

“Isolating” a small particle refers to the process of removing a particle from a biological sample and separating away other cells which are not particle of the biological sample, e.g. blood. An isolated particle will be generally free from contamination by other cell types, i.e. “homogeneity” or purity” and will generally have the capability of propagation and differentiation to produce mature cells of the tissue from which it was isolated. An isolated particle can exist in the presence of a small fraction of other particle types which do not interfere with the utilization of the particle for analysis or production of other, differentiated cell types. Isolated particles will generally be at least 30%, 40%, 50%, 60%, 70%, 80%, 85%, 90%, 95%, 98%, 99%, or 99.9% pure. Preferably, isolated particles according to the invention will be at least 80% pure.

As used herein, “culturing” refers to propagating or nurturing the particles, by incubating for a period of time in an environment and under conditions, which support particle viability or propagation. Culturing can include one or more of the steps of expanding and enrichment the particles, purifying the particles, according to the invention.

As used herein, the term “autologous particles” is meant to refer to any material derived from the same individual to whom it is later to be re-introduced into the individual.

The term “xenogeneic particles” refers to any particle that derives from a different animal species than the animal species that becomes the recipient animal host in a transplantation or vaccination procedure.

The term “allogeneic particles” refers to any particle that is of the same animal species but genetically different in one or more genetic loci as the animal that becomes the “recipient host”. This usually applies to cells transplanted from one animal to another non-identical animal of the same species.

“Biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils and thymus. Examples of “body fluid samples” include, but are not limited to blood, serum, semen, prostate fluid, seminal fluid, urine, saliva, sputum, mucus, bone marrow, lymph, and tears.

The term “sample” is meant to be interpreted in its broadest sense. A “sample” refers to a biological sample, such as, for example; one or more cells, tissues, or fluids (including, without limitation, plasma, serum, whole blood, cerebrospinal fluid, lymph, tears, urine, saliva, milk, pus, and tissue exudates and secretions) isolated from an individual or from cell culture constituents, as well as samples obtained from, for example, a laboratory procedure. A biological sample may comprise chromosomes isolated from cells (e.g., a spread of metaphase chromosomes), organelles or membranes isolated from cells, whole cells or tissues, nucleic acid such as genomic DNA in solution or bound to a solid support such as for Southern analysis, RNA in solution or bound to a solid support such as for Northern analysis, cDNA in solution or bound to a solid support, oligonucleotides in solution or bound to a solid support, polypeptides or peptides in solution or bound to a solid support, a tissue, a tissue print and the like.

Numerous well known tissue or fluid collection methods can be utilized to collect the biological sample from the subject in order to determine the level of DNA, RNA and/or polypeptide of the variant of interest in the subject. Examples include, but are not limited to, fine needle biopsy, needle biopsy, core needle biopsy and surgical biopsy (e.g., brain biopsy), and lavage. Regardless of the procedure employed, once a biopsy/sample is obtained the level of the variant can be determined and a diagnosis can thus be made.

### Non-Platelet RNA-Containing Particles (NPRCPs) Compositions

To avoid the confusion between particles from platelets, these novel particles embodied in the invention have been termed herein as “non-platelet RNA-containing particles (NPRCPs)”. Although these particles can be isolated using similar centrifugation speed to that of platelets, these NPRCPs are particles that are not produced by megakaryocytes. In addition, electron microscopy shows that these particles do not contain o-granules, cell organelles or glycogen granules as that of the platelets do, indicating that they are not platelets. NPRCPs have a variety of shapes. The NPRCPs were deposited on Dec. 19, 2012 with the American Type Culture Collection (ATCC) under the Budapest Treaty on the International Recognition of the Deposit of Microorganisms for the Purposes of Patent Procedure (ATCC Patent Deposit Designation PTA-13396).

Briefly, the novel particles described herein are a mixed population with different morphology and are very small, contain RNA, but not ribosomal RNA, and are characterized as expressing one or more markers. At least 5 types NPRCP have been identified in this invention. NPRCPs assemble into a group of living organisms that do not have cytoplasmic membrane and nucleus. To avoid the confusion between these living organisms and eukaryotic cells, they have been termed herein as “PFDNCs”. Besides assembling PFDNCs, NPRCPs also assemble into other aggregated products that can further transform into other types of blood-derived cells.

In preferred embodiments isolated NPRCPs comprise at least 5 different types of varieties.

In preferred embodiments isolated NPRCPs comprise small-RNA and micro-RNAs. Small RNAs are less than 200 nt and express more than 100 different micro-RNAs (see Tables).

In preferred embodiments isolated NPRCPs comprise Oct4, DDX4/VASA and sox-2 or their components, or combinations thereof.

In preferred embodiments isolated NPRCPs comprise and express integrin β1, CD45 and CD34 on the surface or combinations thereof.

In preferred embodiments isolated NPRCPs express less than 5% express E-cadherin.

In preferred embodiments an isolated NPRCP produces larger sizes products. One of their products (PFDNCs) has an amoebic-like movement and surrounded by the thin loose membrane. PFDNCs are characterized by their ability to penetrate, cross a eukaryotic cell membrane and get inside of eukaryotic cells, without harming these cells, repeatedly to collect materials.

In preferred embodiments an isolated NPRCP produces other products that further transform into hematopoietic stem cells (HSC) through random combination with themselves and with the blood circulating DNA fragments.

In another preferred embodiment, NPRCPs comprise varying sizes categorized as small, middle and large.

In another preferred embodiment, NPRCPs can be cultured with varying growth and/or differentiation factors. In other embodiments, NPRCPs are cultured with peptides, proteins or nucleotides. These can include hormones, enzymes, cytokines, and the like. In other embodiments, NPRCPs are co-cultured with cells or tissues of a desired type so as to induce nuclear programming of a desired cell type e.g. muscle cells, nerve cells, cardiac cells, kidney cells and the like.

In another preferred embodiment, the NPRCPs are isolated from bone marrow, umbilical cord blood or peripheral blood.

In another preferred embodiment, a composition comprises NPRCPs and their products can be of varying sizes from 0.1 μm to 10 μm. In one embodiment, a composition comprises small size NPRCPs. In another embodiment, a composition comprises middle-sized NPRCPs and their products. In yet another embodiment, a composition comprises large NPRCPs and their products PFDNCs. In yet another embodiment, the composition comprises combinations of two or more sized NPRCPs and their products PFDNCs.

In another preferred embodiment, a composition comprising the subject NPRCPs are characterized in that they contain nuclear granules. In yet another embodiment, a composition comprises NPRCPs and their products PFDNCs, which lack a nucleus and cytoplasmic organelles. This is in contrast to eukaryotic cells.

In another preferred embodiment, a composition comprises the subject NPRCPs and their products PFDNCs are characterized in that they do not have ribosomal RNA. Total RNA isolated from the NPRCPs has short fragments that are less than 200 nt.

The subjects NPRCPs are characterized by their unique morphology taken by electron microscopy. At least 5 variety particles have both nuclear granules and protein membranes are identified.

The NPRCPs are further characterized by their expression of cell surface markers. While it is commonplace in the art to refer to cells as “positive” or “negative” for a particular marker, actual expression levels are a quantitative trait. The number of molecules on the cell surface can vary by several logs, yet still be characterized as “positive”. It is also understood by those of skill in the art that a cell which is negative for staining, i.e. the level of binding of a marker specific reagent is not detectably different from a control, e.g. an isotype matched control; may express minor amounts of the marker. Characterization of the level of staining permits subtle distinctions between cell populations.

The NPRCPs are characterized by their expression of Oct4 in their components. While it is commonplace in the art to refer to cells as “positive” or “negative” for a particular marker, actual expression levels are a quantitative trait. The number of molecules can vary by several logs, yet still be characterized as “positive”. It is also understood by those of skill in the art that a cell which is negative for staining, i.e. the level of binding of a marker specific reagent is not detectably different from a control, e.g. an isotype matched control; may express minor amounts of the marker. Characterization of the level of staining permits subtle distinctions between populations.

The NPRCPs are characterized by their expression of DDX4/VASA in their components. While it is commonplace in the art to refer to cells as “positive” or “negative” for a particular marker, actual expression levels are a quantitative trait. The number of molecules can vary by several logs, yet still be characterized as “positive”. It is also understood by those of skill in the art that a cell which is negative for staining, i.e. the level of binding of a marker specific reagent is not detectably different from a control, e.g. an isotype matched control; may express minor amounts of the marker. Characterization of the level of staining permits subtle distinctions between populations.

The NPRCPs are characterized by their expression of Sox-2 or their components. While it is commonplace in the art to refer to cells as “positive” or “negative” for a particular marker, actual expression levels are a quantitative trait. The number of molecules on the cell surface can vary by several logs, yet still be characterized as “positive”. It is also understood by those of skill in the art that a cell which is negative for staining, i.e. the level of binding of a marker specific reagent is not detectably different from a control, e.g. an isotype matched control; may express minor amounts of the marker. Characterization of the level of staining permits subtle distinctions between populations.

The staining intensity of cells can be monitored by flow cytometry, where lasers detect the quantitative levels of fluorochrome (which is proportional to the amount of cell surface marker bound by specific reagents, e.g. antibodies). Fluorescent activated cell sorting, or FACS, can also be used to separate particle populations based on the intensity of binding to a specific reagent, as well as other parameters such as cell size and light scatter. Although the absolute level of staining may differ with a particular fluorochrome and reagent preparation, the data can be normalized to a control.

In order to normalize the distribution to a control, each particle is recorded as a data point having a particular intensity of staining. These data points may be displayed according to a log scale, where the unit of measure is arbitrary staining intensity. In one example, the brightest stained particles in a sample can be as much as 4 logs more intense than unstained particles. When displayed in this manner, it is clear that the particles falling in the highest log of staining intensity are bright, while those in the lowest intensity are negative. The “low” positively stained cells have a level of staining above the brightness of an isotype matched control, but it is not as intense as the most brightly staining cells normally found in the population. Low positive particles may have unique properties that differ from the negative and brightly stained positive particles of the sample. An alternative control may utilize a substrate having a defined density of marker on its surface, for example a fabricated bead or cell line, which provides the positive control for intensity.

NPRCPs are separated using centrifugation. Blood plasma can be separated by centrifugation the whole blood at about 200×g. Then, the particles in plasma can be isolated by centrifugation at about 6000×g. The particles in the blood proteins can be isolated by removing the red blood cells using RBC lysis buffer, and by removing the nucleated larger cells using centrifugation at about 300×g. Isolated particles can be cultured to remove platelets that have life span less than 10 days and to remove other small cells and dead cell debris.

NPRCPs are separated from a complex mixture of biological materials by techniques that enrich for materials having the characteristics as described. For example, a blood sample may be obtained from a donor or a pool of donors. From this population, NPRCPs may be selected by their expression of Oct4, CD29, CXCR4, c-kit, DDX4/VASA, sox-2, CD45, CD34 or combinations thereof. The cells are optionally selected for one or more of the negative markers recited herein. Further, NPRCPs can be selected for a size ranging from around about 0.1 μm in diameter to about 10 μm in diameter. In addition, they can be selected for their non-cell population. In a preferred embodiment, the NPRCPs comprise varying sizes.

Where the sample is blood cells, no dissociation is required, although removal of red blood cells may be convenient. For example, red blood cells can be removed by RBC lysis buffer, which have variety formula or chemicals, or can be removed by centrifugation. Solid tissues may be dissociated by digestion with a suitable protease, e.g. collagenase, dispase, etc., followed by trituration until dissociated. An appropriate solution is used for dispersion or suspension. Such solution will generally be a balanced salt solution, e.g. normal saline, PBS, Hanks balanced salt solution, etc., supplemented with fetal calf serum or other naturally occurring factors, in conjunction with an acceptable buffer at low concentration, generally from 5-25 mM. Convenient buffers include HEPES, phosphate buffers, lactate buffers, etc.

Separation of NPRCP population will then use affinity separation to provide a substantially pure population. Techniques for affinity separation may include magnetic separation, using antibody-coated magnetic beads, affinity chromatography, cytotoxic agents joined to a monoclonal antibody or used in conjunction with a monoclonal antibody, e.g. complement and cytotoxins, and “panning” with antibody attached to a solid matrix, e.g. plate, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (propidium iodide, 7-AAD). Any technique may be employed which is not unduly detrimental to the viability of the selected cells.

The affinity reagents may be specific receptors or ligands for the cell surface molecules indicated above. The details of the preparation of antibodies and their suitability for use as specific binding members are well known to those skilled in the art. Of particular interest is the use of antibodies as affinity reagents.

The antibodies are added to a suspension of cells, and incubated for a period of time sufficient to bind the available cell surface antigens. The incubation will usually be at least about 5 minutes and usually less than about 60 minutes. It is desirable to have a sufficient concentration of antibodies in the reaction mixture, such that the efficiency of the separation is not limited by lack of antibody. The appropriate concentration is determined by titration. The medium in which the cells are separated will be any medium which maintains the viability of the cells. Examples include, phosphate buffered saline containing from 0.1 to 0.5% serum albumins. Various media are commercially available and may be used according to the nature of the cells, including Dulbecco's Modified Eagle Medium (dMEM), Hank's Basic Salt Solution (HBSS), Dulbecco's phosphate buffered saline (dPBS), RPMI, Iscove's medium, PBS with 5 mM EDTA, etc., frequently supplemented with serum, e.g. BSA, HSA, FCS, etc.

The labeled cells and particles can also be separated by gradient centrifugation by their sizes. The gradients materials can be made, but not limited by Ficoll, Percoll, BSA or sucrose. The centrifugation speed can be different due to the different the materials in the gradient tubes. The centrifugation speed can be from 7000×g down to 200×g.

Various media are commercially available and may be used according to the nature of the cells, including alpha-MEM, dMEM, HBSS, dPBS, RPMI, Iscoves medium, etc., frequently supplemented with serum.

Compositions highly enriched for NPRCPs are achieved in this manner. In preferred embodiments, the subject population will be at or about 50% or more NPRCPs, and usually at or about 80% or more of NPRCPs composition, and may be as much as about 95% or more of the NPRCPs population. The enriched NPRCPs population may be used immediately, or may be frozen at liquid nitrogen temperatures and stored for long periods of time, being thawed and capable of being reused. Once thawed, the NPRCPs may be expanded in culture for proliferation and differentiation.

The compositions thus obtained have a variety of uses in clinical therapy, research, development, and commercial purposes. For therapeutic purposes, for example, NPRCPs and their products PFDNCs may be administered to enhance tissue maintenance or repair of muscle for any perceived need, such as an inborn error in metabolic function, the effect of a disease condition, or the result of significant trauma.

In preferred embodiments, NPRCPs and their products PFDNCs are used in the prevention or treatment of various diseases or disorders, in particular, those associated with degeneration of cells or tissues. In other embodiments, NPRCPs or their products PFDNCs are used in the treatment of wounds, burns, broken or cracked bones and the like.

In a preferred embodiment, a method of regenerating cells or tissues in vivo comprises administering to a patient in vivo, an effective amount of NPRCPs and/or their products PFDNCs; and, regenerating the cells or tissues. NPRCPs and/or their products PFDNCs can be selected based on tissue to be generated. In other embodiments, combinations of NPRCPs and/or their products PFDNCs are administered to the patient. In other embodiments, NPRCPs and/or their products PFDNCs can be administered based on the expression of a particular surface marker or the expression profile of a particular marker or combination of markers. For example, the data herein show that less than 30% NPRCPs, mostly the larger size NPRCPs or PFDNCs, express c-kit. About 80% of NPRCPs and PFDNCs (large and small) express integrin 131, Oct4, sox2 and actin, however the expression on large NPRCPs is much stronger than that of on small ones. In addition, it was found that about 40% of NPRCPs, mainly the middle sizes, express DDX4/VASA. The expression is strong and in a patched pattern in NPRCPs. The administered PFDNCs undergo nuclear programming during maturation and can be pre-cultured with growth and/or differentiation factors or can be administered to the patient allowing for the maturation of the PFDNCs in the desired in vivo locale and thus, are subjected to the micro environment of the surrounding cells and tissues allowing for the nuclear programming of the pre-cells. Thus in preferred embodiments, the pre-cells are optionally cultured and expanded ex vivo prior to administering to a patient. In other embodiments, the pre-cells are optionally cultured with desired differentiation and/or growth factors ex vivo, prior to administering to a patient. In other preferred embodiments, NPRCPs are pluripotent and differentiate into multiple cell lineages. NPRCPs have the ability to give rise to types of cells that develop from the three germ layers (mesoderm, endoderm, and ectoderm) from which all the cells of the body arise.

In yet other preferred embodiments, NPRCPs are obtained or isolated from any source. For example, NPRCPs are isolated from sources comprising: autologous, heterologous, syngeneic, allogeneic or xenogeneic sources. The methods of selection can include further enrichment or purification procedures or steps for NPRCPs isolation by selection for specific markers, NPRCPs size, etc. NPRCPs may be obtained from any mammalian species, e.g. human, equine, bovine, porcine, canine, feline, rodent, e.g. mice, rats, hamster, primate, etc.

In other embodiments, NPRCPs and/or their products PFDNCs are administered to patients in the treatment of wounds, including but not limited to trauma, surgical and infected wounds; surface ulcers including but not limited to chronic ulcers, diabetic ulcers, decubital ulcer, and lower limb vascular disease, and other non-healing wounds as result of poor blood flow; wounds and/or erosions caused by bacterial and viral infection, such as vaginitis, cervical erosion, gingivitis; wounds due to dilation and enlargement of veins such as hemorrhoids; herpes simplex corneal ulcer, subcutaneous tissue ulcer, radiation-caused skin ulcer, wounds caused by wind and cold such as chilblain and chapped skin.

In other embodiments, NPRCPs and/or their products PFDNCs may be used to regenerate all type of skin cells by physiologically repair damaged tissue(s) of the skin without scars, such as the skin of a deep second degree burn (or partial thickness burn) that has destroyed the epidermis, the basal layer, and severely damaged the dermis. The methodology may also be used to regenerate skin with restoration of structures and functions of the epidermis, dermis and various appendages of the skin. For example, a patient with both epidermis and dermis destroyed by fire or chemical, i.e., superficial third degree burn or full thickness burn, can be treated with the methodology without substantial loss of physiological functions of the skin, including those of the appendages.

In other embodiments, NPRCPs and/or their products PFDNCs are useful in transplantation, including the regeneration of skin, e.g. in the treatment of burns, surgery; following traumatic damage, for cosmetic purposes, in the treatment of keloids and fibrosis; and the like. For such purposes the NPRCPs may be introduced systemically, e.g. by i.v. injection.

In another embodiment, NPRCPs and/or their products PFDNCs may be used to regenerate cardiomyocytes to physiologically repair damaged tissue(s) of the heart damage, for example from acute or chronic heart disease.

In some preferred embodiments, NPRCPs and/or their products PFDNCs can be used to regenerate hepatocytes by transplantation to liver cirrhosis patients.

In some preferred embodiments, NPRCPs and/or their products PFDNCs can be used to regenerate kidney endothelial cells, renal ducts and interstitial cells by transplantation to acute kidney damaged patients.

In some preferred embodiments, NPRCPs and/or their products PFDNCs can be used to regenerate neurons by transplantation to brain damaged or stroke patients.

In some preferred embodiments, NPRCPs and/or their products PFDNCs can be used to regenerate brain cells by transplantation to Alzheimer's disease (AD) patients.

In some preferred embodiments, NPRCPs and/or their products PFDNCs can be used to regenerate bone or cartilage by transplantation to bone or knee damaged patients.

In some preferred embodiments, NPRCPs and/or their products PFDNCs can be used to regenerate gastric and intestinal cells by transplantation to stomach or gut damaged patients.

In some preferred embodiments, NPRCPs and/or their products PFDNCs can be used for regenerate skeletal muscle by transplantation to muscle atrophy patients.

Improvement in an individual receiving the therapeutic compositions provided herein can also be assessed by subjective metrics, e.g., the individual's self-assessment about his or her state of health following administration.

In certain embodiments, the methods of treatment provided herein comprise inducing the therapeutic NPRCPs and/or PFDNCs to differentiate into the different lineages depending on the desired outcome.

Administration of NPRCPs and/or PFDNCs, or therapeutic compositions comprising such particles, to an individual in need thereof, can be accomplished, e.g., by transplantation, implantation (e.g., of the particles themselves or the particles as part of a matrix-cell combination), injection (e.g., directly to the site of the disease or condition, for example, directly to an ischemic site in the heart of an individual who has had a myocardial infarction), infusion, delivery via catheter, or any other means known in the art for providing therapy.

In one embodiment, the therapeutic compositions are provided to an individual in need thereof, for example, by injection into one or more sites in the individual. In other preferred embodiments, the NPRCPs and/or PFDNCs can home to the diseased or injured area.

Also provided herein are kits for use in the treatment of patients in need thereof. The kits provide the therapeutic NPRCPs and/or PFDNCs composition which can be prepared in a pharmaceutically acceptable form, for example by mixing with a pharmaceutically acceptable carrier, and an applicator, along with instructions for use. Ideally the kit can be used in the field, for example in a physician's office, or by an emergency care provider to be applied to a patient.

In some aspects of the methods of treatment provided herein, the NPRCPs and/or PFDNCs are administered with tissue specific stem cells.

In some embodiments, populations of NPRCPs and/or PFDNCs are incubated or are administered to a patient in the presence of one or more factors which stimulate stem or progenitor cell differentiation along a desired pathway. Such factors are known in the art; determination of suitable conditions for differentiation can be accomplished with routine experimentation. Such factors include, but are not limited to factors, such as growth factors, chemokines, cytokines, cellular products, demethylating agents, and other stimuli which are now known or later determined to stimulate differentiation. For example, inclusion of demethylation agents tends to allow the cells to differentiate along mesenchymal lines, toward a cardiomyogenic pathway. Differentiation can be determined by, for example, expression of at least one of cardiomyosin, skeletal myosin, or GATA4; or by the acquisition of a beating rhythm, spontaneous or otherwise induced; or by the ability to integrate at least partially into a patient's cardiac muscle without inducing arrhythmias. Demethylation agents that can be used to initiate such differentiation include, but are not limited to, 5-azacytidine, 5-aza-2′-deoxycytidine, dimethylsulfoxide, chelerythrine chloride, retinoic acid or salts thereof, 2-amino-4-(ethylthio)butyric acid, procainamide, and procaine.

NPRCPs and/or PFDNCs, can be provided therapeutically or prophylactically to an individual, e.g., an individual having a disease, disorder or condition of, or affecting, the brain, kidney, muscles, liver, heart, or skeletal system.

The NPRCPs and/or PFDNCs may be administered to an individual in the form of a therapeutic composition comprising the particles and another therapeutic agent, such as insulin-like growth factor (IGF), platelet-derived growth factor (PDGF), epidermal growth factor (EGF), fibroblast growth factor (FGF), vascular endothelial growth factor (VEGF), hepatocyte growth factor (HGF), IL-8, an antithrombogenic agent (e.g., heparin, heparin derivatives, urokinase, and PPack (dextrophenylalanine proline arginine chloromethylketone); antithrombin compounds, platelet receptor antagonists, anti-thrombin antibodies, anti-platelet receptor antibodies, aspirin, dipyridamole, protamine, hirudin, prostaglandin inhibitors, and/or platelet inhibitors), an antiapoptotic agent (e.g., EPO, EPO derivatives and analogs, and their salts, TPO, IGF-I, IGF-II, hepatocyte growth factor (HGF), or caspase inhibitors), an anti-inflammatory agent (e.g., P38 MAP kinase inhibitors, statins, IL-6 and IL-1 inhibitors, Pemirolast, Tranilast, Remicade, Sirolimus, nonsteroidal anti-inflammatory compounds, for example, acetylsalicylic acid, ibuprofen, Tepoxalin, Tolmetin, or Suprofen), an immunosuppressive or immunomodulatory agent (e.g., calcineurin inhibitors, for example cyclosporine, Tacrolimus, mTOR inhibitors such as Sirolimus or Everolimus; anti-proliferatives such as azathioprine and mycophenolate mofetil; corticosteroids, e.g., prednisolone or hydrocortisone; antibodies such as monoclonal anti-IL-2Rα receptor antibodies, Basiliximab, Daclizuma, polyclonal anti-T-cell antibodies such as anti-thymocyte globulin (ATG), anti-lymphocyte globulin (ALG), and the monoclonal anti-T cell antibody OKT3, and/or an antioxidant (e.g., probucol; vitamins A, C, and E, coenzyme Q-10, glutathione, L cysteine, N-acetylcysteine, or antioxidant derivative, analogs or salts of the foregoing). In certain embodiments, therapeutic compositions comprising the NPRCPs and/or PFDNCs optionally comprise one or more additional cell types, e.g., adult cells (for example, fibroblasts or endodermal cells), or stem or progenitor cells. Such therapeutic agents and/or one or more additional cells can be administered to an individual in need thereof individually or in combinations or two or more such compounds or agents.

In certain embodiments, the individual to be treated is a mammal. In a specific embodiment the individual to be treated is a human. In specific embodiments, the individual is a livestock animal or a domestic animal. In other specific embodiments, the individual to be treated is a horse, sheep, cow or steer, pig, dog or cat.

In another preferred embodiment, the population of NPRCPs and/or PFDNCs is at least about 80% pure as compared to a control sample isolated from a patient, preferably, the NPRCPs and/or PFDNC population is about 90% pure as compared to a control sample, preferably, the NPRCPs and/or PFDNC population is about 95%, 96%, 97%, 98%, 99%, 99.9% pure as compared to a control sample.

In another preferred embodiment, the NPRCPs and/or PFDNC populations can be used in any assay desired by the end user, such as for example, expressing a non-native or foreign molecule, a native molecule which may or may not be activated in the particles. Examples of such molecules can be growth factors, receptors, ligands, therapeutic agents, etc. The molecules can be selected by the end user for expression by the isolated NPRCPs and/or PFDNCs depending on the end user's need. The molecules comprise, for example, a polypeptide, a peptide, an oligonucleotide, a polynucleotide, an organic or inorganic molecule.

In another embodiment, the NPRCPs and/or PFDNC can be transformed with an expression vector encoding for a desired molecule, e.g. cytokine, protein, enzyme etc.

The term “expression vector” as used herein refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules, siRNA, ribozymes, and the like. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well.

By “encoding” or “encoded”, “encodes”, with respect to a specified nucleic acid, is meant comprising the information for translation into the specified protein. A nucleic acid encoding a protein may comprise non-translated sequences (e.g., introns) within translated regions of the nucleic acid, or may lack such intervening non-translated sequences (e.g., as in cDNA). The information by which a protein is encoded is specified by the use of codons. Typically, the amino acid sequence is encoded by the nucleic acid using the “universal” genetic code.

As used herein, “heterologous” in reference to a nucleic acid is a nucleic acid that originates from a foreign species, or, if from the same species, is substantially modified from its native form in composition and/or genomic locus by deliberate human intervention. For example, a promoter operably linked to a heterologous structural gene is from a species different from that from which the structural gene was derived, or, if from the same species, one or both are substantially modified from their original form. A heterologous protein may originate from a foreign species or, if from the same species, is substantially modified from its original form by deliberate human intervention.

A wide variety of host/expression vector combinations may be employed in expressing the DNA sequences of this invention. Useful expression vectors, for example, may consist of segments of chromosomal, non-chromosomal and synthetic DNA sequences. Suitable vectors include derivatives of SV40 and known bacterial plasmids, e.g., E. coli plasmids col E1, pCR1, pBR322, pMal-C2, pET, pGEX (Smith et al., Gene 67:31-40, 1988), pMB9 and their derivatives, plasmids such as RP4; phage DNAs, e.g., the numerous derivatives of phage 1, e.g., NM989, and other phage DNA, e.g., M13 and filamentous single stranded phage DNA; yeast plasmids such as the 2μ plasmid or derivatives thereof, vectors useful in eukaryotic cells, such as vectors useful in insect or mammalian cells; vectors derived from combinations of plasmids and phage DNAs, such as plasmids that have been modified to employ phage DNA or other expression control sequences; and the like.

Yeast expression systems can also be used according to the invention to express STING. For example, the non-fusion pYES2 vector (XbaI, SphI, ShoI, NotI, GstXI, EcoRI, BstXI, BamH1, SacI, Kpn1, and HindIII cloning sites; Invitrogen) or the fusion pYESHisA, B, C (Xba1, SphI, ShoI, NotI, BstXI, EcoRI, BamH1, SacI, KpnI, and HindIII cloning sites, N-terminal peptide purified with ProBond resin and cleaved with enterokinase; Invitrogen), to mention just two, can be employed according to the invention. A yeast two-hybrid expression system can be prepared in accordance with the invention.

One preferred delivery system is a recombinant viral vector that incorporates one or more of the polynucleotides therein, preferably about one polynucleotide. Preferably, the viral vector used in the invention methods has a pfu (plague forming units) of from about 10⁸ to about 5×10¹⁰ pfu. In embodiments in which the polynucleotide is to be administered with a non-viral vector, use of between from about 0.1 nanograms to about 4000 micrograms will often be useful e.g., about 1 nanogram to about 100 micrograms.

The invention also comprehends methods for preparing compositions, such as pharmaceutical compositions, including NPRCPs and/or PFDNCs and/or at least one cytokine, for instance, for use in inventive methods for treating cardiovascular disease, heart failure or other cardiac conditions. In one embodiment, the pharmaceutical composition comprises isolated NPRCPs and/or PFDNCs and a pharmaceutically acceptable carrier. In a preferred aspect, the methods and/or compositions, including pharmaceutical compositions, comprise effective amounts of NPRCPs and/or PFDNCs.

In an additionally preferred aspect, the pharmaceutical compositions of the present invention are delivered via injection. These routes for administration (delivery) include, but are not limited to, subcutaneous or parasternal including intravenous, intra-arterial (e.g. intracoronary), intramuscular, intraperitoneal, intramyocardial, transendocardial, trans-pericardial, intranasal administration as well as intra-articular, intra-thecae, and infusion techniques. Accordingly, the pharmaceutical composition is preferably in a form that is suitable for injection. When administering a therapeutic of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion, and by the use of surfactants. Nonaqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds. Sterile injectable solutions can be prepared by incorporating the compounds utilized in practicing the present invention in the required amount of the appropriate solvent with various amounts of the other ingredients, as desired.

The pharmaceutical compositions of the present invention, e.g., comprising a therapeutic dose of NPRCPs and/or PFDNCs, can be administered to the subject in an injectable formulation containing any compatible carrier, such as various vehicles, adjuvants, additives, and diluents; or the compounds utilized in the present invention can be administered parenterally to the subject in the form of slow-release subcutaneous implants or targeted delivery systems such as monoclonal antibodies, iontophoretic, polymer matrices, liposomes, and microspheres.

In one embodiment, a composition of the present invention can be administered initially, and thereafter maintained by further administration. For instance, a composition of the invention can be administered in one type of composition and thereafter further administered in a different or the same type of composition. For example, a composition of the invention can be administered by intravenous injection to bring blood levels to a suitable level. The subject's levels are then maintained by an oral dosage form, although other forms of administration, dependent upon the subject's condition, can be used. The quantity of the pharmaceutical composition to be administered will vary for the subject being treated.

The precise determination of what would be considered an effective dose may be based on factors individual to each subject, including their size, age, area of damaged myocardium, and amount of time since damage. Thus, the skilled artisan can readily determine the dosages and the amount of compound and optional additives, vehicles, and/or carrier in compositions to be administered in methods of the invention. Typically, any additives (in addition to the active stem cell(s) and/or cytokine(s)) are present in an amount of 0.001 to 50 wt % solution in phosphate buffered saline, and the active ingredient is present in the order of micrograms to milligrams, such as about 0.0001 to about 5 wt %, preferably about 0.0001 to about 1 wt %, most preferably about 0.0001 to about 0.05 wt % or about 0.001 to about 20 wt %, preferably about 0.01 to about 10 wt %, and most preferably about 0.05 to about 5 wt %. Of course, for any composition to be administered to an animal or human, and for any particular method of administration, it is preferred to determine therefore: toxicity, such as by determining the lethal dose (LD) and LD₅₀ in a suitable animal model e.g., rodent such as mouse; and, the dosage of the composition(s), concentration of components therein and timing of administering the composition(s), which elicit a suitable response. Such determinations do not require undue experimentation from the knowledge of the skilled artisan, this disclosure and the documents cited herein. The time for sequential administrations can be ascertained without undue experimentation. Examples of compositions comprising a therapeutic of the invention include liquid preparations for orifice, e.g., oral, nasal, anal, vaginal, peroral, intragastric, mucosal (e.g., perlingual, alveolar, gingival, olfactory or respiratory mucosa) etc., administration such as suspensions, syrups or elixirs; and, preparations for parenteral, subcutaneous, intradermal, intramuscular or intravenous administration (e.g., injectable administration), such as sterile suspensions or emulsions. Such compositions may be in admixture with a suitable carrier, diluent, or excipient such as sterile water, physiological saline, glucose or the like. The compositions can also be lyophilized. The compositions can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Pharmaceutical compositions of the invention are conveniently provided as liquid preparations, e.g., isotonic aqueous solutions, suspensions, emulsions or viscous compositions which may be buffered to a selected pH.

The invention has been described in detail with reference to preferred embodiments thereof. However, it will be appreciated that those skilled in the art, upon consideration of this disclosure, may make modifications and improvements within the spirit and scope of the invention. The following non-limiting examples are illustrative of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

While various embodiments of the present invention have been described above, it should be understood that they have been presented by way of example only, and not limitation. Numerous changes to the disclosed embodiments can be made in accordance with the disclosure herein without departing from the spirit or scope of the invention. Thus, the breadth and scope of the present invention should not be limited by any of the above described embodiments.

### Materials and Methods

Kidney Regeneration (Ischemic Damage):

Eight to ten week old female Balb/c mice were anesthetized. After the fur was shaved, the abdominal skin was cleaned and opened.

Kidney arteries on each side were ligated at the same time with use of a 4-0 suture. After 45 min, sutures were removed to reopen the ligated arteries. After the abdomen skin was closed, 20 million GFP-mouse blood derived NPRCPs in 150 μl normal saline was transplanted by tail-vein injection with use of a 26-gauge needle. The control groups underwent tail-vein injection of saline. Mice were sacrificed at day 1 and weeks 1, 2, 3, 4, and 6 after transplantation. Kidneys were removed and fixed for histology.

Neuron Regeneration (Ischemic Damage):

Focal cerebral ischemia was induced by middle cerebral artery occlusion (MCAO). Briefly, after anesthetization with chloral hydrate (400 mg/kg, i.p.), the ipsilateral external carotid artery (ECA) was ligated. A 6-0 nylon monofilament suture, blunted at the tip and coated with 1% poly-L-Lysine, was inserted through the right common carotid artery (CCA) inot the internal carotid artery (ICA) and advanced approximately 10 mm distal to the ECA/ICA bifurcation to occlude the origin of the middle cerebral artery (MCA) at the junction of the Circle of Willis. The suture was withdrawn 90 min after occlusion to allow reperfusion. Sham-operated mice underwent identical surgery, except that the intraluminal filament was not inserted. After surgery, mice were kept for about 2 hours in a warm box heated by lamps to maintain body temperature. 20 million NPRCPs isolated from GFP-transgenic mice were transplanted via tail-vein to each mouse within 5 hours after brain surgery.

Skin Regeneration (Acute Wounds):

Two wounds were created by a 6 mm biopsy punch on the dorsal skin of each Balb/c mouse. 20 million NPRCPs, collected from GFP-transgenic mice and cultured for three weeks, were transplanted within one hour after wound creation. Wounds were collected on 2, 4, 6, 8 and 10 days after transplantation.

Smooth Muscle Regeneration (Acute Wounds):

Two wounds were created by a 6 mm biopsy punch on the dorsal skin of each Balb/c mouse. 20 million NPRCPs, collected from GFP-transgenic mice and cultured for three weeks, were transplanted within one hour after wound creation. Wounds were collected on 2, 4, 6, 8 and 10 days after transplantation.

Cardiomyocyte Regeneration (by Ischemic Damage):

Left anterior descending (LAD) coronary artery ligation and re-perfusion. Ten-to twelve weeks old male Balb/c were used for the LAD surgery. Each mouse was anesthetized by inhaling 3% of isoflurane. The anesthetization was kept by intra-tracheal tubing ventilated with a mouse ventilator set at 1.5% of isoflurane. Using dissecting microscopy, the chest was opened along the parasternal left side. The pericardium was then gently dissected to visualize the heart and the left coronary artery. The LAD coronary artery was blocked at a medium level by ligation with a 9-0 nylon suture, and reperfused by removing the ligature 30 minutes later. The chest wall was closed with interrupted stitches using 6-0 polyglactin suture. Then, the skin was closed with a 6-0 silk suture. 20 million NPRCPs, collected from GFP-transgenic mice and cultured for 3 weeks, were transplanted into each mouse via tail-vein injection. Hearts were collected at 2, 4 and 8 weeks after the transplantation.

Liver Regeneration (Toxic Damage):

12 week old C57BL6 mice were intraperitoneally administered Streptozotocin (STZ) 100 mg/kg at every other day for three times (Monday, Wednesday and Friday). Three days later, the blood sugar was measured. Mice with a blood sugar higher than 13 mmol/L was used for NPRCP transplantation. Livers were collected at 2, 4, 7 and 9 days after transplantation.

Pancreatic Regeneration (Toxic Damages):

Twelve week old C57BL6 mice were intraperitoneally administered Streptozotocin 100 mg/kg every other day for three times (Monday, Wednesday and Friday). Three days later the blood sugar was measured. Mice that had blood sugar higher than 13 mmol/L were considered successful for diabetic modeling. 20 million NPRCP, collected from GFP-transgenic mice and cultured for 3 weeks, were transplanted into each diabetic mouse via tail-vein injection. Pancreases were collected on 2, 4, 7 and 9 days after transplantation. For the two month diabetic studies, each mouse received the second NPRCP transplantation 10 days after the first. Pancreases were collected 2 months after the first transplantation.

### Example 1

### Identification of NPRCPs and PFDNCs

Human umbilical cord blood was collected after the delivery of healthy newborns. Plasma was removed by centrifugation at 200×g for 10 min. The plasma portion was centrifuged again at 5000×g for 10 min. The cellular portion was transferred into red blood cell lysis buffer (155 mM ammonium chloride, 10 mM potassium hydrogen carbonate, and 0.1 mM EDTA) for 20 to 30 min. Then, the lysate was centrifuged at 300×g for 10 min. The upper portion was then centrifuged at 5000×g for 10 min. After aspirate the supernatants and the fluffy cell membrane layer, the pellets were resuspended in PBS containing 0.1 mM EDTA and centrifuged again at 5000×g for 10 min.

The pellets in both plasma and cellular portions were either pooled together or separated cultured on plates using alpha-MEM with 20% fetal bovine serum. Medium was changed every other day and NPRCP growth was observed. The trace of red blood cells and platelet disappear after 10 days in culture.

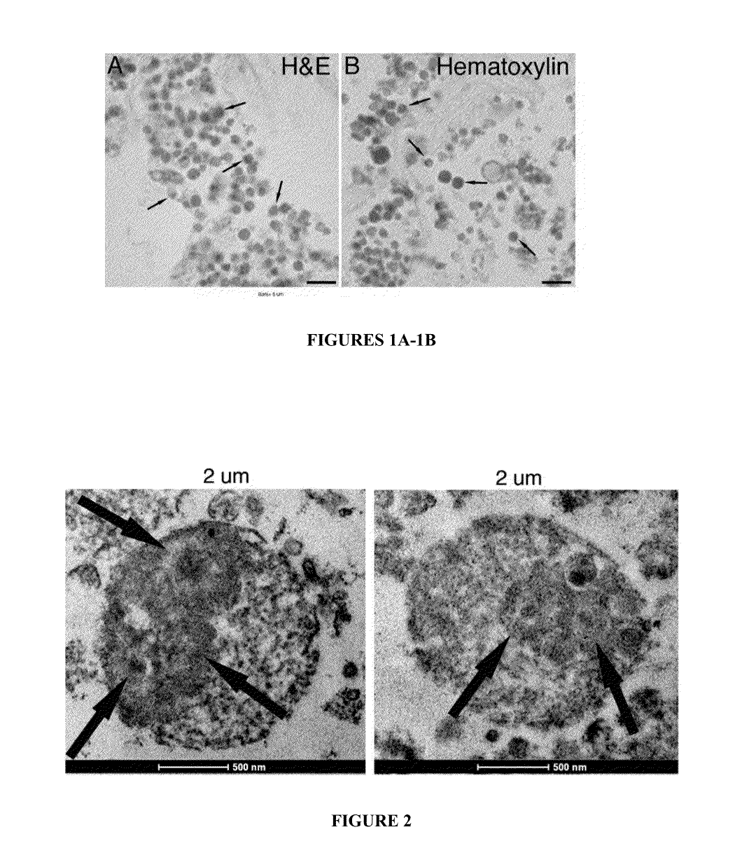

Identification and Characterization of NPRCPs (FIG. 1-12).

NPRCPs contain both proteins and RNA. FIGS. 1A and 1B show that cultural enriched NPRCPs stained with hematoxylin and eosin (H&E) and hematoxylin. All NPRCPs are smaller than 5 μm. Hematoxylin stains for nuclear materials and eosin stains mostly for proteins. Small NPRCPs stain positive for H&E, with more hematoxylin stains at one side of the membrane (arrows) and eosin in the rest areas.

NPRCPs are a mixed population. To discern the detailed structure of these NPRCPs, electron microscopy was used for examination. EM images revealed more than 5 types of different particles. They were listed as 1) the core-like granules type (FIG. 2). These type particles contain large nuclear granules (arrows). These nuclear granules have dense core-like structure and vary in sizes (0.1-0.5 μm). This type NPRCPs' sizes arrange from 2 μm to 5 μm.

2) The loose membrane type (FIG. 3). This type of NPRCPs has extremely thin membrane outside and contains scattered dense nuclear granules inside the membrane. They also have a layered structure inside. Due to the flexibility of membrane structures, their shape can be very different.

3) The solid particle type (FIG. 4). This type of NPRCPs does not have loose membrane. They contain small granules, dense nuclear materials and less dense areas. They are round shapes. Most nuclear granules locate at one side of the particles (see the 4-5 μm) or beneath the membrane.

4) The condensed material type (FIG. 5). This type of NPRCPs contains dense materials that distribute evenly in the components. Some nuclear granules can be seen beneath the membrane. In addition, their membrane is thick and has irregular protrusions.

5) The uniform and not condensed type (FIG. 6). This type of NPRCPs contains small nuclear granules and fiber-like structure that distribute evenly inside the components. These NPRCPs are in round shape with thin membranes.

FIG. 7 shows that the NPRCPs contain only less than 200 nt RNA fragments. RNA from NPRCPs and pre-cells were collected and run on an Agilent 2100 Bio-analyzer. The RNA bands were analyzed and the results indicate that NPRCPs have only RNAs that are less than 200 nt. Ribosomal RNAs can be seen in the pre-cell samples. The data was repeatable in three separated samples.

FIGS. 8A-8D show the specific marker expression in the components of the NPRCPs. Oct4 and Sox-2, the embryonic stem cell markers are expressed on about 80% NPRCPs. In comparison, DDX4/VASA, the germ cell marker is expressed on about 60% of NPRCPs. Oct4, sox2 and DDX4/VASA are expressed as particle shapes on NPRCPs, suggesting that their expression is specific. About 40% of them co-express tubulin and all of them express actin. NPRCPs do not stain to DAPI, suggesting that they do not have nucleus and the nuclear components inside are weak. Scale bars=10 μm.

FIG. 9 shows immunofluorescent stain of the surface markers on NPRCPs. NPRCPs stain positive to c-kit (about 30%), integrin β1 (more than 80%) and E-cadherin (less than 5%). They do not express CD90. Scale bars=10 μm.

FIG. 10 shows immunofluorescent study of CD45 and CD34 expression. It was have found that CD45, the blood stem cell marker is expressed on most small round-shaped particles. In the lower panel, a group of DNA stains to DAPI, although has not become the nucleus, and strongly to CD34, indicating the CD34 expressing group is a pre-cell. Scale bars=10 μm.

FIG. 11 shows cultured human umbilical cord blood-derived NPRCPs were imaged by a 40× lens using a Nikon microscopy. Images show that NPRCPs increase their sizes by fusion with multiple small particles. Scale bars=10 μm.

FIG. 12 shows the evidence of in vitro expansion of NPRCPs. Images show that NPRCPs can be enriched during culture. Images were under the same field at day 0 (left) and 5 days later (right). The number of NPRCPs is significantly increased, evidencing that NPP can be enriched in the medium.

Evidence that NPRCPs are Released by Oct4-Expressing Pre-Stem Cells (FIG. 13-18).

FIG. 13 shows that NPRCPs are produced from non-nucleated cells. NPRCPs in human umbilical cord blood cells were isolated and dropped on glasses and stained to haematoxylin and eosin. Numerous NPRCPs having spindle shapes (left) and round shapes (right) were identified. Both types NPRCPs locate closely to a larger cellular structure that do not have nucleus (arrows).

FIG. 14 shows that three different types of NPRCPs are identified. The spindle shape (left) and round shape (middle) NPRCPs were identified in human umbilical cord blood. The short-rod shaped NPRCPs (right) were identified in vivo in mouse tissues after transplantation of mouse NPRCPs. All three types NPRCPs were stained to haematoxylin and eosin. No specific eosin stains on all of them, indicating these NPRCPs contain more nuclear materials than proteins. Scale bars=10 μm.

FIG. 15 shows that the NPRCP-producing cells are nucleated after differentiation. NPRCPs were collected from human umbilical cord blood and dropped on cover glass and stain with H&E. A dense haematoxylin stained nucleus (arrow on left) is seen on the left image compare to the non-nucleated cell (arrow on right). These data indicate that these NPRCP-producing cells can be nucleated when they are differentiated. Scale bars=10 μm.

FIG. 16 shows the live images of NPRCP-producing cells. Images were taken in cultured human umbilical cord blood. Numerous small NPRCPs (left) locate around a small cellular structure. Relative larger sized NPRCPs locate near a small cellular structure (right). These data suggest that these cellular structures produce small NPRCPs that further grow extracellularly. Scale bars=10 μm.

FIG. 17 shows that NPRCP-producing cells become nucleated after differentiation. Human umbilical cord blood cells were cultured. Image shows a nucleated cell that is releasing numerous small particles. This cell attaches to the bottom of cultural plate and has clear nucleus. This data indicate that these NPRCP-producing small cellular structures in FIG. 15 can be differentiated, which is characterized by appearing nucleus.

FIG. 18 shows that NPRCP-producing cellular structures express Oct4. Human umbilical cord blood-derived cells were dropped on cover glass and stained to Oct4. The upped panel shows a cellular structure containing numerous small DAPI-dense particles. A small particle is budding off this cellular structure (arrows). The lower four images show a strong oct4-expressing structure that has weak DAPI stains. It shows weak DAPI stain only when light is over exposed. Numerous small oct4- and DAPI-positive particles locate around this structure. These images indicate that NARCP-producing cellular structures do not have nucleus at early stages. Scale bars=10 μm.

PFDNC Formation and Characterization, Including their Morphologies and Surface Markers (FIG. 19-24).

FIG. 19 shows that NPRCPs fuse into non-nucleated cells. Time-lapse images show a group of NPRCPs that fuse into a cellular structure in 11 min. Numerous small vesicles surround the structure. Scale bar=10 μm.

FIG. 20 shows that non-nucleated PFDNCs are developed from at least 2 NPRCPs having the size about 3-10 μm. One particle has the thin membrane can be seen when they reach the maturation (arrow). Scale bars=10 μm.

FIG. 21A to 21F show the different morphologies of PFDNCs when they are in the cultural plates. PFDNCs are characterized by having amoebic-like motion. They twist their bodies in the cultural medium. Images in G-I show the differentiated PFDNCs. They have the sizes from 10 to 20 μm. They change their motion as crawling on the plates. Scale bars=10 μm.

FIG. 22 shows the expression and morphology of active integrating DNA and RNA (PFDNCs). Cropped images of inactive (A) and active (B) PFDNCs. H&E (C) and tubulin immunofluorescent staining (D) of PFDNCs for SOX2, integrin β1, tubulin and DDX4. The middle 2 panels show immunofluorescent staining of PFDNCs. All merged images were stained with DAPI (blue). Images were taken by upright conventional fluorescent microscopy. Scale bars=10 μm.

FIGS. 23A-23B show that PFDNCs are derived from collection of cytoplasmic material by NPRCPs. Electron microscopy images of 2 pre-cellular structures, each about 5 μm (A) and 8 μm (B) with an NPRCP in the center (arrows), which suggests that the core of PFDNCs are derived from NPRCPs.

FIGS. 24A-24C show the FACS results of PFDNCs. FIGS. 24A, 24B and 24C are the results of the detection of surface markers on PFDNCs by fluorescence activated cell sorter (FACS). FIG. 24A=control group; FIG. 24B=CD29 and FIG. 24C=CXCR4 expression. FACS analyses demonstrate that PFDNCs express more that 60% PFDNC express CD29. About 40% of them express CXCR4. They do not express E-cadherin. The figures in the following are the results from FACS experiment. Results were repeatable after performing other experiments.

Evidence of Single Nucleate Cell Formation by PFDNC's Direct Transdifferentiation (FIG. 25-30).

FIG. 25 shows that NPRCPs and PFDNCs that become nucleated cells. This figure describes the transformation of NPRCPs (upper left image) that are about 1 to 2 μm to the larger sized NPRCPs (upper right image). NPRCPs further fuse with proteins (arrows in lower left image) and become differentiated. The differentiated PFDNCs still do not have typical nucleus comparing to the nucleated cells (lower right image). Scale bars=10 μm.

FIG. 26 shows that newly PFDNC-derived cells are stem cells. H&E stains (A-D) indicate that PFDNCs do not have strong eosin stains as that of nucleated cells. During PFDNC's differentiation, their protein contents increase while the nucleus is still not formed yet. Immunofluorescent stains indicate that PFDNCs and differentiated PFDNCs stain to Oct4 (Arrow in H), however the fully nucleated cells have no oct4 stains (arrows in lower panel). Scale bars=10 μm.

FIG. 27 shows images of mature PFDNCs that get inside of the cells to take materials from these cells. two mature PFDNCs (arrows) are inside the eukaryotic cells at 28 min (thin arrow) and 55 min (thick arrow). They become differentiated PFDNCs after. Scale bar=50 μm.

FIG. 28 shows images show that differentiated PFDNCs can transform into eukaryotic cells by fusion to each other and get inside of the cytoplasmic membrane. This process can occur in a few min. The upper panel shows fusion of two pairs differentiated PFDNCs become two pre-cells. The lower panel shows one pre-cell fuse into a cytoplasmic membrane and become a cell in 9 min. Scale bars=10 μm.

FIG. 29 shows fusion-differentiation of cells by PFDNCs. Time-lapse images (A) show fusion of PFDNCs. Three PFDNCs (arrows) fuse to become a cell within 18 min. Bar=10 μm.

FIG. 30 shows the PFDNC-derived pre-cells to that has DNA (dense blue in the large one) in the nuclear areas. The dense blue stain in the large pre-cell is not round, suggesting this pre-cell is forming the nucleus. Thin and loose membrane can be seen in all three images, suggesting that they are from the same type particles. Scale bars=5 μm.

Evidence of Multiple Nucleated Cell Formation by PFDNC's Fusion-Differentiation (FIG. 31A, 31B-34).

FIGS. 31A, 31B show NPRCP fusion-differentiation. Co-cultured NPRCPs were examined by microscopy and video recorded. The time-lapse images were cropped from snapshots of a video record to show a group of small particles fusing into a cellular structure in 150 min (A). Staining for OCT4 expression in co-cultured cells (B). Scale bar in A=20 μm; B=10 μm.

FIG. 32 shows NPRCP fusion-derived large cells release small non-nucleated cells. A live cell (A) in the culture plate protrudes at least 2 cellular portions (arrows in A). H&E staining of a large cell (B) shows irregular haematoxylin staining. The protruding cytoplasmic portions show slight haematoxylin staining at the outer edges (arrow in B). Immunofluorescence staining of protruding portions shows a large amount of tubulin and OCT4 and SOX2 expression (arrows in C and D). Live cell releases a small non-nucleated call and shows 2 more protrusions (arrows in E). Images in the lower panel show time-lapse of a cellular protrusion (arrows) with extremely active motion. Scale bars in A-E=10 μm, lower panel=20 μm.

FIGS. 33A-33C show multiple NPRCPs are fused to form nucleus (33A-33B). DAPI stains on irregular shaped nucleus. The images indicate that DNA appears on multiple NPRCPs, which locate close to each other. DNA becomes a nucleus after rearrange or nuclear programming. Bar in 33B=5 μm. Large fused cellular structures differentiate into multiple cells. These cells are located in the large structure that express strongly to oct4 (red). No clear cytoplasmic components were observed. Bar in 33C=10 μm.

FIG. 34 shows evidence of nuclear programming in PFDNC-derived pre-cells. NPRCPs and their aggregated products, PFDNCs, do not have DNA or nucleus. They collect DNA from the nucleus of the eukaryotic cells or from circulation DNA fragments and than undergo nuclear programming. Images show the pre-cell with no DNA (left in upper panel), scattered DNA (left in lower panel) or circle-like form DNA (lower right), indicating that they are undergo nuclear programming to become eukaryotic cells. Scale bars=10 μm.

Evidence of the Presence of Other Types of Blood NPRCPs that Regenerate Other Blood Type Cells (FIG. 35).

Upper images show two H&E stain cellular structure that do not have aggregated particles, suggesting they can transform into other type of cells, but not mesenchymal-like cells. Scale bars=5 μm. Lower images show the electron microscopy of the NPRCPs that are direct transform into one type of blood cells. These particles are in the small round shape and the dense center become fading when they become larger. No nucleus is seen in these particles, indicating that they are not cells.

Evidence of the Techniques to Purify NPRCPs.

FIGS. 36A, 36B show the isolation of purified NPRCPs. NPRCPs are different from PFDNCs by their sizes and their motions. The purified NPRCPs (left) and the mixture of PFDNCs, differentiated PFDNC and pre-cells (right) are shown. These images indicate that we have the techniques to purify NPRCPs.

Table for microRNA Results. MicroRNA array were performed using 3 collections of NPRCPs (FIG. 36A) and 3 groups of middle sized particles or pre-cells (FIG. 36B). MicroRNA expression between these two groups was compared and analyzed. The data that has the p-value smaller than 0.05, was selected. The microRNA that highly expressed in the NPRCPs compared to the group 2 were listed in Table 1.

TABLE 1
Increased expression in NPRCPs (Fraction 1).

| Systematic Name | p values | Fold change (sm/mid) |
|---|---|---|
| hsa-miR-1182 | 0.001203935 | 353.1884607 |
| hsa-miR-1224-5p | 0.049823428 | 6.447147211 |
| hsa-miR-1225-5p | 0.048239009 | 3.74078511 |
| hsa-miR-1275 | 0.003773665 | 6.921440629 |
| hsa-miR-1299 | 0.02951881 | 188.9782154 |
| hsa-miR-187* | 0.026537525 | 214.4342184 |
| hsa-miR-1973 | 0.016582971 | 10.41720786 |
| hsa-miR-2861 | 0.001985627 | 6.659434362 |
| hsa-miR-30c-2* | 0.046507878 | 106.2203336 |
| hsa-miR-3138 | 0.048678475 | 3.88324654 |
| hsa-miR-3141 | 0.042221975 | 7.743632559 |
| hsa-miR-3154 | 0.002517634 | 347.9041826 |
| hsa-miR-320c | 0.01561311 | 7.627495031 |
| hsa-miR-3652 | 0.001469653 | 3.985633309 |
| hsa-miR-3656 | 0.032081715 | 5.846592219 |
| hsa-miR-3663-3p | 0.049590001 | 5.357489447 |
| hsa-miR-3667-5p | 0.020385388 | 6.183164943 |
| hsa-miR-3679-5p | 0.000951021 | 9.997583776 |
| hsa-miR-3682 | 0.025236989 | 182.1820054 |
| hsa-miR-3713 | 0.044000977 | 27.25974927 |
| hsa-miR-3917 | 0.009050322 | 143.6812606 |
| hsa-miR-422a | 0.001457302 | 560.7007826 |
| hsa-miR-4281 | 0.032762398 | 7.854478891 |
| hsa-miR-4298 | 0.040169129 | 11.23521903 |
| hsa-miR-4314 | 0.002734851 | 528.2650668 |
| hsa-miR-451 | 0.021309953 | 3.774193853 |
| hsa-miR-483-5p | 0.028384482 | 4.762996848 |
| hsa-miR-498 | 0.01338011 | 531.7321282 |
| hsa-miR-526b | 0.000879265 | 236.6497485 |
| hsa-miR-572 | 0.030155133 | 3.624191691 |
| hsa-miR-575 | 0.013532544 | 5.5937037 |
| hsa-miR-638 | 0.001917471 | 5.464083276 |
| hsa-miR-642b | 0.037094582 | 5.198708919 |
| hsa-miR-711 | 0.030542194 | 242.4120392 |
| hsa-miR-765 | 0.022681406 | 4.671387622 |
| hsa-miR-769-3p | 0.017784385 | 36.59980236 |
| hsa-miR-877 | 0.015163123 | 814.0336501 |
| hsa-miR-936 | 0.002865934 | 246.6946887 |

The microRNA that is significantly lower expressed in the NPRCPs compared to the mixed group 2 were listed in Table 2.

TABLE 2
Decreased microRNA in the NPRCPs.

| Systematic Name | p values | fold change |
|---|---|---|
| hsa-miR-101 | 0.022935032 | 0.369054487 |
| hsa-miR-10a | 0.001813109 | 0.002680511 |
| hsa-miR-125b | 0.022370144 | 0.013085089 |
| hsa-miR-1280 | 0.025678388 | 0.764302041 |
| hsa-miR-132 | 0.001819004 | 0.008360847 |
| hsa-miR-142-3p | 0.003623464 | 0.111540138 |
| hsa-miR-142-5p | 0.000550144 | 0.099842699 |
| hsa-miR-150 | 0.005823855 | 0.024283718 |
| hsa-miR-155 | 0.001534043 | 0.154964283 |
| hsa-miR-16-2* | 0.010518594 | 0.020292566 |
| hsa-miR-181a | 0.027282643 | 0.120399121 |
| hsa-miR-181a* | 5.41E−05 | 0.003181862 |
| hsa-miR-181c | 5.53E−05 | 0.002725663 |
| hsa-miR-181d | 0.000232978 | 0.007711455 |
| hsa-miR-186 | 0.02157393 | 0.300260503 |
| hsa-miR-196b | 0.025217353 | 0.004745985 |
| hsa-miR-20a* | 0.000291297 | 0.004766841 |
| hsa-miR-21* | 0.037805994 | 0.027584364 |
| hsa-miR-222 | 0.019902518 | 0.021088295 |
| hsa-miR-223* | 0.028289492 | 0.028760982 |
| hsa-miR-26a | 0.049679221 | 0.258899405 |
| hsa-miR-29b-1* | 0.043097858 | 0.025937203 |
| hsa-miR-29c | 0.026323809 | 0.24486609 |
| hsa-miR-30e | 0.021902972 | 0.354606425 |
| hsa-miR-32 | 0.000524589 | 0.002208287 |
| hsa-miR-338-3p | 0.038204157 | 0.204291358 |
| hsa-miR-33a | 0.006637905 | 0.00990206 |
| hsa-miR-342-3p | 0.046305169 | 0.132303076 |
| hsa-miR-342-5p | 0.012817558 | 0.002662788 |
| hsa-miR-361-3p | 0.012714334 | 0.005598873 |
| hsa-miR-362-3p | 0.017847115 | 0.010823865 |
| hsa-miR-362-5p | 0.009995274 | 0.009957682 |
| hsa-miR-363 | 0.034283243 | 0.248664279 |
| hsa-miR-424 | 0.015477556 | 0.159115971 |
| hsa-miR-4317 | 0.027239701 | 0.028161086 |
| hsa-miR-493* | 0.048114183 | 0.067318427 |
| hsa-miR-500a* | 0.036053213 | 0.061198304 |
| hsa-miR-505 | 0.029666878 | 0.034698634 |
| hsa-miR-532-3p | 0.003966572 | 0.011654216 |
| hsa-miR-551b | 0.014068549 | 0.011314071 |
| hsa-miR-590-5p | 0.008681092 | 0.279153862 |
| hsa-miR-99a | 5.57E−05 | 0.003795368 |

Evidence of Isolation of Mouse NPRCPs.

FIGS. 37A-37J to 38 show that mouse NPRCPs can be isolated by using similar methods as used for human NPRCPs. GFP-transgenic mice-isolated mouse NPRCPs express Oct4, sox2, DDX4, actin, tubulin and GFP, have sizes from about 1 to about 5 μm.

Evidence of Mouse Kidney Regeneration by Transplanted NPRCPs.

FIGS. 39A-39G up to 42A-42J show that NPRCPs migrate to the mouse ischemic kidney at day 1 after transplantation. Tiny GFP-expressing particles that are co-expressing Oct4, sox2, and DDX4, and extravasated materials are grouped together.

FIGS. 43A, 43B to 44F show that, 1 week after transplantation, GFP-positive NPRCPs fuse into large patch-like structures that form into renal duct structures. The fused patches also co-express sox2 and Oct4.

Evidence of NPRCPs Direct Differentiation into Kidney Interstitial Cells.

FIG. 45 shows interstitial tissue regeneration. Tiny cells co-expressed GFP (arrows) and DDX4 in the interstitial areas at 1 week after transplantation in mice with ischemia-damaged kidneys.

Evidence of NPRCPs Regenerating Kidney Glomeruli.

FIGS. 46A-46H show the glomeruli regeneration. Week 1 after transplantation in mice with ischemia-damaged kidneys. GFP and DDX4 are co-expressed as small particles in a non-nuclear area. Week 2 after transplantation. GFP-positive glomerulus co-expressed Oct4. DAPI staining shows no nuclei. H&E staining shows possible regenerative stages of glomeruli from day 1 to week 4. FIG. 47 shows that extravasated GFP-expressing (brown color) NPRCPs that arrange into a glomeruli structure without nuclei.

Evidence of NPRCPs Regenerate Kidney Ducts.

FIG. 48 shows renal tubule cells (black arrows) from day 1 to week 4 after NPRCP transplantation. The regenerated tubules show brush border structures positive for GFP (circles).

Evidence of NPRCPs Regenerate Neurons.

FIGS. 51-52 show appearances of GFP-expressing NPRCPs in the ischemic damages brains 4 days after transplantation. Both DAB staining and fluorescent staining methods confirm that GFP- and nestin-expressing NPRCPs appeared in the brain 4 days later.

FIG. 53 show that aggregated NPRCPs form into nestin-expressing new neurons. The nuclear materials in the aggregated GFP-expressing NPRCPs fuse, undergo nuclear programming, and become the nucleus of the new neuron. Two nuclei (arrows in DAPI) were enlarged (the lower images). It clear shows that these two nuclei are formed by small dots-shaped DAPI particles. In addition, GFP-expressing NPRCPs are express nestin, indicating they form into neuron stem cells.

FIG. 55 shows the appearance of dendrites 7 days after transplantation. The aggregated NPRCPs start to protrude the dendrites on the 7 days after the transplantation. Meanwhile, dots-shaped nestin have linked together and become fiber-like structures.

FIGS. 56-59 show the mechanism of dendrite formation from early to late stages. At early stage (FIG. 56), NPRCPs are scattered in certain areas and form into a round shape. Nestin is expressed as tiny dots and also scattered in the same area. Further (FIG. 57), NPRCPs protrude fine fibers that connect to each other to form dendrites. GFP-expressing NPRCPs that have lost the small vesicle shape and become either dot or line shapes. Oct4 is expressed in the core area of this GFP-expressing structure, suggesting that is going to be the newly formed nucleus. These data also indicate the newly formed cell is a stem cell. Later (FIG. 58-59), dendrites are formed before the nucleus is fully formed. Immunofluorescent and immunohistological images further confirm that the dendrites of the new neurons are formed by the connection of NPRCPs and further differentiation. A nucleus can be seen in the DAB stained image (arrow, lower right), which stains weak to haematoxylin compared to other nuclei.

Evidence of Axon Regeneration in Mouse Brain Ischemic Models.

FIGS. 60-65 show that, except forming dendrites, NPRCPs also aggregate to form axons. However, axon formation appears slower and also in different patterns. The aggregated NPRCPs are surrounded by cells, or inside the duct-like structures. GFP and nestin are co-expressed on an axon-like structure 10 weeks after NPRCP transplantation. This data indicate that NPRCPs can regenerate axon, however, in a slower procedure.

Evidence of Hair Bulb Regeneration by Transplanted Mouse NPRCPs.

FIGS. 66-67 and 69 show that NPRCPs form into hair bulbs. Hair bulbs are formed in the circulation and tissues before reach the epithelial layers. Images show 2-day wound stained to GFP Oct4 and H&E. NPRCPs were extravasated at the wound edge with the shape of hair bulbs.

Evidence of Mouse Epidermal Regeneration by Transplanted NPRCPs.

FIG. 68 show that NPRCPs regenerate epithelial cells. GFP, Oct4 and DDX4-expressing NPRCPs appear at the epithelial layer, not at the basal layer but also at the surface layer.

Evidence of Mouse Hair Follicle Regeneration.

FIG. 70 show that new hair follicles express GFP, DDX4 and oct4 in wound sections, evidencing that these hair follicles are derived from NPRCPs.

FIGS. 71-72 show that NPRCPs regenerate connective tissues. Aggregated NPRCPs locate in the damaged dermal area. These images indicate that NPRCPs regenerate dermis.

Evidence of Smooth Muscle Regeneration.

FIGS. 73-74 show that NPRCPs regenerate smooth muscle. Small non-nucleated cells that line up to form a larger structure. Newly formed muscle fibers express both oct4 and GFP, supporting that these muscle fibers are derived from NPRCPs.

Evidence of Cardiomyocyte Regeneration.

NPRCPs aggregate into groups after migrate into heart tissues. In 2-week heart sections (FIGS. 75-77), grouped NPRCPs fuse into cardioblasts that also express smooth muscle actin, the stem cell marker. Smooth muscle actin stains as particle-shape, not fiber-like, indicating the fiber-shape is expressed on the differentiated cells. DAPI stain indicates that the newly fused NPRCPs are forming nuclei. By 8 weeks after the transplantation (FIGS. 78-80), only GFP-expressing cardiac-myoblasts are found in the heart.

Evidence of NPRCPs Regenerating Intestinal Crypt Stem Cells.

It is well known that the intestinal epithelial cells at the tip of villa are migrated from the cell at the bottom, or crypts area. NPRCPs were transplanted via tail-vein injection. Small intestine was collected 7 days after NPRCP transplantation. Under lower magnification, aggregated GFP-expressing NPRCPs were found only at the crypt areas, indicating NPRCPs transform into crypt intestinal stem cells (FIGS. 81-85).

Evidence of Hepatocyte Regeneration by NPRCPs.

Grouped and scattered GFP-expressing NPRCPs were found in the liver after transplantation (FIGS. 86A-86D). Some of these grouped NPRCPs also express Oct4. Higher magnified image shows that GFP-expressing NPRCPs are grouped together or surround the nucleus. This data indicate that, except fusion, NPRCP have the function of induce the differentiated cells to undergo the nuclear programming and become stem cells. However, the mechanism of induced stem cells by NPRCPs is not by gene modification but may be via regulating the gene expression.

FIGS. 87-91 show pancreatic regeneration. Insulin-producing non-nucleated cells are derived from blood. NPRCPs were transplanted to the diabetic mice. 2 days after transplantation, GFP-expressing NPRCPs were found grouped in the pancreatic tissues. Insulin stained as fine particles in the GFP-expressing NPRCPs. A hollow structure can be seen in the center of aggregated NPRCPs, indicating that the nucleus of this cell has not formed yet. This data also evidence that insulin is expressed in the fully differentiated cells, while GFP is expressed before the cells are differentiated. There is a time gap between insulin and GFP expression. These data indicate that pancreatic B cells are not stem cells. These cells are fully differentiated and do not have the co-expression with the stem cell markers.

Evidence of NPRCPs or PFDNCs Induce the Nucleated Cells to Form into Stem Cells.

FIG. 92 shows long-tailed NPRCPs or PFDNCs that co-express sox2, Oct4 and DDX4 in in vivo. This morphology evidences that NPRCPs or PFDNCs release the transcription factors or other regulatory factor for reprogramming the nucleated cells. Because PFDNCs can easily get inside the nucleated cells, they may release these regulatory factors, such as oct4, sox2, DDX4 and other unidentified proteins. Released oct4, sox2 and DDX4 can induce toxic or stressed cells into healthy or stem cells by function on the downstream proteins to reprogramming these cells. In this case, the functions of NPRCP are by NPRCP's natural released RNA and proteins and not by gene transfection.

Although the invention has been illustrated and described with respect to one or more implementations, equivalent alterations and modifications will occur to others skilled in the art upon the reading and understanding of this specification and the annexed drawings. In addition, while a particular feature of the invention may have been disclosed with respect to only one of several implementations, such feature may be combined with one or more other features of the other implementations as may be desired and advantageous for any given or particular application.

The Abstract of the Disclosure is provided to allow the reader to quickly ascertain the nature of the technical disclosure. It is submitted with the understanding that it will not be used to interpret or limit the scope or meaning of the following claims.

What we claim is:

1-61. (canceled)

62. A composition of cultured and isolated non-platelet RNA-containing particles (NPRCPs) wherein the NPRCPs comprise at least one type of particle selected from short-rod shaped particles and long-tailed particles.

63. The cultured and isolated NPRCPs of claim 62, wherein the NPRCPs have a membrane that is not identical to that of a cell containing a cytoplasmic membrane and a nucleus.

64. The cultured and isolated NPRCPs of claim 62, wherein the NPRCPs comprise small RNAs or micro-RNA, or both small RNAs and micro-RNA.

65. The cultured and isolated NPRCPs of claim 62, wherein the NPRCPs comprise particles comprising one or more of the selected proteins: Oct4, DDX4, sox-2, CD29, CXCR4 and c-kit, CD45, and CD34.

66. The cultured and isolated NPRCPs of claim 62, wherein less than 5% NPRCPs comprise E-cadherin.

67. The cultured and isolated NPRCPs of claim 62, wherein NPRCPs comprise particles lacking DNA.

68. The cultured and isolated NPRCPs of claim 62, wherein NPRCPs comprise particles that lack a nucleus.

69. The cultured and isolated NPRCPs of claim 62, wherein NPRCPs further comprise at least 5 types of particles from 1 to 5 μm, wherein the least 5 types of particles are selected from (a) particles having a thin outer membrane, the thin outer membrane comprising a loose membrane or tight membrane; and (b) large core-like granulose type particles, loose membrane type particles, solid particle type particles, condensed material type particles, round and uniformed type particles, wherein the NPCRPs having a loose membrane are irregular shaped particles.

70. The cultured and isolated NPRCPs of claim 62, wherein fusion of two or more NPRCPs produce a particle comprising a loose outer membrane and lacking one or more of the selected components: a nucleus, a nuclear membrane, and a cytoplasmic membrane.

71. The cultured and isolated non-platelet RNA-containing particles (NPRCPs) of claim 62, wherein NPRCPs comprise particles isolated by centrifugation from about 200×g to about 6000×g.

72. The isolated non-platelet RNA-containing particles (NPRCPs) of claim 62, wherein said NPRCPs can be enriched in population or selectively enriched in expression level of a protein or enriched in both population and selectively enriched in expression level of a protein by in vitro culture with proper medium.

73. The isolated non-platelet RNA-containing particles (NPRCPs) of claim 62, wherein NPRCPs comprise particles characterized by being capable of producing downstream products.

74. A composition of cultured and isolated particle-fusion-derived non-nucleated cells (PFDNC), wherein the PFDNC is derived from one or more non-platelet RNA-containing particles (NPRCPs) as described in claim 62, comprises a loose outer membrane and lacks a nucleus or nuclei, a nuclear membrane and/or a cytoplasmic membrane;
wherein the PFDNC transforms into a eukaryotic cell by penetrating a eukaryotic cell to collect DNA for transformation;
wherein the PFDNC undergoes nuclear programming or reprogramming;
wherein the PFDNC comprise particles comprising small RNAs and micro-RNA and lack DNA; and
wherein the PFDNC expresses one or more markers comprising: Oct4, sox2 or tubulin.

75. A composition comprising cultured non-platelet RNA-containing particles (NPRCPs), selected from the following types of particles: irregular shaped particles with a loose membrane, round shaped particles with a tight membrane, short-rod shaped particles, and long-tailed particles.

76. The composition of claim 75, wherein NPRCPs comprise particles that contain small RNAs or micro-RNA, or both small RNAs and micro-RNA.

77. The composition of claim 75, wherein NPRCPs comprise particles selected from one of the following types of particles: granule type particles, loose membrane type particles, solid particles, short-rod particles, long-tailed particles, condensed type particles, round particles, and uniform particles.

78. The composition of claim 75, wherein NPRCPs comprise particles that comprise one or more of the selected proteins: Oct4, DDX4/VASA, sox-2, tubulin, CD29, CXCR4 and c-kit, CD45, CD34, and actin.

79. The composition of claim 75, wherein less than 5% NPRCPs comprise E-cadherin surface marker.

80. The composition of claim 75, wherein NPRCPs comprise particles that can be characterized by one or more of the selected properties: comprising do not contain DNAs; comprising lack a nucleus and nuclear membranes; comprising can be isolated from a biological sample by centrifugation from about 200×g to about 5000×g; comprising can be enriched in population or selectively enriched in expression level of a protein or enriched in both population and selectively enriched in expression level of a protein by in vitro culture with proper medium; comprising varying sizes categorized as small, middle and large and ranging from about 0.1 μm to about 10 μm; and comprising can be used to produce the downstream products of claim 1.

81. A composition comprising cultured particle-fusion-derived non-nucleated cell (PFDNC), wherein the PFDNC result from or are derived from one or more non-platelet RNA-containing particles (NPRCPs) wherein the particle-fusion-derived non-nucleated cell (PFDNC) comprises a loose outer membrane and lacks a nucleus or nuclei, a nuclear membrane and/or a cytoplasmic membrane.

82. The composition of claim 81, wherein the PFDNC is characterized by having one or more of the following properties: transforms a eukaryotic cell by penetrating the eukaryotic cell to collect DNA; undergoes nuclear programming or reprogramming; comprises small RNAs and micro-RNA and lack DNA; and wherein the PFDNC are isolated by about 200×g to about 5000×g centrifugation.

83. The composition of claim 81, wherein the PFDNC can be enriched in population or selectively enriched in expression level of a protein or enriched in both population and selectively enriched in expression level of a protein by in vitro culture with proper medium.

84. A method of regenerating cells or tissues in vivo comprising administering to a patient in need of regenerating cells or tissues an effective amount of non-platelet RNA-containing particles (NPRCPs), wherein the NPRCPs transform in an in vivo environment after transplantation or administration, thereby regenerating the cells or tissues.

85. The method of claim 84, wherein NPRCPs are selected from short-rod shaped particles and long-tailed particles.

86. The method of claim 84, wherein NPRCPs comprise particles that contain small RNAs and micro-RNA and lack DNA.

87. The method of claim 84, wherein NPRCPs further comprise particles that have large core-like granulose type, loose membrane type, solid particle type, condensed material type and round and uniformed types.

88. The method of claim 84, wherein NPRCPs comprise particles that contain one or more of the selected proteins: Oct4, DDX4/VASA, sox-2, CD29, CXCR4 and c-kit, CD45, and CD34.

89. The method of claim 84, wherein less than 5% NPRCPs comprises E-cadherin surface marker.

90. The method of claim 84, wherein the NPRCPs are selected from the following types of particles: particles lacking a nucleus; particles of varying sizes categorized as small, middle and large and ranged from about 0.1 μm to about 10 μm; and pluripotent particles that differentiate into multiple cell lineages, wherein the particles are obtained from a source selected from: autologous, heterologous, syngeneic, allogeneic and xenogeneic sources.

91. The method of claim 84, wherein NPRCPs are optionally cultured and expanded ex vivo prior to administering to a patient and are optionally cultured with desired differentiation and/or growth factors ex vivo, prior to administering to a patient.

92. The method of claim 84, wherein the tissue type is selected from one of the following tissue types: heart, kidney, brain, and skin.

93. The method of claim 92, wherein the regenerated cell or tissue type is selected from one or more of the following: kidney, renal ducts, renal glomeruli, renal tubule, brain, neuron, axon, skin, hair bulb, epithelial, hair follicle, dermis, muscle, smooth muscle, heart, cardioblast, cardiomyocyte, liver, hepatocyte, intestinal crypt, induced stem, pancreas, and insulin-producing non-nucleated.


SEQUENCE LISTING (80 sequences)

SEQ ID NO: 1 - RNA, 23 nt, Homo sapiens
gagggucuugggagggaugugac

SEQ ID NO: 2 - RNA, 19 nt, Homo sapiens
gugaggacucgggaggugg

SEQ ID NO: 3 - RNA, 22 nt, Homo sapiens
guggguacggcccagugggggg

SEQ ID NO: 4 - RNA, 17 nt, Homo sapiens
gugggggagaggcuguc

SEQ ID NO: 5 - RNA, 22 nt, Homo sapiens
uucuggaauucugugugaggga

SEQ ID NO: 6 - RNA, 22 nt, Homo sapiens
ggcuacaacacaggacccgggc

SEQ ID NO: 7 - RNA, 19 nt, Homo sapiens
accgugcaaagguagcaua

SEQ ID NO: 8 - RNA, 19 nt, Homo sapiens
ggggccuggcggugggcgg

SEQ ID NO: 9 - RNA, 22 nt, Homo sapiens
cugggagaaggcuguuuacucu

SEQ ID NO: 10 - RNA, 24 nt, Homo sapiens
uguggacagugagguagagggagu

SEQ ID NO: 11 - RNA, 19 nt, Homo sapiens
gagggcggguggaggagga

SEQ ID NO: 12 - RNA, 22 nt, Homo sapiens
cagaaggggaguugggagcaga

SEQ ID NO: 13 - RNA, 20 nt, Homo sapiens
aaaagcuggguugagagggu

SEQ ID NO: 14 - RNA, 18 nt, Homo sapiens
cggcuggaggugugagga

SEQ ID NO: 15 - RNA, 17 nt, Homo sapiens
ggcgggugcgggggugg

SEQ ID NO: 16 - RNA, 23 nt, Homo sapiens
ugagcaccacacaggccgggcgc

SEQ ID NO: 17 - RNA, 22 nt, Homo sapiens
aaagacccauugaggagaaggu

SEQ ID NO: 18 - RNA, 23 nt, Homo sapiens
ugaggauauggcagggaagggga

SEQ ID NO: 19 - RNA, 21 nt, Homo sapiens
ugaugauacagguggagguag

SEQ ID NO: 20 - RNA, 20 nt, Homo sapiens
gguauccguuuggggauggu

SEQ ID NO: 21 - RNA, 20 nt, Homo sapiens
gcucggacugagcagguggg

SEQ ID NO: 22 - RNA, 22 nt, Homo sapiens
acuggacuuagggucagaaggc

SEQ ID NO: 23 - RNA, 18 nt, Homo sapiens
gggucccggggagggggg

SEQ ID NO: 24 - RNA, 22 nt, Homo sapiens
cugggacaggaggaggaggcag

SEQ ID NO: 25 - RNA, 18 nt, Homo sapiens
cucugggaaaugggacag

SEQ ID NO: 26 - RNA, 22 nt, Homo sapiens
aaaccguuaccauuacugaguu

SEQ ID NO: 27 - RNA, 22 nt, Homo sapiens
aagacgggaggaaagaagggag

SEQ ID NO: 28 - RNA, 23 nt, Homo sapiens
uuucaagccagggggcguuuuuc

SEQ ID NO: 29 - RNA, 23 nt, Homo sapiens
cucuugagggaagcacuuucugu

SEQ ID NO: 30 - RNA, 20 nt, Homo sapiens
guccgcucggcgguggccca

SEQ ID NO: 31 - RNA, 19 nt, Homo sapiens
gagccaguuggacaggagc

SEQ ID NO: 32 - RNA, 25 nt, Homo sapiens
agggaucgcgggcggguggcggccu

SEQ ID NO: 33 - RNA, 22 nt, Homo sapiens
agacacauuuggagagggaccc

SEQ ID NO: 34 - RNA, 22 nt, Homo sapiens
gggacccagggagagacguaag

SEQ ID NO: 35 - RNA, 21 nt, Homo sapiens
uggaggagaaggaaggugaug

SEQ ID NO: 36 - RNA, 23 nt, Homo sapiens
cugggaucuccggggucuugguu

SEQ ID NO: 37 - RNA, 20 nt, Homo sapiens
guagaggagauggcgcaggg

SEQ ID NO: 38 - RNA, 22 nt, Homo sapiens
acaguagagggaggaaucgcag

SEQ ID NO: 39 - RNA, 21 nt, Homo sapiens
uacaguacugugauaacugaa

SEQ ID NO: 40 - RNA, 23 nt, Homo sapiens
uacccuguagauccgaauuugug

SEQ ID NO: 41 - RNA, 22 nt, Homo sapiens
ucccugagacccuaacuuguga

SEQ ID NO: 42 - RNA, 17 nt, Homo sapiens
ucccaccgcugccaccc

SEQ ID NO: 43 - RNA, 22 nt, Homo sapiens
ugugacugguugaccagagggg

SEQ ID NO: 44 - RNA, 23 nt, Homo sapiens
uguaguguuuccuacuuuaugga

SEQ ID NO: 45 - RNA, 21 nt, Homo sapiens
cauaaaguagaaagcacuacu

SEQ ID NO: 46 - RNA, 22 nt, Homo sapiens
ucucccaacccuuguaccagug

SEQ ID NO: 47 - RNA, 23 nt, Homo sapiens
uuaaugcuaaucgugauaggggu

SEQ ID NO: 48 - RNA, 22 nt, Homo sapiens
ccaauauuacugugcugcuuua

SEQ ID NO: 49 - RNA, 23 nt, Homo sapiens
aacauucaacgcugucggugagu

SEQ ID NO: 50 - RNA, 22 nt, Homo sapiens
accaucgaccguugauuguacc

SEQ ID NO: 51 - RNA, 22 nt, Homo sapiens
aacauucaaccugucggugagu

SEQ ID NO: 52 - RNA, 23 nt, Homo sapiens
aacauucauuguugucggugggu

SEQ ID NO: 53 - RNA, 22 nt, Homo sapiens
caaagaauucuccuuuugggcu

SEQ ID NO: 54 - RNA, 22 nt, Homo sapiens
uagguaguuuccuguuguuggg

SEQ ID NO: 55 - RNA, 22 nt, Homo sapiens
acugcauuaugagcacuuaaag

SEQ ID NO: 56 - RNA, 21 nt, Homo sapiens
caacaccagucgaugggcugu

SEQ ID NO: 57 - RNA, 21 nt, Homo sapiens
agcuacaucuggcuacugggu

SEQ ID NO: 58 - RNA, 22 nt, Homo sapiens
cguguauuugacaagcugaguu

SEQ ID NO: 59 - RNA, 22 nt, Homo sapiens
uucaaguaauccaggauaggcu

SEQ ID NO: 60 - RNA, 24 nt, Homo sapiens
gcugguuucauauggugguuuaga

SEQ ID NO: 61 - RNA, 22 nt, Homo sapiens
uagcaccauuugaaaucgguua

SEQ ID NO: 62 - RNA, 22 nt, Homo sapiens
uguaaacauccuugacuggaag

SEQ ID NO: 63 - RNA, 20 nt, Homo sapiens
aaaagcuggguugagagggu

SEQ ID NO: 64 - RNA, 22 nt, Homo sapiens
uccagcaucagugauuuuguug

SEQ ID NO: 65 - RNA, 21 nt, Homo sapiens
gugcauuguaguugcauugca

SEQ ID NO: 66 - RNA, 23 nt, Homo sapiens
ucucacacagaaaucgcacccgu

SEQ ID NO: 67 - RNA, 21 nt, Homo sapiens
aggggugcuaucugugauuga

SEQ ID NO: 68 - RNA, 23 nt, Homo sapiens
ucccccaggugugauucugauuu

SEQ ID NO: 69 - RNA, 22 nt, Homo sapiens
aacacaccuauucaaggauuca

SEQ ID NO: 70 - RNA, 24 nt, Homo sapiens
aauccuuggaaccuaggugugagu

SEQ ID NO: 71 - RNA, 22 nt, Homo sapiens
aauugcacgguauccaucugua

SEQ ID NO: 72 - RNA, 22 nt, Homo sapiens
cagcagcaauucauguuuugaa

SEQ ID NO: 73 - RNA, 17 nt, Homo sapiens
acauugccagggaguuu

SEQ ID NO: 74 - RNA, 22 nt, Homo sapiens
uuguacaugguaggcuuucauu

SEQ ID NO: 75 - RNA, 22 nt, Homo sapiens
augcaccugggcaaggauucug

SEQ ID NO: 76 - RNA, 22 nt, Homo sapiens
cgucaacacuugcugguuuccu

SEQ ID NO: 77 - RNA, 22 nt, Homo sapiens
ccucccacacccaaggcuugca

SEQ ID NO: 78 - RNA, 21 nt, Homo sapiens
gcgacccauacuugguuucag

SEQ ID NO: 79 - RNA, 22 nt, Homo sapiens
gagcuuauucauaaaagugcag

SEQ ID NO: 80 - RNA, 22 nt, Homo sapiens
aacccguagauccgaucuugug